(19) United States
(12) Patent Application Publication
(10) Pub. No.: US 2023/0360732 A1
(43) Pub. Date: Nov. 9, 2023

(54) SYSTEMS AND METHODS FOR ASSESSING AND IMPROVING THE QUALITY OF MULTIPLEX MOLECULAR ASSAYS

(71) Applicant: Nautilus Subsidiary, Inc., Seattle, WA (US)

(72) Inventors: Jarrett D. EGERTSON, Rancho Palos Verdes, CA (US); James SHERMAN, Kingston, WA (US); Vadim LOBANOV, Seattle, WA (US); Parag MALLICK, San Mateo, CA (US); James Henry JOLY, San Mateo, CA (US)

(21) Appl. No.: 18/301,891

(22) Filed: Apr. 17, 2023

Related U.S. Application Data
(60) Provisional application No. 63/334,586, filed on Apr. 25, 2022.
(60) Provisional application No. 63/385,722, filed on Dec. 1, 2022.

(51) Int. Cl. G16B 30/10; G16B 40/00; G16B 50/30

(52) U.S. Cl. CPC G16B 30/10; G16B 40/00; G16B 50/30

(57) ABSTRACT

A method of identifying extant proteins, including (a) inputting to a computer processor: (i) a plurality of empirical binding profiles, individual empirical binding profiles including empirical binding outcomes for binding of an extant protein to a plurality of different affinity reagents, (ii) a plurality of candidate outcome profiles, individual candidate outcome profiles including binding outcomes for binding of a candidate protein to the plurality of different affinity reagents, and (iii) a plurality of pseudo outcome profiles, individual pseudo outcome profiles including a rearrangement of a candidate outcome profile; (b) performing a process in the computer processor to identify extant proteins based on the empirical binding profiles of the extant proteins and the plurality of candidate outcome profiles; and (c) performing a process in the computer processor to determine a false discovery statistic for the extant proteins based on the plurality of pseudo outcome profiles.

### CROSS-REFERENCE TO RELATED APPLICATIONS

This application claims the benefit of U.S. Provisional Application No. 63/334,586 filed on Apr. 25, 2022, and U.S. Provisional Application No. 63/385,722 filed on Dec. 1, 2022, each of which applications is incorporated by reference in its entirety. Any and all applications for which a foreign or domestic priority claim is identified in the Application Data Sheet as filed with the present application are hereby incorporated by reference under 37 CFR 1.57.

### SEQUENCE LISTING

The instant application contains a Sequence Listing which has been submitted electronically in XML format and is hereby incorporated by reference in its entirety. Said XML copy, created on Apr. 17, 2023, is named NBIOT.012A.xml and is 1.9 kilobytes in size.

### BACKGROUND

The proteome is among the most dynamic and valuable sources of biological insight. Current proteomics techniques are limited in their sensitivity and throughput, covering at best 35% of the human proteome in a single experiment (see Blume et al., Nat Commun 11, 3662 (2020) and Clark et al., Cell 180, 207 (2020), each of which is incorporated herein by reference). Despite the wealth of insights gained from now routine genomics and transcriptomics studies in biomedical research, a large gap remains between genome/transcriptome and phenotype. Proteomics is crucial to bridging this gap as proteins constitute the main structural and functional components of cells. However, protein sequencing technologies lag behind DNA sequencing technologies, in part due to the complex nature of proteins and proteomes as well as the high dynamic range (˜10⁹) in the quantities of different proteins present at any given time in any given cell (see Aebersold et al., Nat Chem Biol 14, 206-214 (2018), which is incorporated herein by reference). Moreover, about 10% of the proteins predicted to comprise the human proteome have not been confidently observed at all (see Omenn et al., J Proteome Res 19, 4735-4746 (2020) and Adhikari et al., Nat Commun 11, 5301 (2020), each of which is incorporated herein by reference).

Recently, single-molecule identification has been postulated as a method to analyze small samples (including single cells) and rare proteins (see Alfaro et al., Nat Methods 18, 604-617 (2021) and Restrepo-Perez et al., Nat Nanotechnol 13, 786-796 (2018), each of which is incorporated herein by reference). Traditional bulk identification techniques like mass spectrometry and immunoassays have been adapted towards detection of single proteins (see Keifer & Jarrold, Mass Spectrom Rev 36, 715-733 (2017) and Risin et al., Nat Biotechnol 28, 595-599 (2010), each of which is incorporated herein by reference). Several concepts have been proposed to achieve single-molecule protein sequencing. These all use sequential processes to determine the positional information of amino acids within proteins e.g., Edman-type degradation (Swaminathan, et al. Nat Biotechnol (2018) and Swaminathan, et al., PLoS Comput Biol 11, e1004080 (2015), each of which is incorporated herein by reference) or directional protein translocation through a nanopore channel (Kolmogorov, et al., PLoS Comput Biol 13, e1005356 (2017), each of which is incorporated herein by reference).

### SUMMARY

The present disclosure provides a method of identifying extant proteins. The method can include steps of: (a) providing inputs to a computer processor, the inputs including (i) a plurality of empirical outcome profiles, individual empirical outcome profiles of the plurality of empirical outcome profiles each including a plurality of empirical measurement outcomes for an extant protein, individual empirical measurement outcomes of the plurality of empirical measurement outcomes each including a measured outcome for reaction of the extant protein with a different assay reagent, (ii) a plurality of candidate outcome profiles, individual candidate outcome profiles of the plurality of candidate outcome profiles each including a plurality of statistical measures for a candidate protein, wherein the candidate proteins are known or suspected of being present in the sample, and (iii) a plurality of pseudo outcome profiles, individual pseudo outcome profiles of the plurality of pseudo outcome profiles each including a plurality of statistical measures that is known to not occur for any of the candidate proteins; (b) performing a process in the computer processor to identify extant proteins of the plurality of different extant proteins based on the empirical outcome profiles of the extant proteins and the plurality of candidate outcome profiles; and (c) performing a process in the computer processor to determine a false discovery statistic for the extant proteins based on the plurality of empirical outcome profiles and the plurality of pseudo outcome profiles. Optionally, the empirical outcome profiles are empirical binding profiles and individual empirical measurement outcomes of the empirical outcome profiles include a measure for binding of an extant protein to a given affinity reagent. As a further option, individual candidate outcome profiles of the plurality of candidate outcome profiles each include a plurality of binding statistics for a candidate protein, individual binding statistics of the plurality of binding statistics comprising a measure of uncertainty or variation for binding of the candidate protein with a given affinity reagent.

Another method of identifying extant proteins can include the steps of: (a) contacting an array of different extant proteins with a plurality of different affinity reagents, wherein individual addresses of the array are each attached to an extant protein; (b) acquiring empirical binding profiles from the individual addresses, the empirical binding profiles each including a plurality of binding outcomes for binding of an extant protein at one of the individual addresses to the plurality of different affinity reagents; (c) providing a plurality of candidate outcome profiles, individual candidate outcome profiles of the plurality of candidate outcome profiles each including a plurality of statistical measures for a candidate protein, wherein the candidate proteins are known or suspected of being present in the sample; (d) providing a plurality of pseudo outcome profiles, individual pseudo outcome profiles of the plurality of pseudo outcome profiles each including a plurality of statistical measures that is known to not occur for any of the candidate proteins; (e) identifying extant proteins of the array based on the empirical binding profiles of the extant proteins and the plurality of candidate outcome profiles; and (f) determining a false discovery statistic for the extant proteins based on the empirical binding profiles of the extant proteins and the plurality of pseudo outcome profiles.

The present disclosure further provides a system for identifying proteins, including (a) a detector configured to acquire signals from a plurality of binding reactions occurring between a plurality of different affinity reagents and a plurality of extant proteins in a sample; (b) a database including: (i) a plurality of candidate outcome profiles, the individual candidate outcome profiles of the plurality of candidate outcome profiles each including a plurality of representations of binding outcomes for binding of a candidate protein to the plurality of different affinity reagents, and (ii) a plurality of pseudo outcome profiles, individual pseudo outcome profiles of the plurality of pseudo outcome profiles each including a plurality of statistical measures that is known to not occur for any of the candidate proteins; (c) a computer processor configured to: (i) communicate with the database, (ii) acquire a plurality of empirical binding profiles from the signals, wherein each of the empirical binding profiles includes a plurality of binding outcomes for binding of an extant protein of the sample to the plurality of different affinity reagents; (iii) identify extant proteins of the plurality of different extant proteins based on the empirical binding profiles of the extant proteins and the plurality of candidate outcome profiles; and (iv) compute a false discovery statistic for the extant proteins based on the empirical binding profiles of the extant proteins and the plurality of pseudo outcome profiles.

The present disclosure provides a protein assay method, including steps of: (a) inputting to a computer processor measurement outcomes for reactions of a plurality of assay reagents to an extant protein; (b) inputting to the computer processor a database including a plurality of candidate proteins; and (c) in the computer processor: (i) adding a measurement outcome of step (a) to an outcome profile of the extant protein; (ii) determining a collection of probabilities for each of the candidate proteins in the database producing the outcome profile; (iii) determining information entropy for the collection of probabilities; and (iv) repeating steps (i) through (iii).

A protein assay method can include steps of: (a) inputting to a computer processor binding outcomes for binding of a plurality of affinity reagents to an extant protein; (b) inputting to the computer processor a database including a plurality of candidate proteins; (c) inputting to the computer processor a binding model for each of the different affinity reagents; and (d) in the computer processor: (i) adding a binding outcome of step (a) to a binding profile of the extant protein; (ii) evaluating the binding model to determine a collection of probabilities for each of the candidate proteins in the database producing the binding profile; (iii) determining information entropy for the collection of probabilities; and (iv) repeating steps (i) through (iii).

The present disclosure provides a method for conducting a protein assay, including the steps of: (a) contacting an array of different extant proteins with assay reagents, wherein individual addresses of the array are each attached to a single extant protein of the different extant proteins; (b) determining a measurement outcome for reaction of the assay reagents at each of the individual addresses of the array; (c) providing a database including a plurality of candidate proteins; and (d) for an individual address in the array: (i) adding a measurement outcome of step (b) to an outcome profile of the individual address; (ii) determining a collection of probabilities for each of the candidate proteins in the database producing the outcome profile; (iii) determining information entropy for the collection of probabilities; and (iv) repeating steps (i) through (iii).

Yet another method for conducting a protein assay can include steps of (a) contacting an array of different extant proteins with a plurality of different affinity reagents, wherein individual addresses of the array are each attached to a single extant protein of the different extant proteins, and wherein the different affinity reagents recognize different extant proteins in the array; (b) determining a binding outcome for each of the different affinity reagents at each of the individual addresses of the array; (c) providing a database including a plurality of candidate proteins; (d) providing a binding model for each of the different affinity reagents; and (e) for an individual address in the array: (i) adding a binding outcome of step (b) to a binding profile of the individual address; (ii) evaluating the binding model to determine a collection of probabilities for each of the candidate proteins in the database producing the binding profile; (iii) determining information entropy for the collection of probabilities; and (iv) repeating steps (i) through (iii).

Provided herein is a detection system including: (a) a detector configured to detect measurement outcomes for reactions of a plurality of assay reagents with an array of addresses, each of the addresses having an extant protein of a plurality of different extant proteins; (b) a database including a plurality of candidate proteins; and (c) a computer processor configured to: (i) add a measurement outcome of (a) to an outcome profile of an individual address of the array; (ii) determine a collection of probabilities for each of the candidate proteins in the database producing the outcome profile; (iii) determine information entropy for the collection of probabilities; and (iv) repeat (i) through (iii). Optionally, the computer processor is configured to output an identity for the extant protein at the individual address. Alternatively or additionally, the computer processor can be configured to output a measure of the information entropy.

In some configurations, a detection system can include: (a) a detector configured to detect binding outcomes for binding of a plurality of affinity reagents to an array of addresses, each of the addresses having an extant protein of a plurality of different extant proteins; (b) a database including a plurality of candidate proteins; (c) a binding model for each of the different affinity reagents; and (d) a computer processor configured to: (i) add a binding outcome of (a) to a binding profile of an individual address of the array; (ii) evaluate the binding model to determine a collection of probabilities for each of the candidate proteins in the database producing the binding profile; (iii) determine information entropy for the collection of probabilities; and (iv) repeat (i) through (iii). Optionally, the computer processor is configured to output an identity for the extant protein at the individual address, wherein the extant protein is identified as a candidate protein in the database. Alternatively or additionally, the computer processor can be configured to output a measure of the information entropy.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF THE DRAWINGS

FIG. 1A shows a workflow from sample preparation to data analysis for a method of identifying proteins.

FIG. 1B shows a depiction of protein decoding resulting in identification of the protein at location A1 as EGFR.

FIG. 1C shows repeated sequential affinity reagent measurements on EGFR showing five unique binding patterns and one off-target binding event.

FIG. 1D shows number of affinity reagents sufficient for 90% human proteome coverage with variation in length of epitope (dimer, trimer, tetramer) and number of epitopes bound by each multi-affinity reagent (asterisk indicates a value >2,000).

FIG. 1E shows proteome coverage achieved as affinity reagent cycles are measured using either affinity reagents targeting trimer epitopes optimized for the human proteome or one of 20 random sets of trimer targets.

FIG. 1F shows proteome coverage for human, mouse, yeast, and E. coli proteomes measured with an affinity reagent set optimized for human proteome coverage.

FIG. 2A shows coverage of the human proteome for affinity reagents of varying binding affinity.

FIG. 2B shows coverage of the human proteome for affinity reagents of varying binding affinity with non-specific binding to an array surface. Circle area is proportional to proteome coverage (also labeled on circle).

FIG. 2C shows impact of mischaracterization of affinity reagent binding on proteome coverage for varying fraction of unknown high-affinity epitope targets. All error bars are standard deviation across five replicates.

FIG. 2D shows impact of mischaracterization of affinity reagent binding on proteome coverage for varying fraction of false high-affinity epitope targets identified. All error bars are standard deviation across five replicates.

FIG. 2E shows impact of mischaracterization of affinity reagent binding on proteome coverage for systematic measurement error in binding probability. All error bars are standard deviation across five replicates.

FIG. 2F shows impact of mischaracterization of affinity reagent binding on proteome coverage for random measurement error in binding probability. All error bars are standard deviation across five replicates.

FIG. 3A shows dynamic range of protein quantification for blood plasma with varying protein array size. Data are plotted in order of decreasing protein abundance from top to bottom. Dynamic range is the protein abundance divided by the most abundant protein in sample. The outer width of the contours indicates the percentage of proteins at that abundance deposited on the protein array (one or more copies). The inner width of the contours indicates the percentage of proteins at that abundance detected by the decoding method. Percentages are computed over a rolling window of 51 proteins. Horizontal gray bars indicate 100%.

FIG. 3B shows dynamic range of protein quantification for HeLa cells with varying protein array size. Data are presented as set forth above for FIG. 3A.

FIG. 3C shows reproducibility of quantification (coefficient of variation computed across five replicates) compared to protein abundance for plasma as contour plots (density iso-proportional contours) with marginal histograms.

FIG. 3D shows reproducibility of quantification (coefficient of variation computed across five replicates) compared to protein abundance for HeLa cells as contour plots (density iso-proportional contours) with marginal histograms.

FIG. 3E shows concordance of quantity of proteins (number of copies identified) measured by the decoding method with true count of protein on array for a single experimental replicate of plasma.

FIG. 3F shows concordance of quantity of proteins (number of copies identified) measured by the decoding method with true count of protein on array for a single experimental replicate of HeLa cells.

FIG. 4A shows impact of mischaracterization of affinity reagent binding on proteome coverage for varying fraction of unknown high-affinity (primary) epitope targets and low-to-medium affinity (secondary) epitope targets. All coverage measurements are the average over 5 replicates.

FIG. 4B shows varying fraction of false high-affinity (primary) and low-to-medium affinity (secondary) epitope targets identified. All coverage measurements are the average over 5 replicates.

FIG. 4C shows systematic measurement error in binding probability with varying fraction of the 300 total affinity reagents impacted by the corruption. All coverage measurements are the average over 5 replicates.

FIG. 4D shows random measurement error in binding probability with varying fraction of the 300 total affinity reagents impacted by the corruption. All coverage measurements are the average over 5 replicates.

FIG. 5A shows distribution of protein abundance among proteins in a sample, deposited on a protein array, and quantified by the decoding method in plasma measured on an array having 10¹⁰ protein-occupied addresses. Histogram counts for each group are averaged over five simulated replicate experiments. The displayed non-specific quant rate is the maximum percentage of proteins observed in any replicate with poor quantification (>10% signal arising from false identifications). The percent of proteins in the sample quantified is shown as a gray line. Mean proteome coverage is the percent of proteomes present in a sample detected by the decoding method (averaged across the five replicates). Error bars indicate standard deviation.

FIG. 5B shows distribution of protein abundance among proteins in a sample, deposited on a protein array, and quantified by the decoding method in depleted plasma measured on an array having 10¹⁰ protein-occupied addresses. Data was processed and presented for FIG. 5A.

FIG. 5C shows distribution of protein abundance among proteins in a sample, deposited on a protein array, and quantified by the decoding method in a HeLa cell line measured on an array having 10¹⁰ protein-occupied addresses. Data was processed and presented for FIG. 5A.

FIG. 5D shows distribution of protein abundance among proteins in a sample, deposited on a protein array, and quantified by the decoding method in plasma measured on an array having 10⁸ protein-occupied addresses. Data was processed and presented for FIG. 5A.

FIG. 5E shows distribution of protein abundance among proteins in a sample, deposited on a protein array, and quantified by the decoding method in depleted plasma measured on an array having 10⁸ protein-occupied addresses. Data was processed and presented for FIG. 5A.

FIG. 5F shows distribution of protein abundance among proteins in a sample, deposited on a protein array, and quantified by the decoding method in a HeLa cell line measured on an array having 10⁸ protein-occupied addresses. Data was processed and presented for FIG. 5A.

FIG. 6A shows sensitivity and specificity of the decoding method for non-depleted plasma. The probability threshold for protein identification was varied: log(threshold)=0, −1e-20, −1e-16, −1e-14, −1e-12, −1e-11, −1e-10, −1e-9, −1e-8, −1e-7, −1e-6, −1e-5, −1e-4, −1e-3, −1e-2, −0.1, −0.2, and −0.3. A low threshold resulted in higher sensitivity (proteins quantified) but also a higher rate of non-specific quantification (signals where 10% or more of identifications are false). A point is plotted indicating these metrics for each threshold assessed for each of 5 replicate samples (shown as varying shapes). Simulations were performed with datasets comprising 10¹⁰ protein-occupied addresses and 10⁸ protein-occupied addresses.

FIG. 6B shows sensitivity and specificity of the decoding method for depleted plasma. Data was processed and presented for FIG. 6A.

FIG. 6C shows sensitivity and specificity of the decoding method for a HeLa cell line. Data was processed and presented for FIG. 6A.

FIG. 7A shows dynamic range in abundance of proteins deposited on arrays of varying size for non-depleted plasma. Data are plotted in order of decreasing protein abundance from top to bottom. Dynamic range is the ratio of protein abundance to that of the most abundant in sample. Outer width of contours indicates percentage of proteins at that abundance deposited on array (1 or more copy) with the bar at the top of each contour corresponding to 100%. Percentages are computed over a rolling window of 51 proteins.

FIG. 7B shows dynamic range in abundance of proteins deposited on arrays of varying size for depleted plasma. Data was processed and presented for FIG. 7A.

FIG. 7C shows dynamic range in abundance of proteins deposited on arrays of varying size for HeLa cells. Data was processed and presented for FIG. 7A.

FIG. 8A shows dynamic range of protein quantification for a depleted blood sample evaluated using the decoding method. Protein abundance data are plotted in order of decreasing abundance from top to bottom. Dynamic range is the ratio of protein abundance to that of most abundant in sample. The outer width of the contours indicates the percentage of proteins at that abundance deposited on the array (one or more copies). The inner width of the contours indicates the percentage of proteins at that abundance detected by the decoding method. Percentages are computed over a rolling window of 51 proteins. Horizontal bars indicate 100%.

FIG. 8B shows reproducibility of quantification (CV % among five replicates) compared to protein abundance using a contour plot (density iso-proportional contours) with marginal histograms for a depleted blood sample evaluated using the decoding method.

FIG. 8C shows concordance of quantity of proteins (number of copies detected) with true count of protein on array for a single replicate of a depleted blood sample evaluated using the decoding method.

FIG. 8D shows distribution of fold-change error, which is the count of protein copies detected by the decoding method divided by copies of the depleted plasma proteins deposited on the array. Copies detected and copies deposited are averaged across five replicates measured.

FIG. 9A shows reproducibility and accuracy of quantification demonstrated for non-depleted plasma samples assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9B shows the concordance of quantity of proteins (number of copies identified) measured by the decoding method with true count of protein on array shown for a single replicate of non-depleted plasma. Copies detected and copies deposited are averaged across five replicates measured.

FIG. 9C shows the distribution of fold-change error, which is the count of protein copies identified by the decoding method divided by copies of the protein deposited on the array for the non-depleted plasma. Copies detected and copies deposited are averaged across five replicates measured.

FIG. 9D shows reproducibility and accuracy of quantification demonstrated for depleted plasma assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9E shows the concordance of quantity of proteins (number of copies identified) measured by the decoding method with true count of protein on array shown for a single replicate of depleted plasma. Copies detected and copies deposited are averaged across five replicates measured.

FIG. 9F shows the distribution of fold-change error, which is the count of protein copies identified by the decoding method divided by copies of the protein deposited on the array for the depleted plasma. Copies detected and copies deposited are averaged across five replicates measured.

FIG. 9G shows reproducibility and accuracy of quantification demonstrated for HeLa cells assayed in five replicates on arrays with 10⁸ protein-occupied addresses. The reproducibility of quantification (CV % among five replicates) is compared to protein abundance using a contour plot (density iso-proportional contours) with marginal histograms. Copies detected and copies deposited are averaged across five replicates measured.

FIG. 9H shows the concordance of quantity of proteins (number of copies identified) measured by the decoding method with true count of protein on array shown for a single replicate of HeLa cells. Copies detected and copies deposited are averaged across five replicates measured.

FIG. 9I shows the distribution of fold-change error, which is the count of protein copies identified by the decoding method divided by copies of the protein deposited on the array for HeLa cells. Copies detected and copies deposited are averaged across five replicates measured.

FIG. 10A shows the reproducibility of protein deposition and protein quantification across five replicates for non-depleted plasma measured on arrays with 10¹⁰ protein-occupied addresses. Protein quantity deposited is the total count of a protein that was successfully deposited on the array. Protein quantity measured is the number of times the protein was identified by the decoding method. The CV (%) of each of these quantities across the five replicates is computed for each unique protein detected in the sample and plotted using a contour plot to demonstrate the concordance of variation in protein counts deposited with variation in protein counts measured.

FIG. 10B shows the reproducibility of protein deposition and protein quantification across five replicates for HeLa cells measured on arrays with 10¹⁰ protein-occupied addresses. Data was processed and presented as set forth for FIG. 10A.

FIG. 11 shows fold-change measurement error distribution for proteins detected in plasma samples measured on 10¹⁰ protein-occupied addresses. Fold change error is the count of protein copies detected by the decoding method divided by copies of protein deposited on the array. Copies detected and copies deposited are averaged across five replicates measured.

FIG. 12 shows a computer system that is programmed or otherwise configured to implement a method set forth herein.

FIG. 13 shows a plot of the median entropy across all decoded proteins for simulated data decoding HeLa lysate with affinity reagents having a 50% epitope binding rate.

FIG. 14 shows a plot of decode trajectories for 25 individual addresses each resulting in an identification of 40S ribosomal protein S13 for simulated data decoding HeLa lysate with affinity reagents having a 50% epitope binding rate.

FIG. 15 shows a diagrammatic representation of a method for estimating false identification rate using pseudo binding profiles.

FIG. 16 shows a plot of estimated false identification rate vs. ground truth false identification rate for simulated data decoding HeLa lysate with affinity reagents having a 50% epitope binding rate.

FIG. 17 shows images acquired from an array of structured nucleic acid particles (SNAPS). The left panel shows images obtained from a region of the array using a fluorescent channel tuned to detect fluorescent labels on the SNAPs; the middle panel shows images obtained from the same region using a fluorescent channel tuned to detect fluorescently labeled affinity reagents that recognize a model protein attached to the SNAPs; and the right panel shows a composite of the images shown in the left and middle panels.

FIG. 18 plots the percent of SNAPs remaining relative to the amount present at the tenth cycle for 70 cycles of a protein detection assay.

FIG. 19 shows the sequence for model protein 1 (SEQ ID NO:1), predicted binding probabilities and observed binding rates of affinity reagents to model protein 1.

FIG. 20 shows individual binding events for ten individual addresses on an array that were identified as presenting model protein 1.

FIG. 21 shows a bar graph of normalized detection counts obtained from decoding results of a protein binding assay using a database of the 5 proteins listed on the x axis.

FIG. 22 shows a plot of the log 10 likelihood ratio for addresses in an array being identified as model protein 1 for each decoded cycle in a protein binding assay. Also shown are tabulated results.

FIG. 23 shows a plot of normalized significant detection rate vs. nearest protein similarity.

FIG. 24 shows a plot of nearest protein similarity in the human proteome vs. number of affinity reagents (“multi-affinity probes”).

### DETAILED DESCRIPTION

A protein can be detected using one or more affinity reagents having known or measurable binding affinity for the protein. For example, an affinity reagent can bind a protein to form a complex and a signal produced by the complex can be detected. A protein that is detected by binding to a known affinity reagent can be identified based on the known or predicted binding characteristics of the affinity reagent. For example, an affinity reagent that is known to selectively bind a candidate protein suspected of being in a sample, without substantially binding to other proteins in the sample, can be used to identify the candidate protein in the sample merely by observing the binding event. This one-to-one correlation of affinity reagent to candidate protein can be used for identification of one or more proteins. However, as the protein complexity (i.e. the number and variety of different proteins) in a sample increases, the time and resources to produce a commensurate variety of affinity reagents having one-to-one specificity for the proteins approaches limits of practicality.

The present disclosure provides methods, systems and compositions that can be employed to overcome these constraints. In particular configurations, the number of different proteins identified can exceed the number of affinity reagents used. For example, the number of proteins identified can be at least 5×, 10×, 25×, 50×, 100× or more than the number of affinity reagents used. As set forth in further detail herein, one or more extant proteins can be identified by (1) performing binding reactions using promiscuous affinity reagents that bind to multiple different candidate proteins suspected of being present in a given sample, (2) subjecting one or more extant proteins to a set of the promiscuous affinity reagents that, taken as a whole, produce an empirical binding profile for each extant protein, and (3) performing a decoding method that evaluates the empirical binding profile according to a binding model for binding of the promiscuous affinity reagents to a plurality of candidate proteins, thereby identifying each of the one or more of the extant proteins based on compatibility with a respective candidate protein.

Particular configurations of the methods set forth herein can employ promiscuous affinity reagents. Promiscuity of an affinity reagent is a characteristic that can be understood relative to a given population of proteins. Promiscuity can arise due to the affinity reagent recognizing an epitope that is present in a plurality of different proteins that are known or suspected of being in a sample, such as a human proteome sample. For example, a promiscuous affinity reagent may recognize epitopes having relatively short amino acid lengths such as dimers, trimers, tetramers, pentamers or hexamers, wherein the epitopes are expected to occur in a substantial number of different proteins in a proteome of a human or other species. Alternatively or additionally, a promiscuous affinity reagent can recognize different epitopes (i.e. epitopes having a variety of different structures), the different epitopes being present in a plurality of different proteins in a proteome sample. For example, a promiscuous affinity reagent can have a high probability of binding to a primary epitope target and lesser probability for binding to one or more secondary epitope targets, the secondary epitope targets having a different sequence of amino acids when compared to the primary epitope target. Optionally, the secondary epitope targets can be biosimilar to the primary epitope target, for example, in accordance with a BLOSUM62 scoring matrix.

Although performing a single binding reaction between a promiscuous affinity reagent and a complex protein sample, such as a human proteome sample, may yield ambiguous results regarding the identity of the different proteins to which it binds, the ambiguity can be resolved when the results are evaluated in a decoding method set forth herein. A plurality of binding outcomes obtained from measuring binding of a plurality of affinity reagents with one or more extant proteins can be input into a decoding method of the present disclosure to identify the most likely identity of that protein among a set of candidate proteins. The plurality of binding outcomes can be input into a decoding method along with information characterizing or identifying a plurality of candidate proteins (e.g. amino acid sequences of candidate proteins), and a binding model. The probability of each affinity reagent binding to every possible candidate protein can be evaluated using the binding model and the decoding method can output the identity of individual extant proteins. For example, the decoding process can output the most likely identity for an individual extant protein as the candidate protein that is most compatible with the observed binding outcomes for the extant protein according to the binding model.

A binding model of the present disclosure can be configured on an assumption that the characteristics for affinity reagents binding to extant proteins in a sample, even if unknown, can be treated as quantifiable random variables, and that uncertainty about the binding characteristics can be described by probability distributions. Parameters for a plurality of affinity reagents can be determined, for example, based on a priori knowledge about the affinity reagents (e.g. expected binding affinity for particular epitopes) and/or based on preliminary reactions performed using the affinity reagents (e.g. measurement of binding between the affinity reagents and one or more epitopes). The parameters of the affinity reagents can be treated as ‘priors’ that are input into a decoding process of the present disclosure. The parameters of the affinity reagents when combined with empirically determined binding outcomes and evaluated using a decoding method of the present disclosure can output a ‘posterior,’ the calculation of which involves computation of a distribution of likelihoods for the identity of each extant protein used for the empirical determination. The posteriors that are output by the decoding method can be used to update the priors that will be used as inputs to subsequent evaluations using the decoding method. Accordingly, the influence of unknowns and artifacts in early evaluation of affinity reagents can be diminished as further empirical measurements are made and the results evaluated by the decoding method. This updating cycle can provide the benefit of facilitating iterative improvement to the decoding method, thereby improving the accuracy of identifying or characterizing extant proteins.

The decoding method set forth herein may take into account characteristics of binding reactions that may otherwise adversely affect the accuracy with which proteins can be identified. For example, binding reactions carried out at single-molecule scale (e.g. detecting binding of affinity reagents to proteins that are individually resolved on a protein array) produce stochastic results. Moreover, non-specific binding of affinity reagents, for example, to the surface of an array to which proteins under observation are attached, can also produce errant results. Another example is bias or skew that can arise due to different lengths of proteins that are analyzed in a decoding method set forth herein. As set forth in further detail herein, a decoding method can be configured to account for stochasticity, non-specific binding, differences in protein length, or other factors for improved accuracy when identifying or characterizing proteins. For example, stochasticity can be accounted for by estimating protein likelihood using the decoding method. Similarly, differences in protein length can be accounted for by computing a normalization factor that deends jointly on candidate protein length and number of observed positive binding outcomes.

For ease of explanation, the compositions, systems and methods of the present disclosure are often exemplified herein in the context of characterizing proteins using binding measurements. The examples set forth herein can be readily extended to characterizing other analytes (e.g. as an alternative or addition to proteins), or to the performance of other reactions (e.g. as an alternative or addition to binding reactions).

The present disclosure provides compositions, systems and methods that can be useful in various configurations for characterizing analytes, such as proteins, nucleic acids, cells or moieties thereof, by obtaining multiple separate and non-identical measurements of the analytes. In particular configurations, the individual measurements may not, by themselves, be sufficiently accurate or specific to make the characterization, but an aggregation of the multiple non-identical measurements can allow the characterization to be made with a high degree of accuracy, specificity and confidence. In some cases, an aggregation of the multiple measurements using the same affinity reagent (e.g. repeating a binding reaction in triplicate) can allow characterization to be made with a high degree of accuracy, specificity and confidence. Optionally, a plurality of promiscuous reagents can be reacted with a given analyte and the reaction outcome observed for each of the promiscuous reagents can be detected. Promiscuous reagents can demonstrate both low specificity, with regard to the variety of different analytes recognized, and high reactivity for some or all of those analytes. Taking a binding reaction as an example, promiscuous affinity reagents can demonstrate both low specificity, with regard to the variety of different analytes recognized, and high affinity for some or all of those analytes. For any of a variety of reactions, including but not limited to binding reactions, a first reaction carried out using a first promiscuous reagent may perceive a first subset of analytes in a sample without distinguishing one analyte in the subset from another analyte in the sample. A second reaction carried out using a second promiscuous reagent may perceive a second subset of analytes in the sample, again, without distinguishing one analyte from another analyte in the second subset. However, a combination of measurements obtained from the first and second reactions can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the known or suspected diversity of different analytes for a given sample.

A composition, system or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system such as an array, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKₐ for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art. An analyte can be identified with a probability or score surpassing a desired threshold for confident identification.

Methods, compositions and systems of the present disclosure can be deployed in situations where proteins yield different empirical binding profiles despite having identical primary structure and being subjected to the same set of affinity reagents. For example, the methods, compositions and systems are well suited for single-molecule detection and other formats that are prone to stochastic variability. Particular configurations of the compositions, systems and methods herein can overcome ambiguities and errors in observed measurement outcomes (e.g. binding outcomes) to provide accurate identification and characterizations of proteins. The methods can be deployed for complex samples including proteomes or subfractions thereof.

Terms used herein will be understood to take on their ordinary meaning in the relevant art unless specified otherwise. Several terms used herein and their meanings are set forth below.

As used herein, the term “address” refers to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity reagent” refers to a molecule or other substance that is capable of specifically or reproducibly binding to an analyte (e.g. protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof. The term “binding reagent” is intended to be synonymous with the term “affinity reagent.”

As used herein, the term “array” refers to a population of analytes (e.g. proteins) that are associated with unique identifiers such that the analytes can be distinguished from each other. A unique identifier can be, for example, a solid support (e.g. particle or bead), spatial address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the term “outcome profile” refers to a plurality of outcomes for reaction of a given protein or other analytes. The outcomes can be obtained from independent observations of reactions of the given protein with the other analytes, for example, independent binding outcomes acquired using different affinity reagents, respectively. Alternatively, the outcomes can be statistical measures such as probabilities, likelihoods, measures of uncertainty or measures of variation. Outcomes can be generated in silico, for example, being derived from a modification of an empirically obtained outcome. An outcome profile can include empirical measurement outcomes, candidate outcomes, pseudo outcomes, putative outcomes, calculated outcomes, theoretical outcomes or a combination thereof. An outcome profile can exclude one or more of empirical measurement outcomes, candidate outcomes, pseudo outcomes, calculated outcomes, theoretical outcomes or putative outcomes. An outcome profile can include a vector of outcomes. The elements of the vector can be digital values (e.g. binary values representing positive and negative measurement outcomes respectively) or analog values (e.g. probability values in a range from 0 to 1).

As used herein, the term “binding profile” refers to a plurality of binding outcomes for a protein or other analyte. The binding outcomes can be obtained from independent binding observations, for example, independent binding outcomes can be acquired using different affinity reagents, respectively. Alternatively, the outcomes can be statistical measures such as probabilities, likelihoods, measures of uncertainty or measures of variation. Optionally, the binding outcomes can be generated in silico, for example, being derived from a modification of an empirically obtained binding outcome. A binding profile can include empirical measurement outcomes, candidate measurement outcomes, pseudo measurement outcomes, putative measurement outcomes, calculated measurement outcomes, theoretical measurement outcomes or a combination thereof. A binding profile can exclude one or more of empirical measurement outcomes, candidate measurement outcomes, pseudo measurement outcomes, calculated measurement outcomes, or theoretical measurement outcomes or putative measurement outcomes. A binding profile can include a vector of binding outcomes. The elements of the vector can be digital values (e.g. binary values representing positive and negative binding outcomes respectively) or analog values (e.g. probability values in a range from 0 to 1).

As used herein, the term “comprising” is intended to be open-ended, including not only the recited elements, but further encompassing any additional elements.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

As used herein, the term “measurement outcome” refers to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome can include an empirical or putative measurement outcome for a candidate analyte (e.g. for a candidate protein) that is known or suspected of being present in a sample or assay. A “pseudo” measurement outcome can be a measurement outcome that is known or suspected of not being characteristic of any candidate analyte or extant analyte. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

As used herein, the term “promiscuous,” when used in reference to a reagent, means that the reagent is known or suspected to react with a variety of different analytes in a given sample. For example, an affinity reagent that is known or suspected to recognize a variety of different analytes (e.g. a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample.

As used herein, the term “protein” refers to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “single,” when used in reference to an object such as an analyte, means that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

The embodiments set forth below and recited in the claims can be understood in view of the above definitions.

### Protein Assay Configurations

The present disclosure provides a method of identifying an extant protein. The method can include steps of (a) providing inputs to a computer processor, the inputs including: (i) a binding profile, wherein the binding profile includes a plurality of binding outcomes for binding of the extant protein to a plurality of different affinity reagents, wherein individual binding outcomes of the plurality of binding outcomes include a measure of binding between the extant protein and a different affinity reagent of the plurality of different affinity reagents, the binding profile including positive binding outcomes and negative binding outcomes, (ii) a database including information characterizing or identifying a plurality of candidate proteins, and (iii) a binding model for each of the different affinity reagents; (b) determining a probability for each of the affinity reagents binding to each of the candidate proteins in the database according to the binding model; and (c) identifying the extant protein as a selected candidate protein, the selected candidate protein being a candidate protein in the database having a probability for binding each of the affinity reagents that is most compatible with the binding profile for the extant protein. Optionally, the inputs can further include (iv) a non-specific binding rate including a probability of a non-specific binding event occurring for one or more of the different affinity reagents.

Also provided is method of identifying an extant protein, which includes steps of: (a) contacting a plurality of different affinity reagents with a plurality of extant proteins in a sample; (b) acquiring binding data from step (a), wherein the binding data includes a plurality of binding profiles, wherein each of the binding profiles includes a plurality of binding outcomes for binding of an extant protein of step (a) to a plurality of different affinity reagents, wherein individual binding outcomes of the plurality of binding outcomes include a measure of binding between an extant protein of step (a) and a different affinity reagent of the plurality of different affinity reagents, each of the binding profiles including positive binding outcomes and negative binding outcomes; (c) providing a database including information characterizing or identifying a plurality of candidate proteins; (d) providing a binding model for each of the different affinity reagents; (e) determining a probability for each of the affinity reagents binding to each of the candidate proteins in the database according to the binding model; and (f) identifying the extant proteins as selected candidate proteins, the selected candidate proteins being candidate proteins in the database having a probability for binding each of the affinity reagents that is most compatible with the plurality of binding outcomes for the extant proteins.

The methods, compositions and systems of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof. Polymeric analytes including, for example, those composed of a finite set of monomeric structures, albeit in various sequences, are particularly well suited for methods, compositions and systems set forth herein. The monomeric structures can be treated akin to amino acids and the polymers can be treated akin to proteins in the exemplary configurations set forth herein.

One or more proteins used herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or system set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or system set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or system set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or system set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or system set forth herein can have a complexity of at least 2, 5, 10, 100, 1×10³, 1×10⁴, 1×10⁵, 1×10⁶, 1×10⁸, 1×10¹⁰, or more different primary protein structures. Alternatively or additionally, a plurality of proteins can have a complexity that is at most 1×10¹⁰, 1×10⁸, 1×10⁶, 1×10⁵, 5×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or system set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

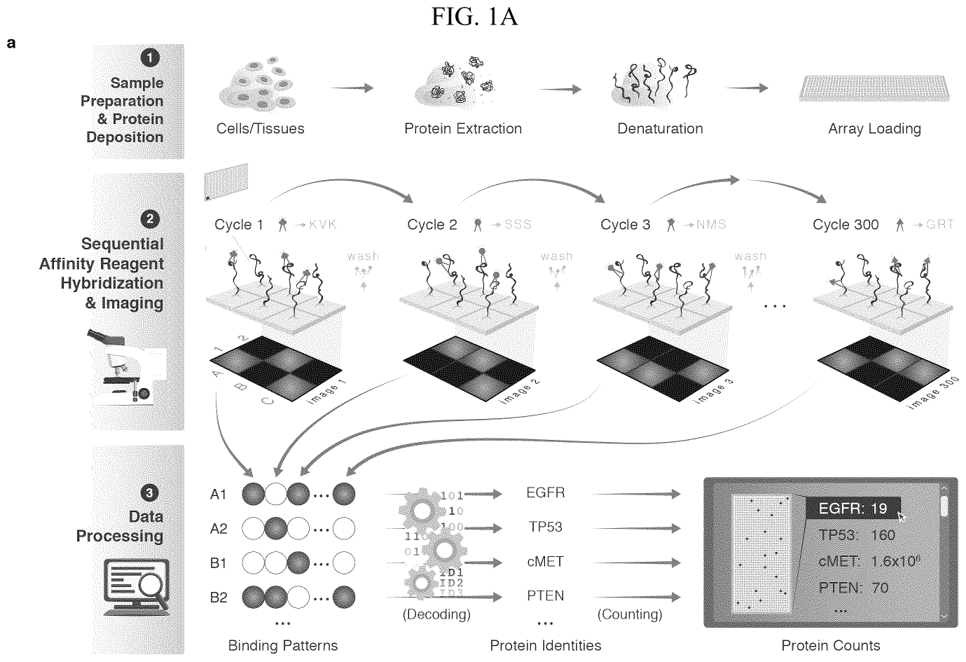

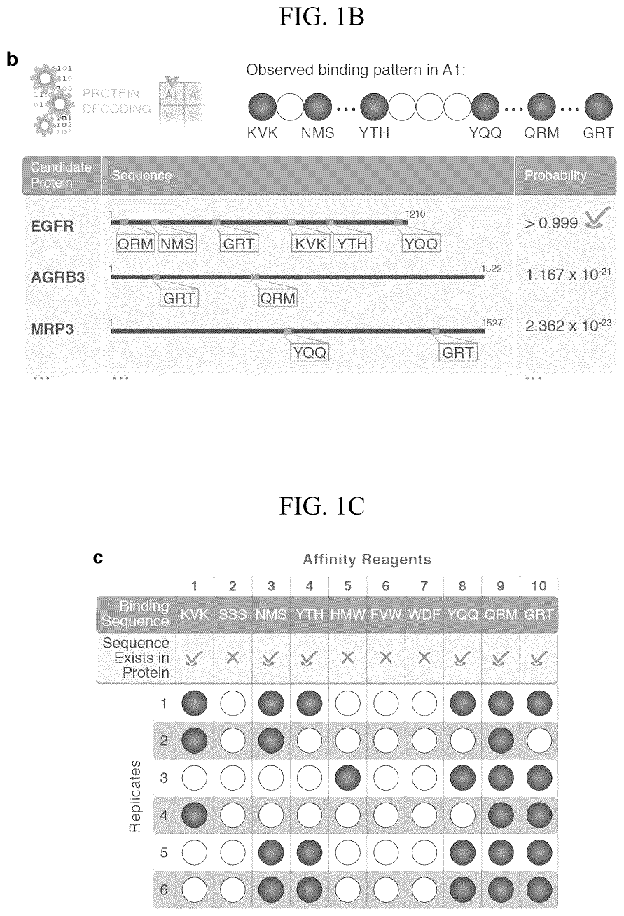

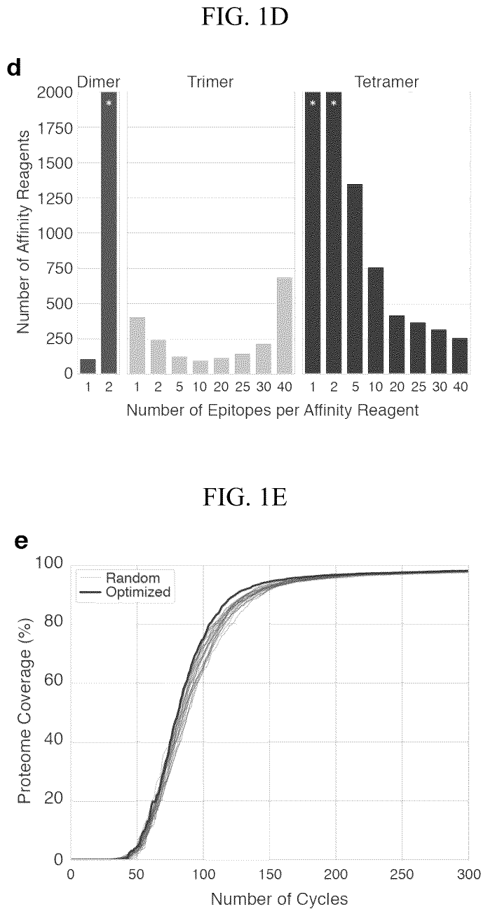

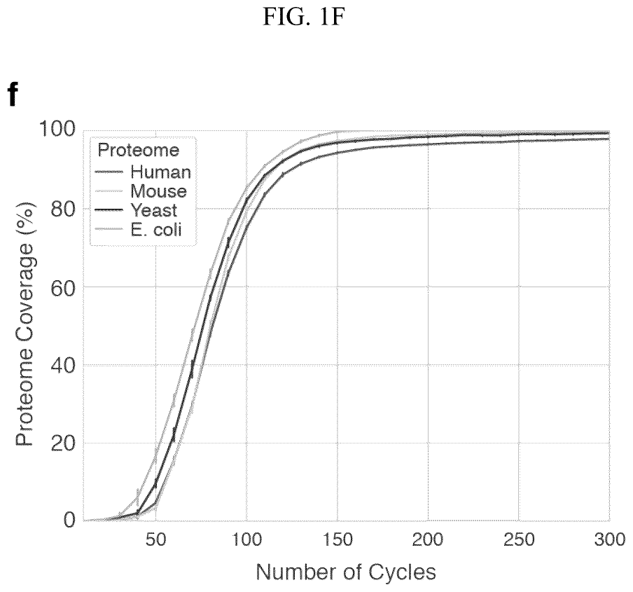

The present disclosure provides assays that are useful for detecting one or more analytes. An exemplary assay format is shown diagrammatically in FIG. 1A. Proteins can be extracted from a sample and attached to an array. Optionally, the unique identifiers of the array can be addresses. The array can be configured to have a plurality of addresses, wherein individual addresses are attached to individual proteins, respectively, from the sample. The proteins that are attached to the array can be in a denatured state or native state. Optionally, a structured nucleic acid particle (SNAP) can mediate attachment of each protein to its respective address. Other linkers or attachment chemistry that can be used additionally or alternatively to SNAPs include, but are not limited to, those set forth in US Pat. App. Pub. No. 2021/0101930 A1, WO 2021/087402 A1, or U.S. Pat. App. Ser. No. 63/159,500, each of which is incorporated herein by reference.

Typically, the identity of the protein at any given address is not known (as such, the proteins may be referred to as ‘unknown’ proteins). Methods set forth herein can be used to identify proteins at one or more addresses in the array. Accordingly, the methods can be used to locate extant proteins in an array. Continuing with the example diagrammed in FIG. 1A, a plurality of affinity reagents (e.g. antibodies, aptamers, or small proteins), tagged with fluorophores, can be contacted with the array, and fluorescence can be detected from individual addresses to determine binding outcomes. The affinity reagents can be delivered to the array and detected serially as shown, such that each cycle detects binding outcomes for an individual affinity reagent. In some configurations of the methods set forth herein, a plurality of different affinity reagents can be delivered in a cycle. The different affinity reagents that are delivered in a given cycle can be configured as a pool of indistinguishably labeled reagents (or they can lack labels), such that the different reagents are not distinguished in the detection step. Alternatively, two or more different affinity reagents that are delivered in a given cycle can be distinguishably labeled. As such the affinity reagents can be distinguishably detected when bound to proteins on the array. The use of fluorescent labels and fluorescent detection is exemplary. Other labels and other detectors can be used such as those set forth herein or known in the art.

Further examples of reagents and techniques that can be used to detect proteins in a method, system or composition of the present disclosure are set forth, for example, in U.S. Pat. No. 10,473,654 or US Pat. App. Pub. Nos. 2020/0318101 A1 or 2020/0286584 A1; or Egertson et al., BioRxiv (2021), DOI: 10.1101/2021.10.11.463967, each of which is incorporated herein by reference. Exemplary methods, systems and compositions are set forth in further detail below.

Some configurations of the compositions, systems or methods set forth herein, can distinguish different proteoforms, such as proteins having the same primary structure (i.e. the same sequence of amino acids) but differing with respect to the number, type, or location of post-translational modifications. Methods of the present disclosure can be configured to identify a number, type, or location for one or more post-translational modifications in one or more proteins of a sample. Exemplary post-translational modifications include, but are not limited to, a phosphoryl, glycosyl (e.g. N-acetylglucosamine or polysialic acid), ubiquitin, acyl (e.g. myristoyl or palmitoyl), isoprenyl, prenyl, farnesyl, geranylgeranyl, lipoyl, acetyl, alkyl (e.g. methyl or ethyl), flavin, heme, phosphopantetheinyl, C-terminal amidation, hydroxyl, nucleotidyl, adenylyl, uridylyl, proprionyl, S-glutathionyl, sulfate, succinyl, carbamyl, carbonyl, SUMOyl, or nitrosyl moiety.

Any of a variety of affinity reagents can be used in a composition, system or method set forth herein. An affinity reagent can be characterized, for example, prior to use in a method set forth herein, with respect to its binding properties. Exemplary binding properties that can be characterized include, but are not limited to, specificity, strength of binding; equilibrium binding constant (e.g. K_{A} or K_{D}); binding rate constant, such as association rate constant (kₒₙ) or dissociation rate constant (koff); binding probability; or the like. Binding properties can be determined with regard to an epitope, a set of epitopes (e.g. a set of proteins having structural similarities), a protein, a set of proteins (e.g. a set of proteins having structural similarities), or a proteome.

An affinity reagent can include a label. Exemplary labels include, without limitation, a fluorophore, luminophore, chromophore, nanoparticle (e.g., gold, silver, carbon nanotubes), heavy atom, radioactive isotope, mass label, charge label, spin label, receptor, ligand, nucleic acid barcode, polypeptide barcode, polysaccharide barcode, or the like. A label can produce any of a variety of detectable signals including, for example, an optical signal such as absorbance of radiation, luminescence (e.g. fluorescence or phosphorescence) emission, luminescence lifetime, luminescence polarization, or the like; Rayleigh and/or Mie scattering; magnetic properties; electrical properties; charge; mass; radioactivity or the like. A label component may produce a signal with a characteristic frequency, intensity, polarity, duration, wavelength, sequence, or fingerprint. A label need not directly produce a signal. For example, a label can bind to a receptor or ligand having a moiety that produces a characteristic signal. Such labels can include, for example, nucleic acids that are encoded with a particular nucleotide sequence, avidin, biotin, non-peptide ligands of known receptors, or the like.

A method set forth herein can be carried out in a fluid phase or on a solid phase. For fluid phase configurations, a fluid containing one or more proteins can be mixed with another fluid containing one or more affinity reagents. For solid phase configurations one or more proteins or affinity reagents can be attached to a solid support. One or more components that will participate in a binding event or other detectable event can be contained in a fluid and the fluid can be delivered to a solid support, the solid support being attached to one or more other component that will participate in the binding event or other detectable event.

A method of the present disclosure can be carried out at single analyte resolution. A single analyte (e.g. a single protein) may be resolved from other analytes based on, for example, spatial or temporal separation from the other analytes. An alternative to single-analyte resolution is ensemble-resolution or bulk-resolution. Bulk-resolution configurations acquire a composite signal from a plurality of different analytes or affinity reagents in a vessel or on a surface. For example, a composite signal can be acquired from a population of different protein-affinity reagent complexes in a well or cuvette, or on a solid support surface, such that individual complexes are not resolved from each other. Ensemble-resolution configurations acquire a composite signal from a first collection of proteins or affinity reagents in a sample, such that the composite signal is distinguishable from signals generated by a second collection of proteins or affinity reagents in the sample. For example, the ensembles can be located at different addresses in an array. Accordingly, the composite signal obtained from each address will be an average of signals from the ensemble yet signals from different addresses can be distinguished from each other.

A composition, system or method set forth herein can be configured to contact one or more proteins (e.g. an array of different proteins) with a plurality of different affinity reagents. For example, a plurality of affinity reagents (whether configured separately or as a pool) may comprise at least 2, 5, 10, 25, 50, 100, 250, 500, 1000 or more types of affinity reagents, each type of affinity reagent differing from the other types with respect to the epitope(s) recognized. Alternatively or additionally, a plurality of affinity reagents may comprise at most 1000, 500, 250, 100, 50, 25, 10, 5, or 2 types of affinity reagents, each type of affinity reagent differing from the other types with respect to the epitope(s) recognized. Different types of affinity reagents in a pool can be uniquely labeled such that the different types can be distinguished from each other. In some configurations, at least two, and up to all, of the different types of affinity reagents in a pool may be indistinguishably labeled. Alternatively or additionally to the use of unique labels, different types of affinity reagents can be delivered and detected serially when evaluating one or more proteins (e.g. in an array).

A method of the present disclosure can be performed for a single analyte (e.g. a single protein gene product) or in a multiplex format. In multiplexed formats in which the analytes are proteins, different proteins that are to be detected can be attached to different unique identifiers (e.g. addresses in an array), and the proteins can be manipulated and detected in parallel. For example, a fluid containing one or more different affinity reagents can be delivered to an array such that the proteins of the array are in simultaneous contact with the affinity reagent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a proteome or proteome subfraction that is analyzed in a method set forth herein can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, presence of unexpected proteins in a source for the sample, or loss of some proteins prior to analysis.

A particularly useful multiplex format uses an array of proteins and/or affinity reagents. A protein can be attached to a unique identifier (e.g. address of an array) using any of a variety of means. The attachment can be covalent or non-covalent. Exemplary covalent attachments include chemical linkers such as those achieved using click chemistry or other linkages known in the art or described in US Pat. App. Pub. No. 2021/0101930 A1, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. at an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in US Pat. App. Pub. No. 2021/0101930 A1, WO 2021/087402 A1, or U.S. Pat. App. Ser. No. 63/159,500, each of which is incorporated herein by reference.

A method of the present disclosure can include a step of assaying binding between a protein and affinity reagent to determine a measurement outcome. For example, the measurement outcome for contacting an affinity reagent with an analyte can be observed as a binding outcome. The binding outcome can be positive or negative. For example, observation of binding is a positive binding outcome and observation of non-binding is a negative binding outcome. A binding outcome can be a null binding outcome, for example, when a positive binding outcome cannot be distinguished from a negative binding outcome.

Binding can be detected using any of a variety of techniques that are appropriate to the reaction components used. For example, binding can be detected by acquiring a signal from a label attached to an affinity reagent when the affinity reagent is bound to an observed protein, acquiring a signal from a label attached to protein when the protein is bound to an observed affinity reagent, or signal(s) from labels attached to an affinity reagent and protein when bound to each other. In some configurations a complex between a protein and affinity reagent need not be directly detected, for example, in formats where a nucleic acid tag or other moiety is created or modified as a result of binding between the protein and affinity reagent. Optical detection techniques such as luminescent intensity detection, luminescence lifetime detection, luminescence polarization detection, or surface plasmon resonance detection can be useful. Other detection techniques include, but are not limited to, electronic detection such as techniques that utilize a field-effect transistor (FET), ion-sensitive FET, or chemically-sensitive FET. Exemplary methods are set forth in U.S. Pat. No. 10,473,654 or U.S. patent application Ser. No. 17/523,869, each of which is incorporated herein by reference.

The present disclosure provides a decoding method, for example, in the form of a decoding process, that can be used to evaluate the results of an assay set forth herein. Decoding will be exemplified herein in the context of an assay that employs one or more binding reaction to detect proteins. Those skilled in the art will recognize that the decoding methods set forth herein can also be applied to assays that employ other types of assay reagents, other types of reactions or other types of analytes. Returning to the example of a protein binding assay, decoding results can be used to identify or otherwise characterize extant proteins. In some configurations, distinct and reproducible binding profiles may be observed for some or even a substantial majority of proteins that are to be identified in a sample. However, in many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed individually. The present disclosure provides decoding methods that provide accurate protein identification despite ambiguities and imperfections that can arise in single-molecule formats or other contexts.

Methods for identifying or characterizing analytes in a sample can utilize a decoding method that analyzes an empirical outcome profile acquired for a plurality of reactions carried out between each analyte in the sample and a plurality of assay reagents, and then the empirical outcome profile can be evaluated with respect to known or predicted interactions of the assay reagents with a plurality of candidate analytes. In some configurations, methods for identifying or characterizing one or more extant proteins in a sample utilize a decoding method that analyzes an empirical binding profile acquired for a plurality of binding reactions carried out between each extant protein in the sample and a plurality of affinity reagents, and then the empirical binding profile is evaluated with respect to the binding behavior of the affinity reagents to a plurality of candidate proteins. The plurality of candidate proteins can include proteins that are known or suspected of being present in the sample. Thus, the plurality of candidate proteins can include a plurality of native amino acid sequences. The decoding process can output the identity of the extant protein as the candidate protein that has binding characteristics most compatible with the empirical binding profile. This compatibility can be determined based on a binding model that represents the affinity of each of the candidate proteins for each of the affinity reagents that were used to produce the empirical binding profile. A strong candidate protein can be identified as one for which the modeled binding outcomes are more consistent with the empirical binding profile as compared to the other candidate proteins evaluated.

A decoding method can be configured to evaluate positive outcomes with or without evaluating negative outcomes. For example, a decoding method can be configured to evaluate positive binding outcomes. In a censored decode configuration, the decoding method can evaluate positive binding outcomes without evaluating negative binding outcomes. In a non-censored decode configuration, a strong candidate protein can be identified as one for which a combination of positive binding outcomes and negative binding outcomes is more consistent with the empirical binding profile as compared to the other candidate proteins evaluated. A candidate protein can be identified as weak or even incorrect based on having many instances where positive binding outcomes and/or negative binding outcomes are inconsistent with the empirical binding profile being evaluated. The strongest candidate protein can be deemed the most likely identity for the extant protein and confidence in this identification can be computed as a relative measure of the compatibility of the most likely protein compared to all of the other candidate proteins.

A computer processor can be configured to execute a decoding method that outputs identities for one or more extant proteins (or other analytes) based on various inputs. A particularly useful input is empirical assay data, for example, empirical binding data for binding of an extant protein to a plurality of different affinity reagents. The binding data can be in the form of an empirical binding profile that includes a plurality of empirically observed binding outcomes. An empirical binding profile can include positive binding outcomes or negative binding outcomes. The same can be true for a candidate outcome profile or pseudo outcome profile. In some configurations a binding profile will include both positive binding outcomes and negative binding outcomes. For example, decoding can be carried out in an ‘uncensored’ configuration, wherein both positive and negative outcomes (e.g. binding outcomes) are considered. Alternatively, decoding can be carried out in a ‘censored’ configuration, wherein a subset of outcomes or a particular type of outcome is not considered. For example, a censored configuration can consider positive binding outcomes and omit negative binding outcomes. A censored approach can be useful, for example, in situations where there is an expectation that particular binding measurements or binding outcomes are prone to an unacceptable or undesirable level of errors or artifacts.

The present disclosure provides a ‘semi-censored’ decoding configuration, wherein positive and negative outcomes are evaluated independent of each other. Looking to the example of binding assays, an uncensored configuration can be used to compute negative binding outcomes as the difference of one minus the positive binding outcome. For a semi-censored configuration negative binding probabilities can be computed independent of computing positive binding probabilities. Semi-censored configurations provide a distinct method for updating protein likelihood from negative binding outcomes in comparison to the method used for positive binding outcomes. In a semi-censored configuration, positive binding outcomes can be weighted more heavily relative to negative binding outcomes. Alternatively, negative binding outcomes can be weighted more heavily relative to positive binding outcomes in a semi-censored configuration. The different weights can be applied to offset an expected or suspected bias in the binding reactions being evaluated, such as a high rate of off-target binding by one or more affinity reagents.

An empirical outcome profile can be input to a decoding method set forth herein. For example, an empirical binding profile can be input to a computer processor that performs the decoding method. A series of empirical binding outcomes that constitute an empirical binding profile can be acquired using binding reactions such as those set forth herein or known in the art. Alternatively, a binding profile can be obtained from a simulation and used similarly to an empirical binding profile. Each empirical binding outcome in a binding profile can result from one binding reaction among a plurality of binding reactions carried out between an extant protein and a plurality of affinity reagents. An empirical binding profile can be decoded after all binding outcomes have been acquired for a given extant protein. Alternatively, for example, when binding outcomes are acquired serially, decoding can occur in real time such that evaluation of an empirical binding outcome from an earlier binding reaction in the series is initiated, and perhaps completed, prior to, or during, acquisition of an empirical binding outcome for a subsequent binding reaction in the series. A plurality of empirical binding outcomes need not necessarily be acquired serially, for example, instead being acquired such that some or all binding outcomes in an empirical binding profile are acquired from binding reactions that occur in parallel.

Another useful input to a decoding method is information for a plurality of candidate analytes, such as candidate proteins. For example, a database of candidate protein information can be input to a computer processor that performs the decoding method. A plurality of candidate proteins may include at least 10, 25, 50, 75, 100, 500, 1×10³, 1×10⁴, 1×10⁶, 1×10⁸ or more different candidate proteins. In some cases, a complete proteome or substantial fraction thereof can be included. For example, a database can include at least 10%, 25%, 50%, 75%, 90%, 95%, 99% or more of the proteins known, or suspected, to be present in a proteome set forth herein or known in the art. A database may include candidate proteins from more than one organism. For example, a database can include organisms from a given ecosystem such as a microbiome or environmental sample, organisms from a particular family, class or genera of species; or all known proteins from all known species. In some embodiments, primary structures (i.e. amino acid sequences), secondary structures, tertiary structures, quaternary structures, names, or other information pertaining to the candidate proteins can be stored in a database. Particularly useful information that can be included in a database includes, for example, binding characteristics for binding of one or more affinity reagents to a protein. However, such information need not be included and can instead be provided by a binding model. For example, the information can include a probability for each of a plurality of affinity reagents binding to each of a plurality of candidate proteins. In some configurations, such binding probabilities or other binding characteristics are derived empirically, for example, from binding experiments carried out between one or more known candidate proteins and one or more known affinity reagent(s). In some embodiments, binding probabilities or other binding characteristics are derived based on a priori information such as presence of a suspected epitope sequence in the primary structure (e.g. amino acid sequence) of a candidate protein. Any of a variety of publicly available databases can be used, such as those set forth in Example I, herein.

A database can include a probability or likelihood that a candidate analyte would generate a positive outcome. For example, a database can include a probability or likelihood that a candidate protein would generate a positive binding outcome. Such information can be useful for several decoding configurations including, for example, censored, uncensored or semi-censored configurations. A database can further include a probability or likelihood that a candidate analyte would generate a negative outcome. For example, a database can further include a probability or likelihood that a candidate protein would generate a negative binding outcome. Such information can be useful for an uncensored or semi-censored decoding configuration.

A binding model can be input to a decoding method set forth herein. For example, the binding model can be input to a computer processor that performs the decoding method. Optionally, a binding model can include a function for determining probability of a specific binding event occurring between a protein and each of a plurality of affinity reagents. In some configurations, a binding model can include a function for determining probability of a specific binding event occurring between a protein epitope and each of a plurality of affinity reagents. Epitopes evaluated by the model can have any of a variety of characteristics of interest. For example, the epitopes can have a defined length (e.g. the epitope length being less than or equal to 2, 3, 4, 5 or 6 amino acids in a protein primary sequence) or chemical composition (e.g. sequence of amino acids in a protein primary sequence). In some cases, the chemical composition can be relatively general with regard to chemical characteristics of amino acid side chains (or other moieties) such as charge, polarity, hydropathy, steric size, steric shape or the like. For example, the chemical composition of an epitope can be expressed in terms of biosimilarity to another epitope.

A decoding method set forth herein can include a function for calculating a probability of each assay reagent reacting with some or all possible candidate analytes among a plurality of candidate analytes in a given database. For example, a decoding method set forth herein can include a function for calculating a probability of each affinity reagent binding to some or all possible candidate proteins among a plurality of candidate proteins in a given database. The function can consider positive binding outcomes. Optionally, the function can further consider negative binding outcomes, for example, when the function is used in an uncensored or semi-censored configuration. Optionally, binding probabilities can be configured as a matrix. As demonstrated in Example I, positive binding outcomes can be included in an M×N binding probability matrix B. In an uncensored configuration, the probability of a probe not binding to a protein can be expressed as: P(affinity probe not binding protein)=1−P(affinity probe binding protein). When using a binding probability matrix, a non-binding probability matrix U can be calculated as U=1−B. However, the uncensored approach may be adversely impacted by one or more non-binding events having an outsized impact on decoding. For example, an affinity reagent may not bind to a specific site for numerous difficult-to-predict reasons (e.g., protein structure, presence of unexpected post-translational modifications that hinder binding, etc.).

A decoding method set forth herein can include a function for determining probability of a non-specific binding event occurring between a protein and a plurality of affinity reagents. The model can account for the context of one or more epitopes in a given candidate protein. For example, a function for determining probability can be normalized with respect to the length of the given candidate protein. Alternatively or additionally, a binding model used in a method or system set forth herein can include a function for determining probability of a specific binding event occurring between a candidate protein and each of the affinity reagents. Again, the model can account for the context of one or more epitopes in a given candidate protein. For example, the function can be normalized with respect to the length of the given candidate protein.

In some configurations, a decoding method can include a function for determining probability of a binding event occurring between each of the affinity reagents and an epitope that is biosimilar to a specific epitope for the respective affinity reagent. In a biosimilar model, an affinity reagent can be considered as targeting a specific epitope to which it binds with a particular probability. For example, the probability can be at least 0.01, 0.05, 0.1, 0.25 0.5, 0.75, 0.9, 0.99 or higher. Alternatively or additionally, the probability can be at most 0.99, 0.9, 0.75, 0.5, 0.25, 0.1, 0.05, 0.01 or lower. The affinity reagent can also be considered to bind one or more additional primary off targets with a probability in a range above. The number of additional primary targets can be at least 1, 3, 5, 7, 9, 15, 20 or more epitopes that are biosimilar to the targeted epitope. Alternatively or additionally, the number of additional primary targets can be at most, 20, 15, 9, 7, 5, 3 or 1 epitopes that are biosimilar to the targeted epitope. Biosimilar epitope targets can be selected by computing a pairwise similarity score of the target epitope to every other possible epitope of the same length and then selecting one or more of the other epitopes with a high similarity score. A similarity score can be computed by summing up similarity between the pair of residues at each sequence location, for example, using BLOSUM62 or other function for determining biosimilarity.

A parameterized binding model can be used in a decoding method of the present disclosure. For example, an affinity reagent can be modeled by assigning a binding probability to each unique target epitope recognized by the affinity reagent. In another example, an affinity reagent can be modeled by assigning a binding probability to each candidate protein recognized by the affinity reagent. Optionally, a non-specific binding rate can be assigned to individual affinity reagents. The non-specific binding rate can, for example, represent probability of a given affinity reagent binding to any epitope in a protein non-specifically (or to any candidate protein non-specifically). The probability of an affinity reagent binding to a given candidate protein can be computed by first computing the probability of a specific binding event happening. The model can consider the count of each epitope in a given protein sequence. The binding model parameters can include a vector of probabilities of a given affinity reagent binding to each recognized epitope (or to each candidate protein). Furthermore, the model can include a function for computing the probability of a non-specific protein binding event happening. Optionally, the model can take into account the length of each candidate protein sequence, the length of an epitope recognized by the affinity reagent or both. The probability of the affinity reagent binding to the protein and generating a detectable signal can be represented as the probability of one or more specific or non-specific binding events occurring. Exemplary binding models are provided in Example I herein.

In some configurations of a system or method set forth herein, a non-specific reaction rate can be provided as an input. Turning to the example of a binding assay, a non-specific binding rate can be provided as an input. The input can be in the form of one fixed non-specific binding rate for all affinity reagents, or a unique non-specific binding rate for each affinity reagent. Also, non-specific binding rate can be learned iteratively and/or adaptively in the same manner as other parameters in an affinity reagent binding model. The non-specific binding event can be binding of an affinity reagent to a substance other than a protein. The substance can be a solid support attached to an extant protein. For example, a non-specific binding event can occur at a region of an array where no protein of interest resides, such as a location at or near an address where a protein of interest resides. In some cases, a non-specific binding event can occur at an empty address, where a protein does not reside or at an interstitial region on the array that separates one address from another. Optionally, as exemplified in Example I herein, the input can be a surface non-specific binding rate describing the probability of a surface non-specific binding event happening in any given cycle in a series of binding reactions.

Execution of a decoding process can include computing a probability matrix that includes the probabilities of a positive outcome, which is exemplified below in terms of binding outcomes for individual affinity reagents binding to each candidate protein used in a binding reaction. Optionally, the method can further include computing a probability matrix that includes the probabilities of a negative binding outcome for individual affinity reagents binding to each candidate protein used in a binding reaction. For example, adjusted non-binding probabilities can be computed as set forth in Example I, herein. In an alternative configuration of systems and methods set forth herein, the probabilities of a negative binding outcome can be calculated by subtracting the probabilities of a positive binding outcome from 1, the probabilities being represented by a value between 0 and 1. Positive and negative binding outcomes can be equally weighted. Alternatively, positive binding outcomes can be weighted more heavily relative to negative binding outcomes. In other cases, negative binding outcomes can be weighted more heavily relative to positive binding outcomes. The latter weighting can be particularly desirable to account for the numerous difficult-to-predict mechanisms by which an affinity reagent may bind to proteins non-specifically.

Decoding can be carried out by computing a vector of likelihoods for a plurality of candidate analytes, such as candidate proteins. The candidate protein of highest likelihood can be selected. For example, the selected candidate protein can be the one having the most probabilities for binding the affinity reagents that are consistent with most of the binding outcomes obtained for a given extant protein. In another example, a candidate protein can be selected by multiplying the probabilities of the observed binding outcomes. Optionally, if there was a tie for top protein, one of the top proteins can be selected randomly or by another desired criteria. The probability of an identification being correct can be based on the likelihood of the top protein being correct divided by the sum of the likelihood of all other candidate proteins being correct. The protein identity can be output from the decoding system or method. Optionally, the probability of an identification being correct can be output. The probability can be calculated as the quotient of dividing the likelihood of a selected candidate protein by the sum of the likelihoods determined for all the other candidate proteins that were evaluated by the decoding process.

Exemplary processes, and methods for characterizing proteins that can be used in combination with a method or system set forth herein include, for example, those set forth in US Pat App. Pub. No 2020/0286584 A1 or Egertson et al., BioRxiv (2021), DOI: 10.1101/2021.10.11.463967, each of which is incorporated herein by reference.

A decoding method can output information pertaining to the identity for one or more analytes, as exemplified for extant proteins. The information output for a given protein can be in the form of a determined identity for the protein or in the form of a probability or likelihood for one or more identity of the protein. For example, the most likely identity for an extant protein, the likelihood or probability of the extant protein having a particular identity, or both can be output by a decoding method. A decoding method can output a non-digital or non-binary score for the identity of a given extant protein or for the likelihood of the extant protein having a particular identity. For example, probability or likelihood scores can be output in the form of an analog value between 0 and 1, or percent value between 0% and 100%. In some configurations, a digital or binary score that indicates one of two discrete states can be output to indicate the identity of a protein or at least a subset of proteins (e.g. a family of proteins sharing a common structural motif) to which the protein belongs.

### Quality Assessment: Pseudo Profiles

The present disclosure provides methods for determining uncertainty or variation of protein characterizations, such as protein identifications made by a decoding method or e. Methods for evaluating uncertainty or variability are exemplified herein in the context of protein identification based on results of a binding assay but can be applied to other types of assays and other types of analytes. A method of identifying extant proteins can be configured to determine a false discovery statistic that is indicative of the accuracy of the protein identifications. A false discovery statistic for an assay can be determined based on the observed propensity of a decoding process to mis-identify extant analytes as pseudo analytes that are known to be absent from the assay. For example, a false discovery statistic for a protein identification assay can be determined based on the observed propensity of a decoding process to mis-identify extant proteins as pseudo proteins that are known to be absent from the assay. Generally, a false discovery statistic that is determined or used as set forth herein can provide a global measure of uncertainty or variability. Whether or not accuracy can be individually determined for a given protein, a global indication of uncertainty or variability can provide useful information that informs subsequent analysis or conclusions drawn from a protein assay. Based on this information analysis methods or parameters used by the methods can be modified and applied to assay results to produce more accurate results. Furthermore, experimental protocols can be changed in view of the information, thereby improving accuracy for the same or similar sample when subjected to the changed experimental protocol.

A method for determining uncertainty or variation of protein characterization can include steps of (a) providing the following inputs to a computer processor: (i) a plurality of empirical outcome profiles, individual empirical outcome profiles of the plurality of empirical outcome profiles each including a plurality of empirical measurement outcomes for an extant protein in a sample, (ii) a plurality of candidate outcome profiles, individual candidate outcome profiles of the plurality of candidate outcome profiles each including a plurality of statistical measures for a candidate protein of a plurality of candidate proteins, wherein the candidate proteins are known or suspected of being present in the sample, and (iii) a plurality of pseudo outcome profiles, individual pseudo outcome profiles of the plurality of pseudo outcome profiles each including a plurality of statistical measures that is known to not occur for any of the candidate proteins; (b) performing a process in the computer processor to identify extant proteins of the plurality of different extant proteins based on the empirical outcome profiles of the extant proteins and the plurality of candidate outcome profiles; and (c) performing a process in the computer processor to determine a false discovery statistic for the extant proteins based on the plurality of empirical outcome profiles and the plurality of pseudo outcome profiles.

In some configurations, a method for determining uncertainty or variation of protein characterization can include steps of: (a) contacting an array of different extant proteins with a plurality of different affinity reagents, wherein individual addresses of the array are each attached to an extant protein; (b) acquiring empirical binding profiles from the individual addresses, the empirical binding profiles each including a plurality of binding outcomes for binding of an extant protein at one of the individual addresses to the plurality of different affinity reagents; (c) providing a plurality of candidate outcome profiles, individual candidate outcome profiles of the plurality of candidate outcome profiles each including a plurality of statistical measures for a candidate protein of a plurality of candidate proteins, wherein the candidate proteins are known or suspected of being present in the sample; (d) providing a plurality of pseudo outcome profiles, individual pseudo outcome profiles of the plurality of pseudo outcome profiles each including a plurality of statistical measures that is known to not occur for any of the candidate proteins; (e) identifying extant proteins of the array based on the empirical binding profiles of the extant proteins and the plurality of candidate outcome profiles; and (f) determining a false discovery statistic for the extant proteins based on the empirical binding profiles of the extant proteins and the plurality of pseudo outcome profiles.

A method for determining uncertainty or variation of protein characterization can employ empirical outcome profiles as set forth herein, for example, in the context of methods for identifying proteins. For example, a plurality of empirical outcome profiles can include individual empirical outcome profiles that each include a plurality of empirical measurement outcomes for an extant protein, and individual empirical measurement outcomes of the plurality of empirical measurement outcomes can each include a measured outcome for reaction of the extant protein with a different assay reagent.

A method for determining uncertainty or variation of protein characterization can employ candidate outcome profiles as set forth herein, for example, in the context of methods for identifying proteins. For example, a plurality of candidate outcome profiles can include individual candidate outcome profiles that each include a plurality of statistical measures for a candidate protein, individual statistical measures of the plurality of statistical measures including a measure of uncertainty or variation for reaction of the candidate protein with a given assay reagent.

A method for determining uncertainty or variation of protein characterization can employ pseudo outcome profiles. A pseudo outcome profile can include a plurality of statistical measures that is known or suspected to not occur for any protein in a sample of interest. As such, methods that utilize a plurality of candidate outcome profiles, wherein the candidate proteins are known or suspected of being present in a sample of interest, can employ pseudo outcome profiles that are distinct from any and all of the candidate outcome profiles. However, pseudo outcome profiles can be related to candidate outcome profiles. For example, a pseudo outcome profile can be paired with a given candidate protein by virtue of the pseudo outcome profile being generated by modification of the candidate outcome profile for the given candidate protein. A plurality of pseudo outcome profiles can be related to a plurality of candidate outcome profiles by one or more function set forth herein in the context of generating pseudo outcome profiles from candidate outcome profiles. For example, a plurality of pseudo outcome profiles can include vectors that are rearranged, digitized, or subtracted from a plurality of candidate outcome vectors. In some cases, pseudo outcome profiles can be produced from application of a mapping function to candidate outcome vectors.

Several different approaches can be used to generate pseudo outcome profiles. A sequence-centric approach can be used whereby (1) amino acid sequences for pseudo proteins are generated based on the amino acid sequences of candidate proteins and (2) pseudo outcome profiles are generated from the pseudo protein sequences. In this approach, sequence characteristics of a plurality of candidate proteins can be compared, clustered or classified in a variety of ways, examples of which are set forth below, and pseudo sequences can be generated based on modifications of the candidate protein sequences. A sequence-agnostic approach can be used in which pseudo outcome profiles are generated from candidate outcome profiles. In this approach, pseudo outcome profiles can be generated by modification of candidate outcome profiles, for example, via mathematic or logic functions such as those exemplified below.

A pseudo outcome profile can be thought of as representing a pseudo protein, wherein the pseudo protein is contrived. In a sequence-centric approach, a pseudo outcome profile can be generated from an amino acid sequence, such as an amino acid sequence that is contrived or otherwise known not to be present in a particular assay. However, the amino acid sequence for a pseudo protein need not be known nor does the sequence or any other biochemical property of the pseudo protein need to be derivable from the pseudo outcome profile. Accordingly, pseudo outcome profiles can be generated by modifying outcome profiles for candidate proteins using a sequence-agnostic approach. Whether employing a sequence-centric or sequence-agnostic approach to generate pseudo outcome profiles, some or all candidate outcome profiles in a database can have a paired pseudo outcome profile. For example, a given candidate outcome profile can be paired with a single pseudo outcome profile. Alternatively, a given candidate outcome profile can be paired with a plurality of pseudo outcome profiles. Optionally, a given pseudo outcome profile can be paired with a single candidate outcome profile or with a plurality of candidate outcome profiles. A pairing can arise from a pseudo outcome profile having been generated by modification of a candidate outcome profile, for example, using a sequence-agnostic approach. The modifications that are made to a plurality of candidate outcome profiles can be configured to generate pseudo outcome profiles that are similar enough to the candidate outcome profiles to serve as decoys for a protein identification assay. In a sequence-centric approach, a pairing can arise from a pseudo outcome profile having been generated from the amino acid sequence of a pseudo protein. The propensity of an assay to identify decoys (e.g. pseudo outcome profiles or pseudo proteins) rather than candidate proteins can provide a measure of quality for the assay.

In a sequence-centric approach to generating pseudo outcome profiles, amino acid sequences for a plurality of candidate proteins can be evaluated to identify pairs or other groups of candidate proteins that have similar sequences. The next closest protein (NCP) for a first candidate protein can be identified as the candidate protein having the amino acid sequence that is closest to the amino acid sequence for the first candidate protein. Closeness of amino acid sequences can be determined using sequence comparison processes known in the art such as sequence clustering methods. Particularly useful processes include, for example, Smith-Waterman (Smith & Waterman, J. Mol. Bio. 147: 195-197 (1981)), Needleman-Wunsch (Needleman & Wunsch. J. Mol. Bio. 48: 443-53 (1970)), or MMseqs2 (Steinegger and Soding Nat. Biotech. 35:1026-1028 (2017)). The foregoing citations are incorporated herein by reference. Amino acid sequences can be aligned using a bidirectional approach. In an exemplary configuration of the bidirectional approach, if a first sequence contains at least 70% of a second sequence, then the first protein is clustered with the second protein and the second protein is clustered with the first protein. This approach can be applied to a plurality of candidate sequences to assign the candidate sequences to a plurality of clusters. The use of 70% sequence overlap is exemplary. It will be understood that other overlaps can be used to provide different pseudo outcome profiles including, for example, at least 50%, 60%, 80%, 90% or higher. Another approach for aligning amino acid sequences is a target-in-query approach. In an exemplary configuration, if a first sequence contains no more than 60% of a second sequence, but the second sequence contains no more than 70% of the first sequence, then the first protein is clustered with the second protein but the second protein is not clustered with the first protein. Again, the sequence overlaps are exemplary and can be adjusted to obtain different sets of pseudo outcome profiles. For example, clusters may be formed if the first sequence contains no more than 25%, 50%, 75% or 90% of the second sequence, but the second sequence contains no more than 25%, 50%, 75% or 90% of the first sequence. The bidirectional and target-in-query approaches can be applied to a plurality of candidate sequences to assign the candidate sequences to a plurality of clusters. Candidate proteins that are in the same cluster as a first candidate protein can be treated as NCPs to the first candidate protein. The candidate outcome vector for the first candidate protein or a candidate outcome vector for at least one NCP in the same cluster can be modified to generate a pseudo outcome profile that is paired with the first candidate protein.

A pseudo protein can be generated to have an amino acid sequence that is equidistant from the amino acid sequences of a given candidate protein and its next closest candidate protein. For example, a BLOSUM62 substitution matrix can be used to generate pseudo protein sequences from candidate protein sequences. As this example demonstrates, pseudo proteins can be generated based on comparison of amino acid sequences that are resolved at the level of individual amino acid residues. In alternative configurations, amino acid sequences can be compared or generated at a resolution of dimers, trimers, tetramers, pentamers or other amino acid sequence multimers. This configuration can be useful when evaluating results of a protein binding assay in which the length of the multimers being compared matches the length of the epitopes that are known or suspected to be bound by affinity reagents used in the assay. Looking to the example of a binding assay in which affinity reagents recognize trimer epitopes, candidate proteins can be represented as sets of trimers and closeness of the candidate proteins can be evaluated in terms of the closeness of the sets of trimers. A pseudo protein can be identified as an amino acid sequence having a set of trimers that is equidistant from the trimer sets of a given candidate protein and its next closest candidate protein. A pseudo protein can be generated from a multimer-based substitution matrix such as a dimer-based substitution matrix, trimer-based substation matrix, tetramer-based substation matrix, pentamer-based substation matrix, or hexamer-based substation matrix, etc. Optionally, a multimer-based substitution matrix can be weighted to account for known or suspected probability of an affinity reagent binding to particular multimer epitopes in the matrix.

In some configurations of the present methods, candidate outcome profiles can be selected for use in generating pseudo outcome profiles based on any of several different criteria. A set of candidate outcome profiles, for example in a database, can be evaluated for amino acid sequence similarity (e.g. at the resolution of monomers or multimers). Depending upon the sequence alignment process used and the cutoffs used for sequence overlap, not all candidate sequences may cluster with at least one other candidate sequence. The candidate outcome profiles for candidate proteins that successfully cluster can proceed to pseudo outcome profile generation. The candidate outcome profiles for candidate proteins that do not cluster can be evaluated with respect to distance to other candidate outcome profiles, and can then proceed to pseudo outcome profile generation. In this example, candidate proteins having sequences that do not cluster with at least one other candidate sequence are rejected. It will be understood that other cutoff values can be applied, such as a requirement for a candidate sequence to cluster with at least 2, 3, 4, 5, 10 or more candidate sequences. Optionally, multiple pseudo profiles can be generated by applying the results of the amino acid similarity and distance to other approaches set forth herein for modifying candidate outcome profiles.

In some cases, the amino acid sequence for a pseudo protein can be identified from a source in nature other than the source from which extant proteins under assay are to be derived. For example, an assay that is used to characterize proteins from a first organism can utilize proteins from a second organism as pseudo proteins. The second organism can be selected based on evolutionary distance from the first organism or other criteria that select a proteome that is divergent from the proteome of the first organism. The amino acid sequence for a pseudo protein can be generated by modifying the amino acid sequence of a candidate protein. Exemplary modifications include, but are not limited to rearrangement of one or more amino acids, shuffling of sequence regions (e.g. the sequence regions can be epitopes used in a binding assay), reversal of the amino acid sequence, reversal of the sequence for one or more sequence regions, replacement of one or more amino acids, removal of one or more amino acids, addition of one or more amino acids, or a combination thereof. A candidate protein that is to be subjected to sequence modification can be known or suspected of being present in a given assay or in a source for the extant proteins under assay. Alternatively, the candidate protein that is to be modified can be from a source other than the source from which the extant proteins are derived. The amino acid sequence of a pseudo protein can be generated from a simulation such as a trained Markov model (e.g. a hidden Markov model), neural network or generative-adversarial neural net.

Turning to sequence-agnostic approaches, any of a variety of criteria can be used to select candidate outcome profiles for use in generating pseudo outcome profiles. A particularly useful criteria is the distance between candidate outcome profiles. Distance between outcome profiles can be illustrated for candidate outcome profiles that are configured as vectors. In this case, distance between vectors can be determined from a string metric such as the Hamming distance, Euclidian distance, Levenshtein distance, Sørensen-Dice coefficient, block distance (e.g. L1 distance or city block distance), Jaro-Winkler distance, simple matching coefficient, Jaccard similarity, overlap coefficient, Hellinger distance, information radius (i.e. Jensen-Shannon divergence), Kullback-Leibler divergence, or Tau metric. A method set forth herein can be configured to evaluate closeness of a first candidate outcome vector to a plurality of other candidate outcome vectors. Optionally, no more than a single pseudo outcome profile is generated per candidate outcome profile. Alternatively, a plurality of pseudo outcome profiles can be generated per candidate outcome profile.

Pseudo outcome profiles can be generated by modifying candidate outcome profiles. This can be the case whether a sequence-centric or sequence agnostic approach is used to identify candidate outcome profiles to be modified. Exemplary modifications include, but are not limited to, rearrangement of a candidate outcome profile, mathematical manipulation of numerical values in a candidate outcome profile, logical manipulation of symbols in a candidate outcome profile or a combination thereof. For purposes of illustration, and without intending to be limited by the illustration, a candidate outcome profile can be represented as a vector of binding outcomes for binding of a plurality of different affinity reagents to the candidate protein. The vector of binding outcomes can include binding probability values.

A candidate outcome vector that is to be modified in a method set forth herein can include binding outcomes that are analog in nature, such as non-binary values indicating a measured or predicted binding probability. Optionally, a method of generating a pseudo outcome profile can include a step of converting binding probabilities to digital values. For example, binding probabilities above a threshold value can be assigned a first binary value (e.g. 1) and binding probabilities below the threshold being assigned a second binary value (e.g. 0). In the case where binding probabilities are measured on a scale from 0 to 1, a threshold can be, for example, at least 0.05, 0.1, 0.2, 0.3, 0.4, 0.5, 0.6, 0.7, 0.8, 0.9 or higher. Alternatively or additionally, the threshold can be at most 0.9, 0.8, 0.7, 0.6, 0.5, 0.4, 0.3, 0.2, 0.1, 0.05 or lower. The use of digitized values can provide for more efficient computation. On the other hand, using a profile having analog probabilities rather than digitized outcomes may be more appropriate for stochastic single molecule measurements. Binding outcomes in a vector can be any of those exemplified herein including, for example, analog probability values, or probability values in binary, ternary or other radix.

A given threshold can be applied to all elements of a given matrix. For example, given threshold can be applied to the binding probabilities for all affinity reagents for all candidate outcome vectors in a given matrix. Alternatively, threshold values can differ for respective vectors in a given matrix. Returning to the example of a binding assay, threshold values can differ on a ‘per affinity reagent’ basis (e.g. threshold values are the same for all elements of a given affinity reagent vector, whereas different affinity reagent vectors can have different threshold values) or on a ‘per candidate protein’ basis (e.g. threshold values are the same for all elements of a given candidate protein vector, whereas different candidate protein vectors have different threshold values). An exemplary method that can be used for thresholding binding probabilities is Otsu's method (Otsu, IEEE Trans. Sys. Man. Cyber. 9: 62-66 (1979), which is incorporated herein by reference). Any of a variety of multi-level classification methods can be used to determine threshold values including, for example, a supervised learning method such as a mixture model (e.g. Gaussian mixture model), or an unsupervised learning model such as k-means clustering (Lloyd, IEEE Transactions on Information Theory 28:129-137 (1982), which is incorporated herein by reference). Clustering methods can partition the data into at least 2, 3, 4, 5, 10 or more clusters. Alternatively or additionally, data can be partitioned into at most 10, 5, 4, 3 or 2 clusters. A desired number of clusters can be determined using Bayesian Information Criteria, Akaike Information Criteria or Silhouette Score.

A method of generating a pseudo outcome profile can include a step of rearranging the order of elements in a vector. Taking the example of a vector in which the elements are binding outcomes, the rearrangement can be a shuffled order for some or all binding outcomes with respect to the order of the different affinity reagents in the vector. The rearrangement can be a reversed order for some or all elements in the vector, such as a reverse order for some or all binding outcomes with respect to the order of the different affinity reagents in the vector. As such, the order for some or all binding outcomes with respect to the order of the different affinity reagents can be reversed in a pseudo outcome vector compared to the candidate outcome vector from which it was derived. In another configuration, the rearrangement can be a shuffled order for one or more strings of elements in a vector, such as a shuffled order for binding outcomes with respect to the order of the different affinity reagents in the vector. As such, the order for some or all binding outcomes with respect to the order of the different affinity reagents can be shuffled in a pseudo outcome vector compared to the candidate outcome vector from which it was derived. A pseudo outcome profile can be said to be paired with the candidate outcome profile from which it is generated. In some cases, a partial shuffle can occur. For example, shuffling can occur for only positive binding outcomes or alternatively for only negative binding outcomes. Exemplary methods for rearranging outcome vectors are set forth in Example II, herein.

A method of generating a pseudo outcome profile can include a step of performing a mathematical function on the elements in a candidate outcome vector. For example, a modified vector can be generated by calculating the difference between a vector of binding outcomes for one candidate protein and the vector of binding outcomes for a second candidate protein. Optionally, an outcome vector for a first candidate protein can be subtracted from an outcome vector for the candidate protein that is the next closest protein (NCP) of the first candidate protein. The result of the subtraction can be a pseudo outcome profile that is coupled with the candidate outcome vector for the first candidate protein. Conversely, a pseudo outcome profile that is coupled with the candidate outcome vector for the first candidate protein can be generated by subtracting a candidate outcome vector for the NCP from the candidate outcome vector for the first candidate protein. Exemplary methods for performing mathematical functions on outcome vectors are set forth in Example II, herein. It will be understood that a pseudo outcome vector can be generated by a combination of modifications set forth herein. Example II describes modification of candidate outcome vectors by converting analog probability values to digital values, subtracting a first digital candidate outcome vector from a second digital candidate outcome vector to produce a difference vector, and rearranging the difference vector to produce a pseudo outcome vector. In another option, a pseudo outcome profile can be generated by randomizing a candidate outcome vector. For example, an outcome vector for a first candidate protein can be generated by randomizing the outcome vector for the candidate protein that is the next closest protein (NCP) of the first candidate protein. It will be understood that the manipulations set forth above can be carried out in different orders to suit a particular application of the methods set forth herein.

A pseudo outcome profile can be generated by calculating the difference between an outcome vector for a first candidate protein and an outcome vector for a second candidate protein, and adding the resulting difference vector to the vector for the first candidate protein. The candidate protein vectors can be configured to have integer elements, for example, selected from 0, 1, 2, 3, 4, 5, 10 or more. Taking the example of a vector having four integer elements, the elements of the difference vector will include integers in the range of −4 to 4. Optionally, addition of the difference vector to the first candidate vector only allows (i) elements having a value −1 to be added to elements having the value 1, and (ii) elements having the value 1 to be added to elements having the value 0. In a first alternative approach, the addition of a difference vector to a first candidate vector can occur between all elements irrespective of the values for the elements. However, the values resulting from the vector addition can be capped at a particular value. Returning to the example where vector elements are selected from 0, 1, 2, 3 or 4, all values less than 0 can be assigned a value of 0, and all values greater than 4 can be assigned a value of 4. In a second alternative approach, the addition of a difference vector to the first candidate vector can be limited to only allow (i) negative values to be added to positive values and (ii) positive values to be added to 0 values. In a third alternative approach, the addition of a difference vector to the first candidate vector can be limited to only sum to values within a range. Returning to the example where vector elements are selected from 0, 1, 2, 3 or 4, the range can be 0 to 4. In this case a value of 3 can only be added with a value of 1, 0, −1, −2 or −3 (i.e. not 2, 3, 4 or −4). Similarly, a value of 2 can only be added with a value of 2, 1, 0, −1, or −2 (i.e. not 3, 4, −4 or −3). A fourth alternative is to treat the difference vector as a transition vector. For example, the presence of a 2 to 0 transition from the first candidate protein vector to a paired candidate protein vector can cause an element having the value of 2 to be changed to a value of 0 for the pseudo outcome vector. In a configuration wherein 300 affinity reagents are used, 300 transitions are applied to the pseudo outcome vector.

Pseudo outcome profiles can optionally be generated using a kernel density estimate method. Exemplifying this approach in the context of a binding assay, a distribution of binding outcomes can be obtained from the outcome profiles for a first candidate protein and its next closest candidate protein, and a pseudo outcome profile can be generated from the distribution. For example, a scatter plot can be obtained by plotting the outcomes for binding of a series of affinity reagents with the first candidate protein vs. the outcomes for binding of the series of affinity reagents with its next closest candidate protein. The scatter plot can then be converted to a contour plot using a kernel density estimate conversion. A slice through the contours at any given value of x or y that corresponds to a given affinity reagent, can be sampled to generate pseudo binding probabilities for the affinity reagent.

A plurality of pseudo proteins or pseudo outcome profiles can be input to a decoding method. For example, a plurality of pseudo proteins or pseudo outcome profiles can be present in a database. Optionally, the database can also include a plurality of candidate proteins or candidate outcome profiles. Pseudo proteins can be paired with candidate proteins such that a plurality of pseudo proteins that is present in a database or used herein can include one or more pseudo proteins per candidate protein. Similarly, pseudo outcome profiles can be paired with candidate outcome profiles such that a plurality of pseudo outcome profiles that is present in a database or used herein can include one or more pseudo outcome profiles per candidate outcome profile. For example, each candidate outcome profile can be paired with at least 1, 2, 3, 4, 5, 10, 25, 50, 100, 250 or more pseudo outcome profiles. Alternatively or additionally, each candidate outcome profile can be paired with at most 250, 100, 50, 25, 10, 5, 4, 3, 2, or 1 pseudo outcome profiles. Optionally, all candidate outcome profiles can be paired with at least one pseudo outcome profile. Alternatively, one or more candidate outcome profiles need not be paired with a pseudo outcome profile.

The number of pseudo outcome profiles used for determining false detection statistics can be increased, for example, to improve the quality assessment of a given assay. In some cases, the number of pseudo outcome profiles can be relatively low, for example, to reduce computation costs. Accordingly, the number of pseudo proteins or pseudo outcome profiles present in a database or used in a method set forth herein can be the same, less than or greater than the number of candidate proteins or candidate outcome profiles present in the database or used in the method. For example, a plurality of pseudo proteins may include at least 10, 25, 50, 75, 100, 500, 1×10³, 1×10⁴, 1×10⁶, 1×10⁸ or more different pseudo proteins. A plurality of pseudo outcome profiles can include a similar range of binding profiles.

Optionally, the number of pseudo outcome profile used to evaluate assay results can by dynamic, for example, being modified based on empirically observed assay results. For example, the number of candidate outcome profiles that are paired with at least one pseudo outcome profile can by dynamic, for example, being modified based on empirically observed assay results. Similarly, the number of pseudo outcome profiles that are paired with a given candidate outcome profile can be modified based on empirically observed assay results, as can the average number of pseudo outcome profiles that are paired per candidate outcome profile. This dynamic determination can be responsive to available computational resources or time. Increased resources or time can allow use of more pseudo outcome profiles. Another basis that can be used for deciding how many pseudo outcome profiles to employ is the apparent certainty of decoding results. For example, fewer pseudo outcome profiles can suffice if the false discovery statistics are relatively certain, but as uncertainty increases the number of pseudo outcome profiles can be dynamically increased. Conversely, if certainty of false discovery statistics increase, then the number of pseudo outcome profiles can be dynamically decreased.

In some configurations, a decoding method can be performed to evaluate an empirical binding profile with respect to a plurality of pseudo outcome profiles. A plurality of individual empirical binding profiles can each be evaluated with respect to pseudo outcome profiles of the plurality of pseudo proteins. Each pseudo outcome profile in the plurality of pseudo outcome profiles can be paired with a respective candidate outcome profile. As such, a given candidate protein can be considered to be paired with a pseudo protein. Decoding can be carried out in a configuration wherein an empirical binding profile is evaluated with respect to a combined set of binding profiles that includes both candidate outcome profiles and respectively paired pseudo outcome profiles. In this configuration, the candidate outcome profiles can be stored in a database where pseudo outcome profiles are also sorted. Alternatively, separate decoding processes can be carried out for candidate outcome profiles and pseudo outcome profiles. For example, in a decoding process each individual empirical binding profile can be evaluated with respect to a set of candidate outcome profiles, absent any pseudo outcome profiles. In another decoding process, each individual empirical binding profile can be evaluated with respect to a set of pseudo outcome profiles, absent any candidate outcome profiles. Optionally, the candidate outcome profiles can be stored in a database that is separate from the database where pseudo outcome profiles are stored. If desired, a hybrid decoding approach can be used whereby (i) a first subset of empirical binding profiles is evaluated with respect to a combined set of binding profiles that includes both candidate outcome profiles and respectively paired pseudo outcome profiles, and (ii) a second subset of empirical binding profiles are subjected to separate decoding processes for candidate outcome profiles and pseudo outcome profiles, respectively. The subsets of empirical binding profiles can be dynamically adjusted for repeated decoding rounds, for example, to iteratively arrive at an improved assay result.

Decoding can use pseudo outcome profiles in a censored configuration or non-censored configuration. A censored decoding method can be configured to evaluate positive binding outcomes for empirical binding profiles, candidate outcome profiles and/or pseudo outcome profiles, without consideration of negative binding outcomes. A non-censored decoding method can be configured to evaluate both positive binding outcomes and negative binding outcomes for empirical binding profiles, candidate outcome profiles and/or pseudo outcome profiles. As a further option, decoding can use empirical binding profiles, candidate outcome profiles and/or pseudo outcome profiles in a ‘semi-censored’ configuration.

A database can include a probability or likelihood that a pseudo protein would generate a positive binding outcome. Such information can be useful for several decoding configurations including, for example, censored, uncensored or semi-censored configurations. A database can further include a probability or likelihood that a pseudo protein would generate a negative binding outcome. Such information can be useful for an uncensored or semi-censored decoding configuration. Optionally, the probability or likelihood of binding can be specified for a particular affinity reagent, and stored as a list, matrix or other plurality of probabilities or likelihoods.

Execution of a decoding process can include computing a probability matrix that includes the probabilities of a positive outcomes for pseudo analytes, for example, positive binding outcome for individual affinity reagents binding to each of a plurality of pseudo proteins. Optionally, the method can further include computing a probability matrix that includes the probabilities of a negative outcomes for pseudo analytes, for example, negative binding outcome for individual affinity reagents binding to each pseudo protein. For example, adjusted non-binding probabilities can be computed as set forth in Example II, herein. Positive and negative binding outcomes in a pseudo outcome profile can be equally weighted due to equal weighting being used for binding outcomes in a candidate outcome profile from which the pseudo outcome profile is derived. Alternatively, positive binding outcomes can be weighted more heavily relative to negative binding outcomes in a pseudo outcome profile. In other cases, negative binding outcomes can be weighted more heavily relative to positive binding outcomes. Weighted outcomes can be present in a pseudo outcome profile, for example, when derived from weighted binding outcomes in a candidate outcome profile.

Decoding can be carried out by computing a vector of likelihoods for a plurality of candidate analytes (e.g. candidate proteins) and a vector of likelihoods for a plurality of pseudo analytes (e.g. pseudo proteins). The candidate analyte or pseudo analyte of highest likelihood in view of empirical outcomes from an assay can be selected. For example, the selected candidate protein or pseudo protein can be the one having the most probabilities for binding the affinity reagents that are consistent with most of the binding outcomes obtained for a given extant protein. In another example, a candidate protein or pseudo protein can be selected by multiplying the probabilities of the empirical binding outcomes. The probability of an identification being correct can be based on the likelihood of the top candidate or pseudo protein divided by the sum of the likelihood of all other candidate and/or pseudo proteins being correct. In the event that a candidate protein is identified, the protein identity can be output from the decoding system or method. In some cases, a plurality of two or more different candidate protein identities can be output. Optionally, the probability for one or more identifications being correct can be output. A probability can be calculated as the quotient of dividing the likelihood of a selected candidate protein by the sum of the likelihoods determined for all the other candidate proteins that were evaluated by the decoding process. In the event that a pseudo protein is identified, the protein can be indicated as being mis-identified in an output from the decoding system or method.

A method of the present disclosure, such as a method for determining uncertainty or variation of protein characterization, can be configured to determine a false discovery statistic that is indicative of the accuracy of the protein characterization. A particularly useful false discovery statistic is the false identification rate (also referred to herein as the “false discovery rate”). The false identification rate (FIR) assesses the fraction of false positive identifications on a per extant protein basis. Taking the example of evaluating array-based data, the FIR can assess the fraction of false positives per unique identifier (e.g. per address). The false identification rate can be used to conceptualize the rate of type I errors in null hypothesis testing when comparing multiple comparisons, a type I error being the mistaken rejection of an actually true null hypothesis or ‘false positive.’ For example, a false identification rate can assess all unique identifiers (e.g. addresses) in an array for which a protein identification has been made by identifying the fraction (or percent) of the unique identifiers in which the identification is incorrect. In a first option, false identification rate can be determined from the following equation: FIR=([number of incorrect identifications]+1)/[number of unique identifiers]. In a second option, false identification rate can be determined from the following equation: FIR=(([number of incorrect identifications]*2)+1)/[number of unique identifiers]. A false identification distribution can be used to evaluate skew or bias in the distribution of addresses in an array that are incorrectly identified. Another useful false discovery statistic is the false detection rate (FDR). The false detection rate assesses whether a candidate protein is detected in an assay. The FDR can be used in a protein identification method to provide an estimate of the fraction (or percent) of the identified proteins that are incorrectly identified. A false detection distribution can be used to estimate skew or bias in the distribution of protein identifications over a plurality of proteins evaluated.

A false discovery statistic can be determined across a full set of unique identifiers (e.g. addresses) or proteins evaluated in a method set forth herein. Alternatively, a subset of evaluated unique identifiers (e.g. addresses) or candidate proteins can be considered, for example, the subset having a decode score above or below a given threshold, the subset having a probability or likelihood of being a particular candidate protein that is above or below a given probability threshold or likelihood threshold, the subset identified as being a particular candidate protein, or the subset identified as having a particular characteristic such as having a particular amino acid sequence motif, being expressed from a particular gene or being in a particular protein family. A subset of evaluated unique identifiers (e.g. addresses) or candidate proteins can be selected based on a set of candidate proteins that are of interest, for example, to answer a research or clinical question. A set of candidate proteins of interest can be, for example, proteins involved in a biochemical pathway such as a metabolic pathway, catabolic pathway, anabolic pathway, signal transduction pathway, developmental pathway, necrosis pathway, apoptosis pathway, protein expression pathway, protein maturation pathway, protein secretion pathway, DNA replication pathway, DNA transcription pathway, mRNA translation pathway, immunological response, or the like. A set of candidate proteins of interest can also be determined based on a set of proteins expected to be obtained fractionation of a sample, including for example, a subcellular fraction such as a membrane fraction or cytosolic fraction; an organelle such as a mitochondria, endoplasmic reticulum, Golgi apparatus, nucleus or chloroplast; a tissue type; a cell type or the like.

In some configurations, a false discovery statistic can include, or can be used to derive, an expectation, or e-value. The expectation can represent how many random matches would be expected to achieve a given score or greater, in a search of a given size. Optionally, a false discovery statistic can include or can be used to derive a threshold value for accuracy of protein identifications, or to derive a distribution of accuracies. A false discovery statistic can include or can be used to derive a signal to background ratio for input of data into a decoding process. The process can be iterative, whereby an initial signal to background value is used for decoding, the decoding results are evaluated according to desired parameters and based on the results, an adjusted signal to background value is used for the decoding process. Further iterations can be used to arrive at a refined signal to background value.

A false discovery statistic for an analyte assay can be determined based on the observed propensity of a decoding process to mis-identify one or more extant analytes as pseudo analytes that are known to be absent from the assay. For example, a false discovery statistic for a protein identification assay can be determined based on the observed propensity of a decoding process to mis-identify one or more extant proteins as pseudo proteins that are known to be absent from the assay. A false discovery statistic for a protein identification assay can be determined based on the observed propensity of a decoding process to determine that empirical binding profiles have greater compatibility with pseudo outcome profiles compared to candidate outcome profiles. Typically, a false discovery statistic is output or evaluated as an aggregate statistic. For example, a false detection rate may be used to determine that, among a set of protein identifications a particular fraction are false positives based on the fraction of empirical binding profiles that are more compatible with a pseudo outcome profile than with a candidate outcome profile. A collection of unique identifiers (e.g. addresses) or protein identifications found to have a high fractional rate of compatibility with pseudo outcome profiles may be considered to have a high number of false identifications. The aggregate statistics can be useful even if the accuracy of each individual protein identification or of each unique identifier cannot be determined from the analysis.

### Quality Assessment: Entropy

The present disclosure provides methods for assessing quality for characterizations derived from analyte assays. Quality can be assessed in terms of accuracy of results or confidence in the results. For clarity of explanation, the methods are exemplified herein in the context of identifying proteins, but can be applied to any of a variety of other analytes and moreover can be applied to any of a variety of characterizations other than identification, per se. The quality of a protein identification can be evaluated in terms of uncertainty in the identification. Quality (e.g. accuracy or confidence) can be quantified according to an inverse relationship with uncertainty. In this regard, quality increases as uncertainty decreases. A particularly useful measure of uncertainty is information entropy. Information entropy can be expressed and evaluated mathematically and can thus provide a quantifiable measure of the accuracy or confidence for identifications derived from a protein assay.

Information entropy for an identification can be determined from the following equation:

$$H ( X ) = {\sum }_{i} - p ( {x}_{i} ) {log}_{2} ( p ( {x}_{i} ) )$$

wherein H(X) is the information entropy for identification X, p(x) is the probability of the identification, and Σ denotes the sum over the possible values for x. The value of p(X) decreases to a limit of 0 as certainty decreases. Accordingly, H(X) decreases as certainty increases toward a limit of 0 for maximum certainty in an identification.

Information entropy can be used to evaluate convergence of assay results to a confident identification. It can provide a quantitative basis for evaluating certainty in assay results, for example, in the form of numerical values that are useful for comparing identifications made within an assay or between two or more assays. Changes in information entropy can be represented graphically to provide a useful tool for visual evaluation of assay results as they converge to a confident identification. As such, information entropy can provide a useful measure for evaluating variables that impact the certainty of assay results. Exemplary variables that can be evaluated include, but are not limited to, choice of affinity reagents (or reagents for other assays) to include in an assay, sequential order for delivering affinity reagents (or other assay reagents) in an assay, choice of conditions used for the assay (e.g. temperature, ionic strength, pH, duration, etc.), choice of methodologies used to prepare samples for assay, choice of sample sources for proteins (or other analytes) to be assayed, or the like. Information entropy can also provide a useful measure for comparing results within an assay (e.g. comparing identification of a test protein to identification of a control proteins), comparing results between similar assays (e.g. comparing identifications resulting from assays performed using different detection systems or different reagent lots), or comparing results obtained from different assay platforms (e.g. comparing identifications from a protein binding assay to identifications from an Edman-type sequencing assay).

The present disclosure provides a method for identifying a protein, including steps of: (a) inputting to a computer processor measurement outcomes for reactions of a plurality of assay reagents to an extant protein; (b) inputting to the computer processor a database including a plurality of candidate proteins; and (c) in the computer processor: (i) adding a measurement outcome of step (a) to an outcome profile of the extant protein; (ii) determining a collection of probabilities for each of the candidate proteins in the database producing the outcome profile; (iii) determining information entropy for the collection of probabilities; and (iv) repeating steps (i) through (iii). Optionally, the method can be performed with analytes other than proteins.

The above method is exemplified herein for protein binding assays. Accordingly, the method can include steps of: (a) inputting to a computer processor binding outcomes for binding of a plurality of affinity reagents to an extant protein; (b) inputting to the computer processor a database including a plurality of candidate proteins; (c) inputting to the computer processor a binding model for each of the different affinity reagents; and (d) in the computer processor: (i) adding a binding outcome of step (a) to a binding profile of the extant protein; (ii) evaluating the binding model to determine a collection of probabilities for each of the candidate proteins in the database producing the binding profile; (iii) determining information entropy for the collection of probabilities; and (iv) repeating steps (i) through (iii).

The present disclosure provides a method for conducting a protein assay, including the steps of: (a) contacting an array of different extant proteins with assay reagents, wherein individual addresses of the array are each attached to a single extant protein of the different extant proteins; (b) determining a measurement outcome for reaction of the assay reagents at each of the individual addresses of the array; (c) providing a database including a plurality of candidate proteins; and (d) for an individual address in the array: (i) adding a measurement outcome of step (b) to an outcome profile of the individual address; (ii) determining a collection of probabilities for each of the candidate proteins in the database producing the outcome profile; (iii) determining information entropy for the collection of probabilities; and (iv) repeating steps (i) through (iii). Optionally, the method can be performed with analytes other than proteins.

The above method can be particularly useful when configured to conduct a protein binding assay. For example, the method can include steps of (a) contacting an array of different extant proteins with a plurality of different affinity reagents, wherein individual addresses of the array are each attached to a single extant protein of the different extant proteins, and wherein the different affinity reagents recognize different extant proteins in the array; (b) determining a binding outcome for each of the different affinity reagents at each of the individual addresses of the array; (c) providing a database including a plurality of candidate proteins; (d) providing a binding model for each of the different affinity reagents; and (e) for an individual address in the array: (i) adding a binding outcome of step (b) to a binding profile of the individual address; (ii) evaluating the binding model to determine a collection of probabilities for each of the candidate proteins in the database producing the binding profile; (iii) determining information entropy for the collection of probabilities; and (iv) repeating steps (i) through (iii).

Information entropy can be determined or evaluated with respect to a unique identifier that is associated with a sample or analyte of interest. For example, a protein can be associated with an address in an array, an assay can be performed to collect signals from the address, and the signals can be processed to generate information about the protein. In this example, a measure of information entropy for data generated from the address can be used to determine or evaluate the certainty of the data. In a case where the data pertains to the identity of the protein at the address, the information entropy can provide a measure of the certainty for the identification made. As demonstrated by this example, information entropy can provide a measure of quality (e.g. confidence or accuracy) for information collected from an individual unique identifier. This can be the case when the unique identifier is the sole identifier observed in the assay, for example, in a single-plex assay, or when the unique identifier is resolved from other unique identifiers, for example in a multiplex assay.

Information entropy can provide a measure of quality (e.g. confidence or accuracy) for information collected from a plurality of individual unique identifiers. For example, a multiplex assay can be configured to acquire signals from an array of addresses, each of the addresses being attached to a protein, and the signals can be processed to generate information about the protein at each address individually. As set forth above, information entropy can be measured for the addresses individually. However, information entropy for a plurality of unique identifiers can be determined or evaluated collectively. The plurality of unique identifiers can include a subset of the unique identifiers assayed in a multiplex format. Returning to the example of a protein array, information entropy can be evaluated for a subset of addresses that are identified as being attached to the same candidate protein. As such, the information entropy can provide a measure of quality for identification of the candidate protein in the assay as a whole. In some cases, information entropy can be evaluated for a plurality of unique identifiers whether or not they have a common identity. For example, information entropy can be evaluated for substantially all addresses in an array, or for a subset of addresses encompassing a variety of different protein identities. The subset of addresses can be selected based on a set of proteins that are of interest, for example, to answer a research or clinical question. A set of proteins of interest can be, for example, proteins involved in a biochemical pathway such as a metabolic pathway, catabolic pathway, anabolic pathway, signal transduction pathway, developmental pathway, necrosis pathway, apoptosis pathway, protein expression pathway, protein maturation pathway, protein secretion pathway, DNA replication pathway, DNA transcription pathway, mRNA translation pathway, immunological response, or the like. A set of proteins of interest can also be determined based on fractionation of a sample, including for example, a subcellular fraction such as a membrane fraction or cytosolic fraction; an organelle such as a mitochondria, endoplasmic reticulum, Golgi apparatus, nucleus or chloroplast; a tissue type; a cell type or the like.

Optionally, a method of the present disclosure can be configured to evaluate a change in the information entropy measured for two or more detection events in an assay. For example, the detection events can occur in a separate steps that occur serially as different assay reagents (e.g. affinity reagents) are delivered to an analyte (e.g. protein) under assay. In some configurations, a method can include steps of (i) adding a measurement outcome to an outcome profile for an analyte detected at a unique identifier; (ii) determining a collection of probabilities for candidate proteins producing the outcome profile; (iii) determining information entropy for the collection of probabilities at the unique identifier; (iv) repeating steps (i) through (iii); and (v) identifying a change in the information entropy over two or more repeats of steps (i) through (iii). Turning to the example of a binding assay performed for an array of proteins, a method can include steps of (i) adding a binding outcome to a binding profile for an extant protein detected at an individual address; (ii) evaluating a binding model to determine a collection of probabilities for candidate proteins producing the binding profile; (iii) determining information entropy for the collection of probabilities at the individual address; (iv) repeating steps (i) through (iii); and (v) identifying a change in the information entropy over two or more repeats of steps (i) through (iii). A change that is identified for information entropy can be a decrease in the information entropy (e.g. a decrease in H(X) toward 0) or an increase in the information entropy (e.g. an increase in H(X) greater than 0). Optionally, a change in information entropy can be measured or identified in terms of an acceleration or deceleration in the rate of change.

For an assay that is configured to detect interactions of an analyte (e.g. a protein) with a plurality of different assay reagents (e.g. binding reagents), the data acquired from detection of each interaction can cause a change in information entropy for the cumulative assay results. For example, information entropy can change as measurement outcomes (e.g. binding outcomes) are added to an outcome profile (e.g. binding profile). Accordingly, a change in information entropy can be used to evaluate the efficacy of an assay reagent. For example, an affinity reagent can be evaluated in terms of its ability to reduce information entropy or in terms of the magnitude for a reduction in the information entropy. Affinity reagents that reduce information entropy can be selected over those that do not. Alternatively, affinity reagents that do not decrease information entropy, such as those that increase information entropy, can be rejected. Optionally, affinity reagents can be selected based on causing a larger reduction in information entropy compared to other affinity reagents. Alternatively, affinity reagents can be rejected based on causing a smaller reduction in information entropy compared to other affinity reagents. Optionally, results can be evaluated in real time and used to modify or adjust the set of affinity reagents that is to be delivered to an assay. The results can be reported to a user who has the option to modify or adjust which affinity reagents are delivered, what order the affinity reagents are delivered or the number of times a given affinity reagent is delivered (e.g. 2, 3 or more replicates). In some cases, a detection system is configured to automatically make such modifications or adjustments, without necessarily involving a user. As an alternative or addition to evaluating empirical assay results, a change in information entropy can be simulated to predict efficacy of an assay reagent. Accordingly, a change in information entropy can be simulated or predicted for a given assay reagent and the assay reagent can be utilized in a detection method based on the results.

A change in assay conditions can result in a change in information entropy for data generated by the assay. Optionally, such changes can be effected in real time during a given assay or they can be effected in a subsequent assay. Exemplary changes in conditions include, but are not limited to, changes in temperature, ionic strength, pH, duration of contact between analyte and assay reagent, duration of detection, detector gain, intensity of radiation or other energy impinging a sample, presence or absence of auxiliary reagents, solvent polarity or the like. Modifications to conditions can be evaluated in terms of ability to reduce information entropy or in terms of the magnitude of the reduction in the information entropy. Conditions that reduce information entropy can be selected over those that do not. Alternatively, conditions that do not reduce information entropy, such as conditions that increase information entropy, can be rejected. Optionally, conditions can be selected based on the ability to cause a larger reduction in information entropy compared to other conditions. Alternatively, conditions can be rejected based on causing a smaller reduction in information entropy compared to other conditions. As an alternative or addition to evaluating empirical assay results, a change in information entropy can be simulated to predict desirability of a particular condition. Accordingly, a change in information entropy can be simulated or predicted for a given assay condition and the condition can be utilized in a detection method based on the results.

Information entropy can change due to repetition of an assay or repetition of particular steps of the assay. For example, a first measure of information entropy can be generated from detection of an affinity reagent with a protein and a change in the information entropy can result from detecting the interaction a second time, or from removing the affinity reagent and delivering a second aliquot of the affinity reagent to the protein and detecting the resulting interaction. First and second detection events can differ, for example, due to stochastic variability which can be common to single-molecule detection formats. Multiple detection events can also differ when using promiscuous affinity reagents. Accordingly, a change in information entropy can be used to evaluate the benefit of repeated use and/or repeated detection of an assay reagent. For example, repeated use or detection of an assay reagent can be evaluated in terms the ability to reduce information entropy or in terms of reducing information entropy by a given magnitude. If desired, delivery of an assay reagent can be repeated and the conditions for the assay can be changed as well. Alternatively, repeated delivery can be carried out under the same assay conditions as before. As an alternative or addition to evaluating empirical assay results, a change in information entropy can be simulated to predict the effectiveness of repeated delivery and/or detection of a given assay reagent. Accordingly, a change in information entropy can be simulated or predicted for a repeated step and the step can be repeated in a detection method based on the results.

For detection methods that utilize several different assay reagents, the magnitude or direction for changes in information entropy can differ based on the order in which their interactions with the analyte are analyzed. The order of analysis can reflect, for example, the order in which the assay reagents are contacted with an analyte or the order in which their interactions with the analyte are detected. As such, information entropy can be evaluated to determine a desired order for serial delivery and/or detection of assay reagents. The evaluation can focus on changes in information entropy that occur for a given unique identifier, such as a given address in an array; for a subset of unique identifiers, such as addresses in an array that are identified as being associated with the same candidate protein, or for substantially all unique identifiers in an assay. For example, the order in which a plurality of affinity reagents is delivered to an array of proteins can be shuffled to achieve a desired rate of convergence for identification of a particular candidate protein at a subset of addresses that are identified as being attached to the candidate protein. Alternately, the order can be selected to achieve a desired rate of convergence for most or all addresses in an array.

A change in methodology used to prepare samples for assay can result in a change in information entropy for data generated by the assay. Exemplary changes in sample preparation include, but are not limited to, changes in equipment, reagent lot, cell lysis techniques, precipitation techniques, extraction techniques, chromatography techniques, redox state, pH, ionic strength, solvent polarity, amount of the biological source used, state of health for the biological source, environmental state for the biological source, cell or tissue type used as a source, or the like. Modifications to sample preparation methodology can be evaluated in terms of ability to reduce information entropy or in terms of the magnitude of the reduction in the information entropy. Methodologies that reduce information entropy can be selected over those that do not. Alternatively, methodologies that do not reduce information entropy, such as conditions that increase information entropy, can be rejected. Optionally, methodologies can be selected based on the ability to cause a larger reduction in information entropy compared to other methodologies. Alternatively, methodologies can be rejected based on causing a smaller reduction in information entropy compared to other conditions.

Optionally, information entropy can be evaluated to determine the number of detection events to analyze. Turning to the example of an assay that interrogates a protein with a series of affinity reagents, the number of affinity reagent interactions to analyze can be determined based on the information entropy resulting from analysis of one or more additional affinity reagents. For example, analysis can be discontinued when information entropy achieves a threshold level or when the rate of change in the information entropy reaches a threshold rate. Similarly, the shape in a curve of information entropy vs. affinity reagent analyzed can be used as a basis for the decision. The spread of entropy values over two or more unique identifiers can be used to determine when to discontinue analysis. For example, analysis of assay reagents can be discontinued when the spread is reduced to a desired threshold. Discontinuing data analysis can provide the benefit of efficient use of computational resources, for example, freeing up the resources for other uses. Data analysis can be discontinued by a user who makes the above determinations or by an automated system that is configured to receive information entropy data, and continue or discontinue the analysis based on the data.

Optionally, information entropy can be evaluated to determine when to discontinue an assay. Turning again to the example of an assay that interrogates a protein with a series of affinity reagents, the results of the assay can be evaluated in real time. As such, information entropy can be measured prior to delivery of all affinity reagents. In this case, delivery of affinity reagents can be discontinued when information entropy achieves a threshold level or when the rate of change in the information entropy reaches a threshold level. Similarly, the shape in a curve of information entropy vs. affinity reagent analyzed can be used as a basis for the decision. The spread of entropy values over two or more unique identifiers can be used to determine when to discontinue an assay. For example, delivery of assay reagents can be discontinued when the spread is reduced to a desired threshold. Discontinuing reagent delivery can reduce reagent costs, saving time and minimizing waste. An assay can be discontinued by a user who makes the above determinations or by an automated system that is configured to receive information entropy data, and continue or discontinue the assay based on the data.

A method set forth herein can be configured to output a measure of information entropy. The output can be received by a user, such as a lab technician or clinician, or by a computer system, such as a computer that is configured to carry out secondary analysis of assay data. An output can be configured to communicate results or observations set forth herein in the context of measuring or evaluating information entropy. For example, a measure of information entropy for a unique identifier, candidate protein or array of unique identifiers can be output. Optionally, a measure of change in information entropy can be output, for example, as a measure of change across different detection events or across different reagent deliveries. A measure of informational entropy can be output as a number (e.g. a positive number, or a number between 1 and 0), a graphical display of changes in information entropy, or any other convenient format.

Information entropy can be used to plan an assay. For an assay that is configured to identify different proteins in a proteome, the initial measure of information entropy for identifying each protein will be proportional to the complexity of the proteome (i.e. the number of different proteins in the proteome). Generally, as the complexity of the proteome increases, the number of potential identities for any randomly selected protein increases. As such, information entropy for any given address in an array of proteins will typically increase with increase in the complexity of the proteome. The number of assay reagents to use in an assay and the order of their delivery can be determined based on the initial measure of information entropy for the proteome. If desired, results can be simulated and trends in the measure of information entropy (e.g. the rate of change for the information entropy) can be evaluated to plan the assay.

### Additional Protein Assay Configurations

Methods of evaluating uncertainty and variation are exemplified herein in the context of protein binding assays. It will be understood that other protein assays can be used instead and the results can be evaluated for uncertainty and variation. Many protein detection methods, such as enzyme linked immunosorbent assay (ELISA), achieve high-confidence characterization of one or more protein in a sample by exploiting high specificity binding of antibodies, aptamers or other binding agents to the protein(s) and detecting the binding event while ignoring all other proteins in the sample. ELISA is generally carried out at low plex scale (e.g. from one to a hundred different proteins detected in parallel or in succession) but can be used at higher plexity. ELISA methods can be carried out by detecting immobilized binding agents and/or proteins in multiwell plates, on arrays, or on particles in microfluidic devices. Exemplary plate-based methods include, for example, the MULTI-ARRAY technology commercialized by MesoScale Diagnostics (Rockville, Maryland) or Simple Plex technology commercialized by Protein Simple (San Jose, CA). Exemplary, array-based methods include, but are not limited to those utilizing Simoa® Planar Array Technology or Simoa® Bead Technology, commercialized by Quanterix (Billerica, MA). Further exemplary array-based methods are set forth in U.S. Pat. Nos. 9,678,068; 9,395,359; 8,415,171; 8,236,574; or 8,222,047, each of which is incorporated herein by reference. Exemplary microfluidic detection methods include those commercialized by Luminex (Austin, Texas) under the trade name xMAP® technology or used on platforms identified as MAGPIX®, LUMINEX® 100/200 or FEXMAP 3D®.

Other detection methods that can also be used, for example at low plex scale, include procedures that employ SOMAmer reagents and SOMAscan assays commercialized by Soma Logic (Boulder, CO). In one configuration, a sample is contacted with aptamers that are capable of binding proteins with specificity for the amino acid sequence of the proteins. The resulting aptamer-protein complexes can be separated from other sample components, for example, by attaching the complexes to beads (or other solid support) that are removed from other sample components. The aptamers can then be isolated and, because the aptamers are nucleic acids, the aptamers can be detected using any of a variety of methods known in the art for detecting nucleic acids, including for example, hybridization to nucleic acid arrays, PCR-based detection, or nucleic acid sequencing. Exemplary methods and compositions are set forth in U.S. Pat. Nos. 7,855,054; 7,964,356; 8,404,830; 8,945,830; 8,975,026; 8,975,388; 9,163,056; 9,938,314; 9,404,919; 9,926,566; 10,221,421; 10,239,908; 10,316,321 10,221,207 or 10,392,621, each of which is incorporated herein by reference.

In some detection assays, a protein can be cyclically modified and the modified products from individual cycles can be detected. In some configurations, a protein can be sequenced by a sequential process in which each cycle includes steps of detecting the protein and removing one or more terminal amino acids from the protein. Optionally, one or more of the steps can include adding a label to the protein, for example, at the amino terminal amino acid or at the carboxy terminal amino acid. In particular configurations, a method of detecting a protein can include steps of (i) exposing a terminal amino acid on the protein; (ii) detecting a change in signal from the protein; and (iii) identifying the type of amino acid that was removed based on the change detected in step (ii). The terminal amino acid can be exposed, for example, by removal of one or more amino acids from the amino terminus or carboxyl terminus of the protein. Steps (i) through (iii) can be repeated to produce a series of signal changes that is indicative of the sequence for the protein.

In a first configuration of a cyclical protein detection method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. No. 9,625,469 or 10,545,153, each of which is incorporated herein by reference. Methods and apparatus under development by Erisyon, Inc. (Austin, TX) may also be useful for detecting proteins.

In a second configuration of a cyclical protein detection method, a terminal amino acid of a protein can be recognized by an affinity agent that is specific for the terminal amino acid or specific for a label moiety that is present on the terminal amino acid. The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in US Pat. App. Pub. No. 2019/0145982 A1; 2020/0348308 A1; or 2020/0348307 A1, each of which is incorporated herein by reference. Methods and apparatus under development by Encodia, Inc. (San Diego, CA) may also be useful for detecting proteins.

Cyclical removal of terminal amino acids from a protein can be carried out using an Edman-type sequencing reaction in which a phenyl isothiocyanate reacts with a N-terminal amino group under mildly alkaline conditions (e.g. about pH 8) to form a cyclical phenylthiocarbamoyl Edman complex derivative. The phenyl isothiocyanate may be substituted or unsubstituted with one or more functional groups, linker groups, or linker groups containing functional groups. An Edman-type sequencing reaction can include variations to reagents and conditions that yield a detectable removal of amino acids from a protein terminus, thereby facilitating determination of the amino acid sequence for a protein or portion thereof. For example, the phenyl group can be replaced with at least one aromatic, heteroaromatic or aliphatic group which may participate in an Edman-type sequencing reaction, non-limiting examples including: pyridine, pyrimidine, pyrazine, pyridazoline, fused aromatic groups such as naphthalene and quinoline), methyl or other alkyl groups or alkyl group derivatives (e.g., alkenyl, alkynyl, cyclo-alkyl). Under certain conditions, for example, acidic conditions of about pH 2, derivatized terminal amino acids may be cleaved, for example, as a thiazolinone derivative. The thiazolinone amino acid derivative under acidic conditions may form a more stable phenylthiohydantoin (PTH) or similar amino acid derivative which can be detected. This procedure can be repeated iteratively for residual protein to identify the subsequent N-terminal amino acid. Many variations of Edman-type degradation have been described and may be used including, for example, a one-step removal of an N-terminal amino acid using alkaline conditions (Chang, J. Y., FEBS LETTS., 1978, 91(1), 63-68). In some cases, Edman-type reactions may be thwarted by N-terminal modifications which may be selectively removed, for example, N-terminal acetylation or formylation (e.g., see Gheorghe M. T., Bergman T. (1995) in Methods in Protein Structure Analysis, Chapter 8: Deacetylation and internal cleavage of Proteins for N-terminal Sequence Analysis. Springer, Boston, MA. https://doi.org/10.1007/978-1-4899-1031-8_8).

Non-limiting examples of functional groups for substituted phenyl isothiocyanate may include ligands (e.g. biotin and biotin analogs) for known receptors, labels such as luminophores, or reactive groups such as click functionalities (e.g. compositions having an azide or acetylene moiety). The functional group may be a DNA, RNA, peptide or small molecule barcode or other tag which may be further processed and/or detected.

The removal of an amino terminal amino acid using Edman-type processes can utilize at least two main steps, the first step includes reacting an isothiocyanate or equivalent with protein N-terminal residues to form a relatively stable Edman complex, for example, a phenylthiocarbamoyl complex. The second step can include removing the derivatized N-terminal amino acid, for example, via heating. The protein, now having been shortened by one amino acid, may be detected, for example, by contacting the protein with a labeled affinity agent that is complementary to the amino terminus and examining the protein for binding to the agent, or by detecting loss of a label that was attached to the removed amino acid.

Edman-type processes can be carried out in a multiplex format to detect, characterize or identify a plurality of proteins. A method of detecting a protein can include steps of (i) exposing a terminal amino acid on a protein at an address of an array; (ii) binding an affinity agent to the terminal amino acid, where the affinity agent includes a nucleic acid tag, and where a primer nucleic acid is present at the address; (iii) extending the primer nucleic acid, thereby producing an extended primer having a copy of the tag; and (iv) detecting the tag of the extended primer. The terminal amino acid can be exposed, for example, by removal of one or more amino acids from the amino terminus or carboxyl terminus of the protein. Steps (i) through (iv) can be repeated to produce a series of tags that is indicative of the sequence for the protein. The method can be applied to a plurality of proteins on the array and in parallel. Whatever the plexity, the extending of the primer can be carried out, for example, by polymerase-based extension of the primer, using the nucleic acid tag as a template. Alternatively, the extending of the primer can be carried out, for example, by ligase- or chemical-based ligation of the primer to a nucleic acid that is hybridized to the nucleic acid tag. The nucleic acid tag can be detected via hybridization to nucleic acid probes (e.g. in an array), amplification-based detections (e.g. PCR-based detection, or rolling circle amplification-based detection) or nuclei acid sequencing (e.g. cyclical reversible terminator methods, nanopore methods, or single molecule, real time detection methods). Exemplary methods that can be used for detecting proteins using nucleic acid tags are set forth in US Pat. App. Pub. No. 2019/0145982 A1; 2020/0348308 A1; or 2020/0348307 A1, each of which is incorporated herein by reference.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

A protein can be detected based on proximity of two or more affinity agents. For example, the two affinity agents can include two components each: a receptor component and a nucleic acid component. When the affinity agents bind in proximity to each other, for example, due to ligands for the respective receptors being on a single protein, or due to the ligands being present on two proteins that associate with each other, the nucleic acids can interact to cause a modification that is indicative of the two ligands being in proximity. Optionally, the modification can be polymerase catalyzed extension of one of the nucleic acids using the other nucleic acid as a template. As another option, one of the nucleic acids can form a template that acts as splint to position other nucleic acids for ligation to an oligonucleotide. Exemplary methods are commercialized by Olink Proteomics AB (Uppsala Sweden) or set forth in U.S. Pat. Nos. 7,306,904; 7,351,528; 8,013,134; 8,268,554 or 9,777,315, each of which is incorporated herein by reference.

### Detection Systems

One or more steps of a method set forth herein can be carried out in a detection system. Accordingly, a detection system can be configured to execute one or more steps of a method set forth herein. For example, a detection system can be configured to execute one or more steps of a decoding method set forth herein. A decoding method set forth herein can be configured to improve the accuracy of the detection system. For example, the detection system can provide an initial identity or characterization for one or more extant proteins and a decoding method set forth herein can be used to output a subsequent identity or characterization that is more accurate or otherwise improved compared to the initial identity or characterization.

The present disclosure provides a detection system that include (a) a detector configured to acquire signals from a plurality of binding reactions occurring between a plurality of different affinity reagents and a plurality of extant proteins in a sample; (b) a database including information characterizing or identifying a plurality of candidate proteins; (c) a computer processor configured to: (i) communicate with the database, (ii) process the signals to produce a plurality of binding profiles, wherein each of the binding profiles includes a plurality of binding outcomes for binding of an extant protein of (a) to the plurality of different affinity reagents, wherein individual binding outcomes of the plurality of binding outcomes include a measure of binding between an extant protein of (a) and a different affinity reagent of the plurality of different affinity reagents, each of the binding profiles including positive binding outcomes and negative binding outcomes, (iii) process the binding profiles to determine a probability for each of the affinity reagents binding to each of the candidate proteins in the database according to a binding model for each of the affinity reagents; and (iv) outputting an identification of selected candidate proteins, the selected candidate proteins being candidate proteins in the database having a probability for binding each of the affinity reagents that is most compatible with the plurality of binding outcomes for the extant proteins.

A method for identifying an extant protein can be carried out in a detection system. The method can include (a) acquiring signals from a plurality of binding reactions carried out in a detection system, wherein the binding reactions include contacting a plurality of different affinity reagents with a plurality of extant proteins in a sample; (b) processing the signals in the detection system to produce a plurality of binding profiles, wherein each of the binding profiles includes a plurality of binding outcomes for binding of an extant protein of step (a) to the plurality of different binding reagents, wherein individual binding outcomes of the plurality of binding outcomes include a measure of binding between an extant protein of step (a) and a different affinity reagent of the plurality of different binding reagents, each of the binding profiles including positive binding outcomes and negative binding outcomes; (c) providing as inputs to the detection system a database including information characterizing or identifying a plurality of candidate proteins; (d) providing as inputs to the detection system a binding model for each of the different affinity reagents; (e) processing the plurality of binding profiles in the detection system to determine a probability for each of the binding reagents binding to each of the candidate proteins in the database according to the binding model; and (f) outputting from the detection system an identification of selected candidate proteins, the selected candidate proteins being candidate proteins in the database having a probability for binding each of the binding reagents that is most compatible with the plurality of binding outcomes for the extant proteins.

The present disclosure further provides a system for identifying proteins, including (a) a detector configured to acquire signals from a plurality of binding reactions occurring between a plurality of different affinity reagents and a plurality of extant proteins in a sample; (b) a database including: (i) a plurality of candidate outcome profiles, individual candidate outcome profiles of the plurality of candidate outcome profiles each including a plurality of statistical measures for a candidate protein, and (ii) a plurality of pseudo outcome profiles, individual pseudo outcome profiles of the plurality of pseudo outcome profiles each including a plurality of statistical measures that is known to not occur for any of the candidate proteins; (c) a computer processor configured to: (i) acquire a plurality of empirical binding profiles from the signals, wherein each of the empirical binding profiles includes a plurality of binding outcomes for binding of an extant protein of the sample to the plurality of different affinity reagents; (ii) identify extant proteins of the plurality of different extant proteins based on the empirical binding profiles of the extant proteins and the plurality of candidate outcome profiles; and (iii) compute a false discovery statistic for the extant proteins based on the empirical binding profiles of the extant proteins and the plurality of pseudo outcome profiles.

A detection system of the present disclosure can include: (a) a detector configured to detect measurement outcomes for reactions of a plurality of assay reagents with an array of addresses, each of the addresses having an extant protein of a plurality of different extant proteins; (b) a database including a plurality of candidate proteins; and (c) a computer processor configured to: (i) add a measurement outcome of (a) to an outcome profile of an individual address of the array; (ii) determine a collection of probabilities for each of the candidate proteins in the database producing the outcome profile; (iii) determine information entropy for the collection of probabilities; and (iv) repeat (i) through (iii). Optionally, the computer processor is configured to output an identity for the extant protein at the individual address. Alternatively or additionally, the computer processor can be configured to output a measure of the information entropy.

In some configurations, a detection system can include: (a) a detector configured to detect binding outcomes for binding of a plurality of affinity reagents to an array of addresses, each of the addresses having an extant protein of a plurality of different extant proteins; (b) a database including a plurality of candidate proteins; (c) a binding model for each of the different affinity reagents; and (d) a computer processor configured to: (i) add a binding outcome of (a) to a binding profile of an individual address of the array; (ii) evaluate the binding model to determine a collection of probabilities for each of the candidate proteins in the database producing the binding profile; (iii) determine information entropy for the collection of probabilities; and (iv) repeat (i) through (iii). Optionally, the computer processor is configured to output an identity for the extant protein at the individual address, wherein the extant protein is identified as a candidate protein in the database. Alternatively or additionally, the computer processor can be configured to output a measure of the information entropy.

A detection system can include a detector, such as those known in the art for detecting a label or analyte set forth herein. A detector can be configured to collect signals (e.g. optical signals) from an array or other vessel containing extant proteins or other analytes. A camera such as a complementary metal-oxide-semiconductor (CMOS) or charge-coupled device (CCD) camera can be particularly useful, for example, to detect optical labels such as luminophores. The detection system can further include an excitation source configured to excite extant proteins, affinity reagents or other analytes, for example, in an array or other vessel. A detection system can include a scanning mechanism configured to effect relative movement between a detector and an array or other vessel containing extant proteins. Optionally, the scanning mechanism can be configured for time-delayed integration. Detectors that are capable of resolving proteins on an array surface including, for example, at single-molecule resolution can be particularly useful. Detectors used in DNA sequencing systems can be modified for use in a detection system or other apparatus set forth herein. Exemplary detectors are described, for example, in U.S. Pat. Nos. 7,057,026; 7,329,492; 7,211,414; 7,315,019 or 7,405,281, or US Pat. App. Pub. No. 2008/0108082 A1, each of which is incorporated herein by reference.

A detection system can further include fluidics apparatus configured to contact reaction components for a reaction or other step of a method set forth herein. In particular embodiments, reactions occur on arrays. Any of a variety of arrays can be present in the system, such as an array set forth herein. Proteins that are to be detected, for example those attached to an array, can be housed in any of a variety of reaction vessels. A particularly useful reaction vessel is a flow cell. A flow cell or other vessel can be present in a system in a permanent manner or in a removable manner, for example, being removable by hand or without the use of an auxiliary tool. A flow cell or other vessel that is present in a system can have a detection window through which a detector observes one or more proteins (e.g. an array of proteins) or other analytes on the array. For example, an optically transparent window can be used in conjunction with an optical detector such as a fluorimeter or luminescence detector.

A fluidic apparatus can include one or more reservoirs which are fluidically connected to an inlet of a flow cell or other vessel. The reservoirs can include reagents for use in a method set forth herein. The system can further include a pump, pressure supply or other fluid displacement apparatus for driving reagents from reservoirs to the vessel. The system can include a waste reservoir that is fluidically connected to an egress of a vessel to remove spent reagents. Taking as an example an embodiment where the vessel is a flow cell, reagents can be delivered to the flow cell through a flow cell ingress and then the reagents can flow through the flow cell and out the flow cell egress to a waste reservoir. Accordingly, the flow cell can be in fluidic communication with one or more reservoirs of the system. A fluidic system can include at least one manifold and/or at least one valve for directing reagents from reservoirs to a vessel where detection occurs. Exemplary fluidic apparatus that can be used in a system of the present disclosure include those configured for cyclic delivery of reagents, such as those deployed in nucleic acid sequencing reactions. Exemplary fluidic apparatus are set forth in US Pat. App. Pub. Nos. 2009/0026082 A1; 2009/0127589 A1; 2010/0111768 A1; 2010/0137143 A1; or 2010/0282617 A1; or U.S. Pat. Nos. 7,329,860; 8,951,781 or 9,193,996, each of which is incorporated herein by reference.

### Computer Systems

The present disclosure provides computer systems (e.g. computer control systems) that are programmed to implement methods, processes or functions set forth herein. Optionally, a computer system set forth herein can be a component of a detection system. Optionally, a computer system can be programmed or otherwise configured to: (a) receive an input set forth herein such as a binding profile, a pseudo outcome profile, a database comprising information characterizing or identifying a plurality of candidate proteins, a binding model and/or non-specific binding rates for affinity reagents, (b) determine probabilities for affinity reagents binding to candidate proteins, for example, based on a binding model, (c) identify extant proteins as selected candidate proteins, (d) generate pseudo outcome profiles, and/or (e) determine a false discovery statistic.

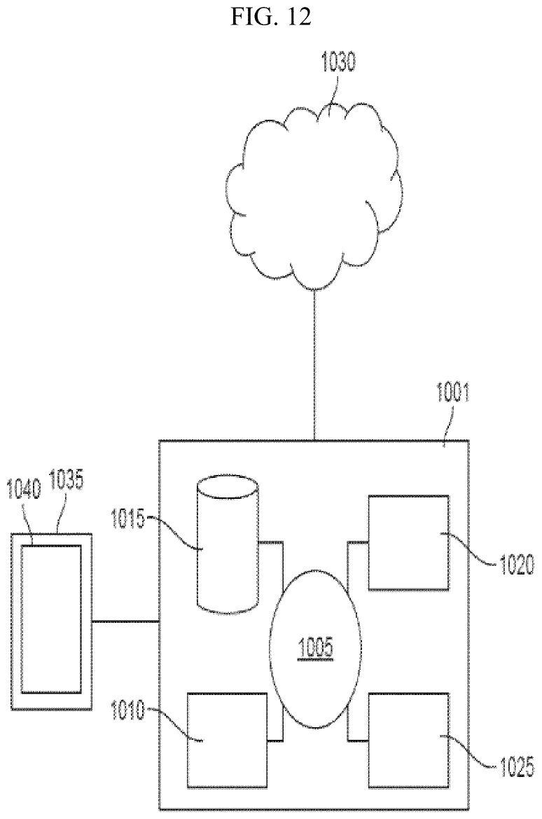

FIG. 12 shows an exemplary computer system 1001. The computer system 1001 can be an electronic device of a detection system, the electronic device being integral to the detection system or remotely located with respect to the detection system. For example, the electronic device can be a mobile electronic device. The computer system 1001 includes a computer processing unit (CPU, also “processor” and “computer processor” herein) 1005, which can be a single core or multi core processor, or a plurality of processors for parallel processing. The computer system 1001 also includes memory or memory location 1010 (e.g., random-access memory, read-only memory, flash memory), electronic storage unit 1015 (e.g., hard disk), communication interface 1020 (e.g., network adapter) for communicating with one or more other systems, and peripheral devices 1025, such as cache, other memory, data storage and/or electronic display adapters. The memory 1010, storage unit 1015, interface 1020 and peripheral devices 1025 are in communication with the CPU 1005 through a communication bus (solid lines), such as a motherboard. The storage unit 1015 can be a data storage unit (or data repository) for storing data. The computer system 1001 can be operatively coupled to a computer network (“network”) 1030 with the aid of the communication interface 1020. The network 1030 can be the Internet, an internet and/or extranet, or an intranet and/or extranet that is in communication with the Internet. The network 1030 in some cases is a telecommunication and/or data network. The network 1030 can include one or more computer servers, which can enable distributed computing, such as cloud computing. For example, one or more computer servers may enable cloud computing over the network 1030 (“the cloud”) to perform various aspects of analysis, calculation, and generation of the present disclosure, such as, for example, receiving information of empirical measurements of extant proteins in a sample; processing information of empirical measurements against a database comprising a plurality of protein sequences corresponding to candidate proteins, for example, using a binding model or function set forth herein; generating probabilities of a candidate protein generating empirical measurements, and/or generating probabilities that extant proteins are correctly identified in the sample. Such cloud computing may be provided by cloud computing platforms such as, for example, Amazon Web Services (AWS), Microsoft Azure, Google Cloud Platform, and IBM cloud. The network 1030, in some cases with the aid of the computer system 1001, can implement a peer-to-peer network, which may enable devices coupled to the computer system 1001 to behave as a client or a server.

The CPU 1005 can execute a sequence of machine-readable instructions, which can be embodied in a program or software. The instructions may be stored in a memory location, such as the memory 1010. The instructions can be directed to the CPU 1005, which can subsequently program or otherwise configure the CPU 1005 to implement methods of the present disclosure. Examples of operations performed by the CPU 1005 can include fetch, decode, execute, and writeback.

The CPU 1005 can be part of a circuit, such as an integrated circuit. One or more other components of the system 1001 can be included in the circuit. In some cases, the circuit is an application specific integrated circuit (ASIC).

The storage unit 1015 can store files, such as drivers, libraries and saved programs. The storage unit 1015 can store user data, e.g., user preferences and user programs. The computer system 1001 in some cases can include one or more additional data storage units that are external to the computer system 1001, such as located on a remote server that is in communication with the computer system 1001 through an intranet or the Internet.

The computer system 1001 can communicate with one or more remote computer systems through the network 1030. For instance, the computer system 1001 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1001 via the network 1030.

Methods as described herein can be implemented by way of machine (e.g., computer processor) executable code stored on an electronic storage location of the computer system 1001, such as, for example, on the memory 1010 or electronic storage unit 1015. The machine executable or machine readable code can be provided in the form of software. During use, the code can be executed by the processor 1005. In some cases, the code can be retrieved from the storage unit 1015 and stored on the memory 1010 for ready access by the processor 1005. In some situations, the electronic storage unit 1015 can be precluded, and machine-executable instructions are stored on memory 1010.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1001, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1001 can include or be in communication with an electronic display 1035 that comprises a user interface (UI) 1040 for providing, for example, user selection of processes, binding measurement data, candidate proteins, and databases. Examples of UIs include, without limitation, a graphical user interface (GUI) and web-based user interface.

Methods and systems of the present disclosure can be implemented by way of one or more processes. A process can be implemented by way of software upon execution by the central processing unit 1005. The process can, for example, receive information of empirical measurements of extant proteins in a sample, compare information of empirical measurements against a database comprising a plurality of protein sequences corresponding to candidate proteins, generate probabilities of a candidate protein generating the observed measurement outcome set, and/or generate probabilities that candidate proteins are correctly identified in the sample.

The present disclosure provides a non-transitory information-recording medium that has, encoded thereon, instructions for the execution of one or more steps of the methods set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Example I

### Single-Molecule Protein Identification Using Multi-Affinity Protein Affinity Reagents

This example describes a foundation for high-throughput single-molecule protein identification. The approach uses multi-affinity reagents that bind short, linear epitopes with low specificity and a decoding process that accommodates stochasticity expected for single-molecule binding. In simulations, the approach achieved high proteome coverage in a wide range of organisms and was robust to potential experimental confounders. Simulating a human blood plasma proteome experiment, the approach supported a dynamic range of detection spanning at least eight orders of magnitude. The results indicated that, if executed experimentally, the approach could quantitatively decode over 90% of the human proteome in a single experiment, potentially revolutionizing proteomics research.

### Results and Discussion

As a preliminary matter, the present example sets forth methods that can be used to identify and distinguish proteins based on their primary structure (i.e. amino acid sequence). In this context, reference to proteins differing, whether implied or explicit, pertains to differences in their primary structure. Notwithstanding the foregoing, the methods exemplified herein can be useful, in some cases by adaptation that will be apparent to those skilled in the art, to identifying proteins based on differences such as presence, number, type or location of post translational modifications.

FIG. 1A shows an experimental setup for detecting a plurality of proteins at single molecule resolution. Proteins are extracted from a sample and each protein is conjugated in a denatured state to a structured nucleic acid particle (SNAP) followed by deposition of the protein-conjugated SNAP on a solid support having 10¹⁰ addresses. No more than one protein-conjugated SNAP binds per address, creating a hyper-dense single molecule array with each address having a protein that is optically resolvable from neighboring addresses. A series of affinity reagents (e.g. antibodies, aptamers, or small proteins), tagged with fluorophores, is contacted with the array. One affinity reagent is used per cycle of the series, presence or absence of binding is detected at each address and the affinity reagent is washed off the array before the next one is added via the next cycle. Integrated fluidics and imaging on instrument allow high resolution multi-cycle imaging of the addresses in the presence of the affinity reagents. Therefore, binding of affinity reagents to proteins produces a series of bind/no-bind outcomes for each protein, which can be used to infer the identity of the protein. Since there is only one protein per address, direct counting of the addresses can be used to quantify each protein identified in the sample.

Identifying the many different proteins in a human proteome, or other complex proteome, would require a prohibitively large number of highly specific affinity reagents. The present methods overcome this by using affinity reagents that bind short, linear epitopes (e.g., trimers) with moderate specificity, so that each affinity reagent binds many different proteins. While binding of a single affinity reagent is insufficient to identify any particular protein with these promiscuous affinity reagents, a series of affinity reagents can decode many different proteins. The detection of each new affinity reagent bound at each address across a growing number of cycles gradually narrows down the list of possible protein identities at each address (FIG. 1B).

In a typical single-molecule binding reaction format, binding is stochastic, as an affinity reagent will not always be observed to bind a protein containing its epitope (see Chang, et al., J Immunol Methods 378, 102-115 (2012), which is incorporated herein by reference). Furthermore, each affinity reagent may be observed to bind to off-target epitopes. Therefore, repeating the same series of single-molecule binding reactions multiple times will typically result in observation of multiple different binding patterns (FIG. 1C).

In view of this stochasticity, a binding model was devised whereby each affinity reagent binds with a primary probability to a protein containing one copy of its target epitope and with an equal or lower probability to a protein containing one copy of an off-target epitope. The rather low probability of 0.5 was initially selected for on-target binding to its primary epitope and 0.5 probability to binding to an off-target epitope because there are many factors that could prevent binding of an affinity reagent to its epitope, for example, residual or transient protein structure due to partial denaturation, presence of post-translational modifications, binding stochasticity or the like. To determine the affinity reagent selectivity that provides high coverage of the human proteome with a manageable number of different affinity reagents, affinity reagents with various target epitope lengths (dimer, trimer, or tetramer) and varying numbers of off-target epitopes were evaluated. As shown in FIG. 1D, the analysis showed that 100 affinity reagents would facilitate unique identification of 90% of the human proteome if each affinity reagent bound to a single trimer and 9 additional primary off-target trimers. In this scenario, each affinity reagent would bind about 23.7% of the proteins (N.B. the percentage being based on the number of unique protein sequences independent of variability in expression level for each protein) in the human proteome and about 24 binding events would be sufficient to identify a given protein on average (Table 1). Targeting tetramer epitopes would reduce the number of binding events but increase the number of affinity reagents sufficient to achieve similar coverage. Targeting dimer epitopes would allow for a similar number of affinity reagents, but it could be challenging to generate affinity reagents that recognize dimers independent of variability in the sequence surrounding the dimer. Therefore ‘timer with 10 epitopes’ affinity reagent selectivity model was used for the present analyses.

TABLE 1
Affinity Reagent Characteristics

| | | | Number of | Number of | % Landing | % Landing |
|---|---|---|---|---|---|---|
| | Number | Number | Affinity | Affinity | Pads Lit Per | Pads Lit Per |
| | of Epitopes | of Cycles | Reagents Bound | Reagents Bound | Affinity | Affinity |
| Epitope | per Affinity | for 90% | Per Protein | Per Protein | Reagent | Reagent |
| Type | Reagent | Coverage | (mean) | (std dev) | (mean) | (std dev) |
| Dimer | 1 | 110 | 40.30728935 | 19.55502431 | 36.64299032 | 14.84719629 |
| Dimer | 2 | >2000 | 1102.618384 | 400.209814 | 55.1309192 | 15.78071197 |
| Trimer | 1 | 410 | 12.53679269 | 11.32908544 | 3.057754314 | 2.335865074 |
| Trimer | 2 | 250 | 14.44343958 | 12.2351679 | 5.777375834 | 3.636218605 |
| Trimer | 5 | 130 | 17.64635532 | 12.8720439 | 13.57411948 | 6.87105185 |
| Trimer | 10 | 100 | 23.70956264 | 14.70880367 | 23.70956264 | 10.00887062 |
| Trimer | 20 | 120 | 44.13743514 | 21.73416995 | 36.78119595 | 14.22655523 |
| Trimer | 25 | 150 | 62.95384235 | 28.56932135 | 41.96922823 | 14.86283342 |
| Trimer | 30 | 220 | 100.8525822 | 42.68880344 | 45.8420828 | 15.54123936 |
| Trimer | 40 | 690 | 361.7437608 | 136.1278615 | 52.426632 | 16.54646207 |
| Tetramer | 1 | >2000 | 3.552409192 | 3.996546384 | 0.17762046 | 0.215077644 |
| Tetramer | 2 | >2000 | 6.561551767 | 6.901374069 | 0.328077588 | 0.344402837 |
| Tetramer | 5 | 1350 | 10.71208302 | 10.60025175 | 0.793487631 | 0.730490262 |
| Tetramer | 10 | 760 | 11.43864591 | 10.92199034 | 1.505084988 | 1.22358617 |
| Tetramer | 20 | 430 | 12.63602669 | 11.43641187 | 2.938610857 | 2.210826628 |
| Tetramer | 25 | 370 | 13.20800593 | 11.69248829 | 3.569731333 | 2.62810432 |
| Tetramer | 30 | 320 | 13.50155671 | 11.71124183 | 4.219236471 | 3.073673392 |

It is also possible to use affinity reagents that are more specific, for example, binding to a single epitope or even a single protein. In some cases, multiple different affinity reagents can be combined to create a pool of affinity reagents that binds with apparent promiscuity. For example, a pool of 3 different affinity reagents that are indistinguishably detected from each other in a binding step would appear to promiscuously bind proteins targeted by the pool. By way of more specific example, a pool of 3 different affinity reagents may apparently bind at least 3 different proteins, a pool of 5 different affinity reagents may apparently bind at least 5 different proteins, a pool of 10 different affinity reagents could apparently bind at least 10 different proteins, etc.

In addition to having primary binding epitopes, affinity reagents are likely to bind other off-target epitopes, albeit with lower probability. A “biosimilar” affinity reagent model (see Methods section below) was used, whereby each affinity reagent had a “tail” of up to 20 additional secondary off-target epitopes, with binding probabilities proportional to the similarity of the off-target epitope to the target epitope. Using this model with target epitopes selected randomly from targets present in the human proteome, the decoding process was able to uniquely identify about 98% of proteins in the human proteome (modeling a sample with one copy of each protein) with 300 cycles (FIG. 1E). Performance with less than 200 affinity reagents improved when using a greedy-selection process (see Methods section below) to determine the optimal set of 300 trimer epitopes achieving high human proteome coverage with as few affinity reagent cycles as possible (FIG. 1E). This optimal set of epitopes was used for subsequent analyses.

To test whether the decoding strategy can be applied to proteomes from species other than humans, the same parameters were used with the same set of optimized affinity reagents to simulate analysis of proteomes from mouse, S. cerevisiae, and E. coli (FIG. 1F). Surprisingly, there was little difference between the species, indicating that while smaller proteomes are slightly easier to decode, the primary driver of decoding performance is protein sequence diversity. Therefore, despite the stochastic nature of single molecule binding, the decoding strategy has the potential to decode more than 90% of the proteome for a wide range of organisms.

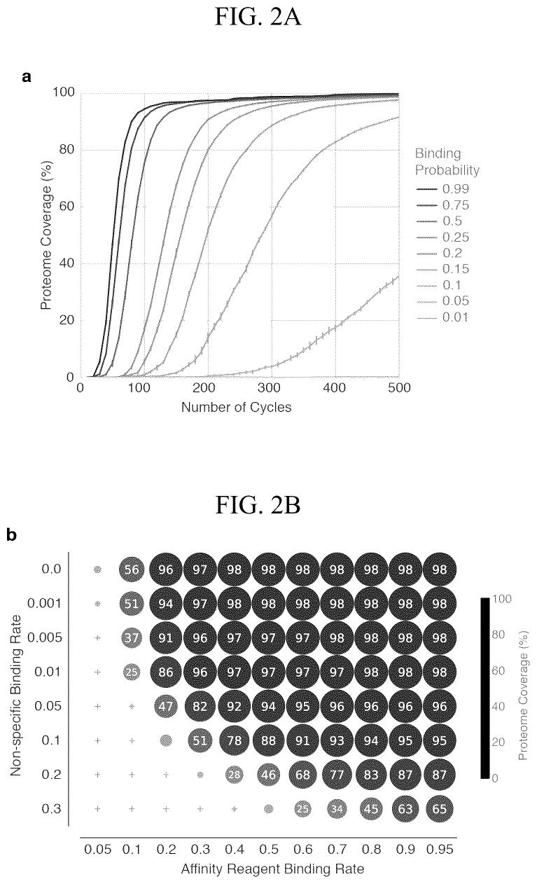

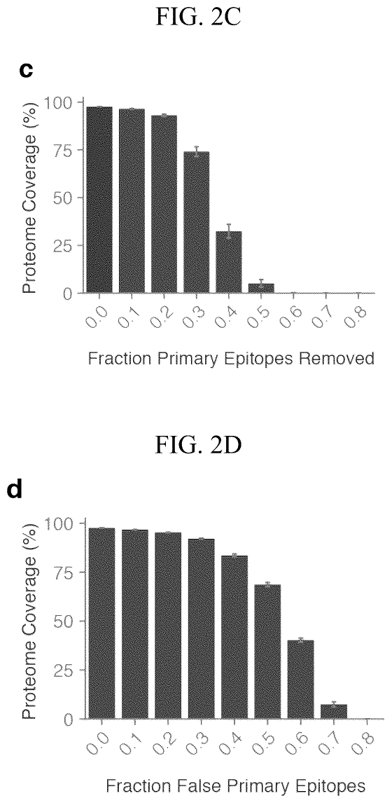

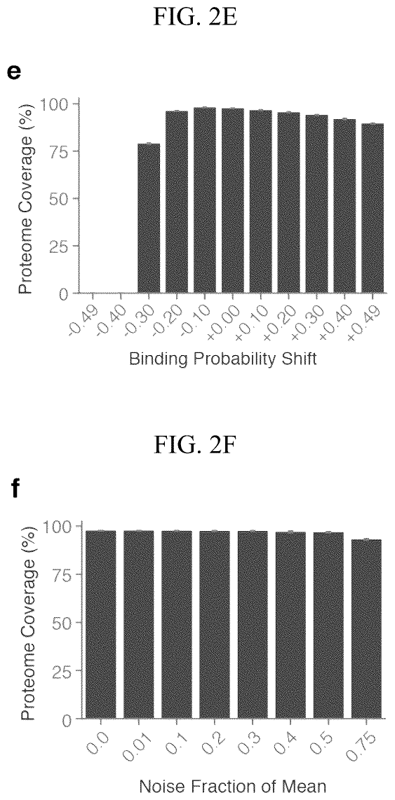

Potential experimental confounders were evaluated. A first scenario, in which the probability of affinity reagent to epitope binding is even lower than 0.5, for example due to poor binding affinity or kinetics, was considered. Even with a probability of 0.1, the decoding method achieved over 85% proteome coverage using 300 cycles (i.e. 300 different affinity reagents), although this dropped to about 55% when the binding probability was 0.05 (FIG. 2A). Options for increasing coverage include, for example, using more affinity reagents, multiplexing several affinity reagents in a single run (for example, using different fluorescent labels for each probe in a multiplexed set); running affinity reagents in replicate cycles to improve the chances of observing binding; increasing concentration of affinity reagents; increasing duration of the binding reaction; or attaching multiple copies of an affinity reagent to a scaffold such as a fluorescent particle or structured nucleic acid particle. Accordingly, the decoding method may be viable using affinity reagents across a range of binding probabilities, some of which are relatively low.

The effect of non-specific binding of an affinity reagent to the surface of an array at a location close enough to a protein address to create a false binding signal was evaluated. As demonstrated by FIG. 2B, assuming a binding probability of 0.5, a non-specific binding rate of 0.05 or lower provided about 90% detection sensitivity. For subsequent analyses, a non-specific binding rate of 0.001 was assumed. If the rate proves to be higher experimentally, binding conditions (e.g. ionic strength, temperature, polarity, pH, osmolarity, concentration of affinity reagent or surface tension) can be adjusted to reduce non-specific binding. The same or different conditions can be used for each affinity reagent.

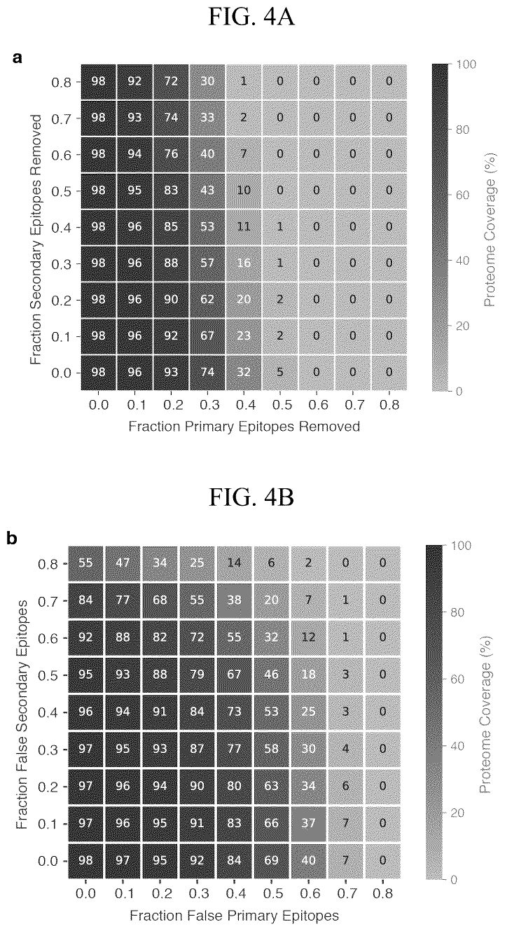

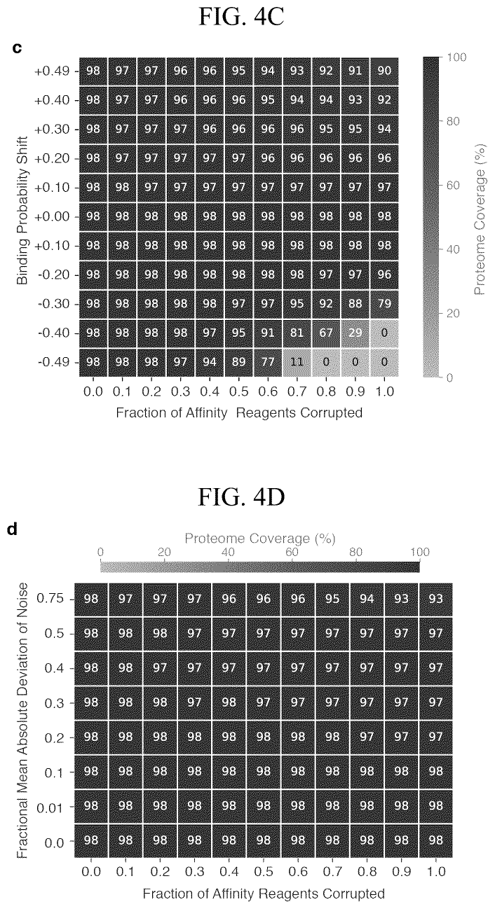

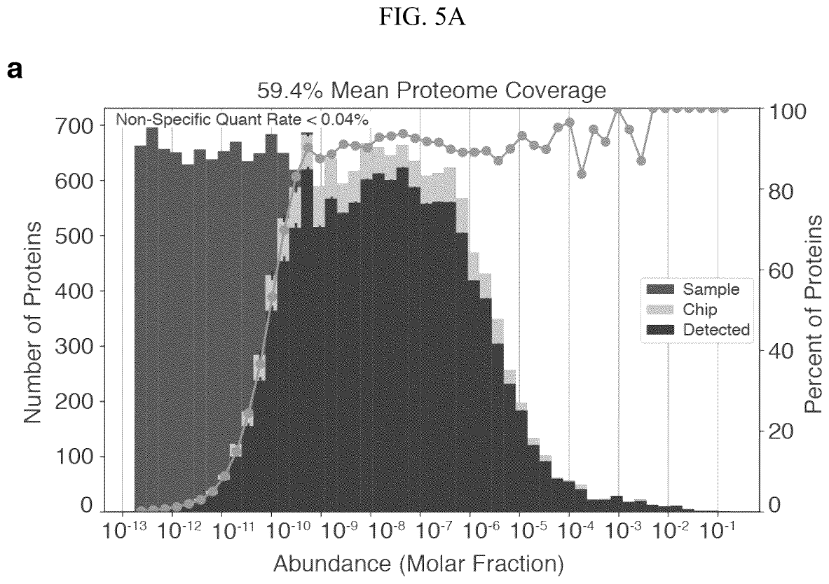

The impact of affinity reagent characterization (e.g. identification of target epitopes and off-target epitopes, and the respective binding probabilities) was also evaluated. Such characterization can be performed in a straightforward manner using traditional epitope mapping approaches (Beyer, et al., Science 318, 1888 (2007), which is incorporated herein by reference). Trimer epitopes may be “missed” during affinity reagent characterization, for example, if each affinity reagent binds an additional number of epitopes that the inference process does not know about (FIG. 2C, FIG. 4A). However, the impact was small, so long as high probability (0.5) binding epitopes were not consistently missed. Proteome coverage remained above 92% if up to 20% of these epitopes were missed. Trimer epitopes may also be falsely identified as targets during affinity reagent characterization (FIG. 2D, FIG. 4B). The decoding method appeared to be robust to this type of error, as it achieved nearly 70% coverage even if half of all primary epitopes were incorrect. Given that the decoding method appeared to be more robust to having false positive epitopes than ‘missing’ epitopes in the affinity reagent model, the techniques used to characterize affinity reagents can be tuned more towards sensitivity rather than specificity to achieve improved results. Evaluation of the impact of consistent over- or under-estimation of affinity reagent to epitope binding probabilities indicated that the impact of such errors was small with the exception of large (>−0.2) underestimation of binding probability (FIG. 2E, FIG. 4C). The decoding method appeared to be highly robust to noisy affinity reagent characterization, indicating that affinity reagent characterization need not be perfect, and that the method will tolerate variability in affinity reagent binding characteristics that may arise from other potential experimental confounders such as temperature (FIG. 2F, FIG. 4D). In summary, the decoding method appeared to be robust to errors in the affinity reagent characterization.

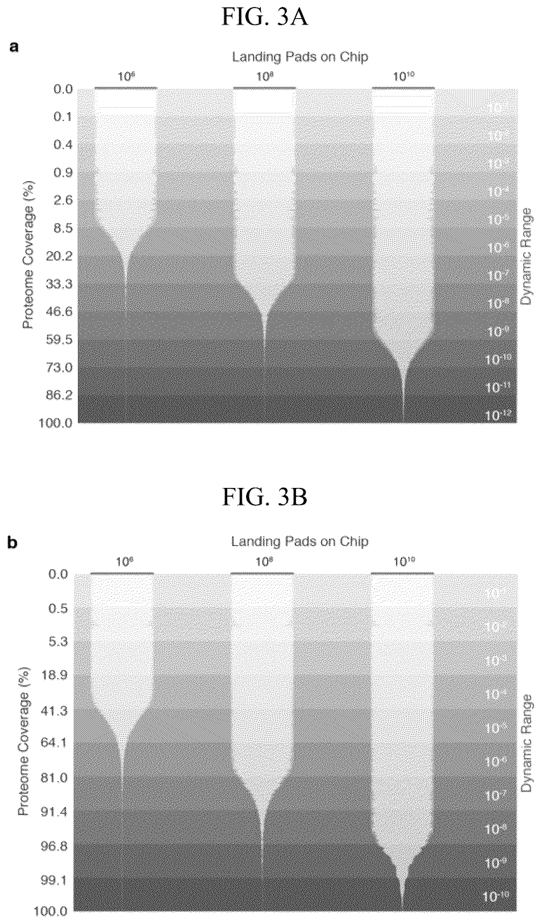

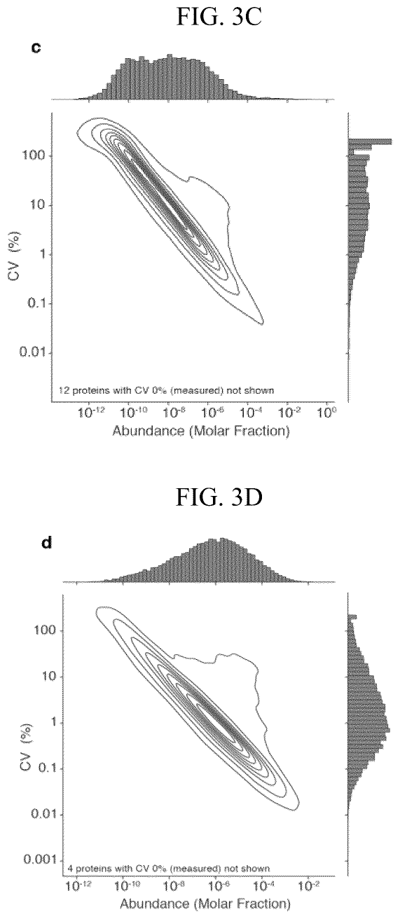

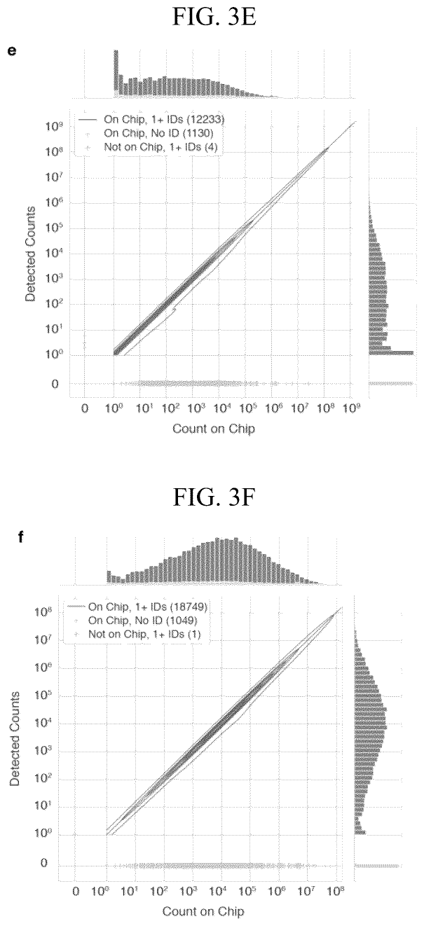

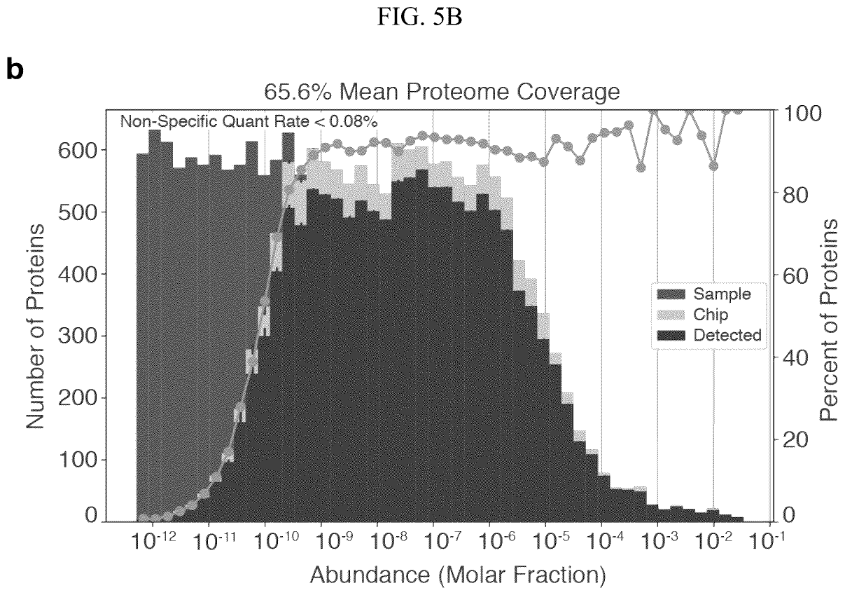

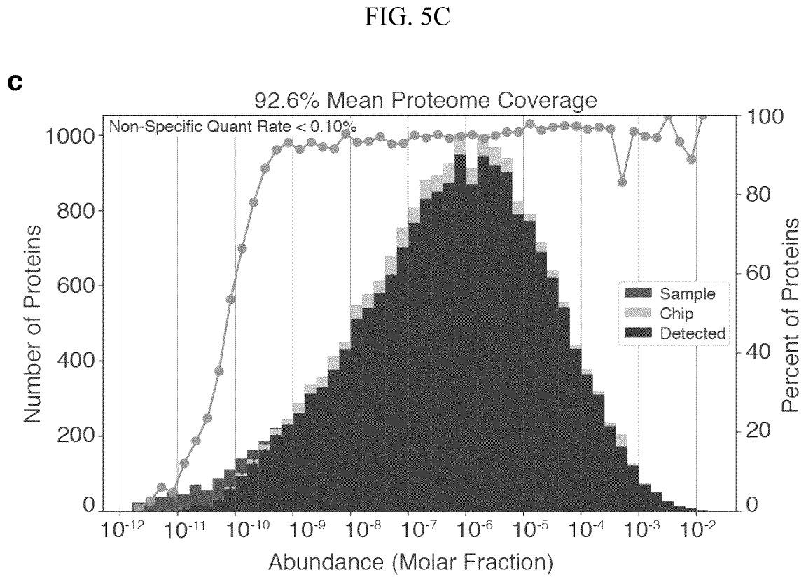

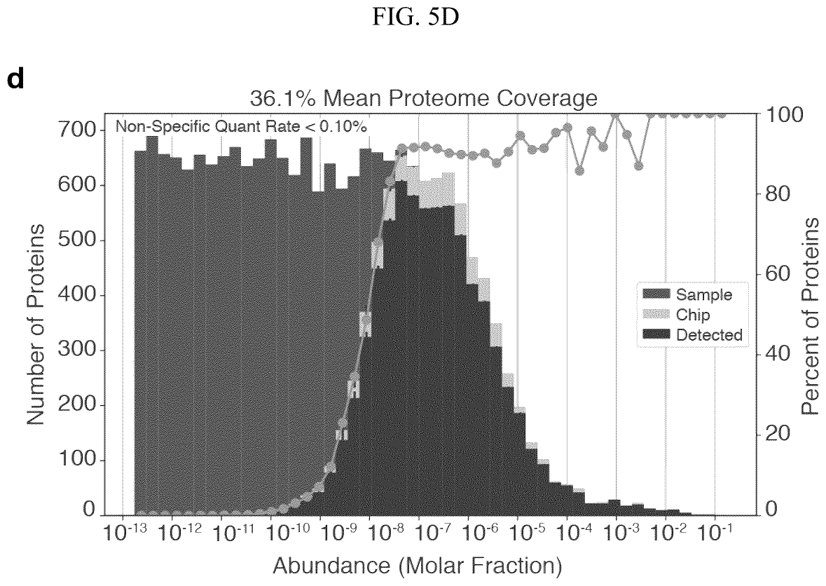

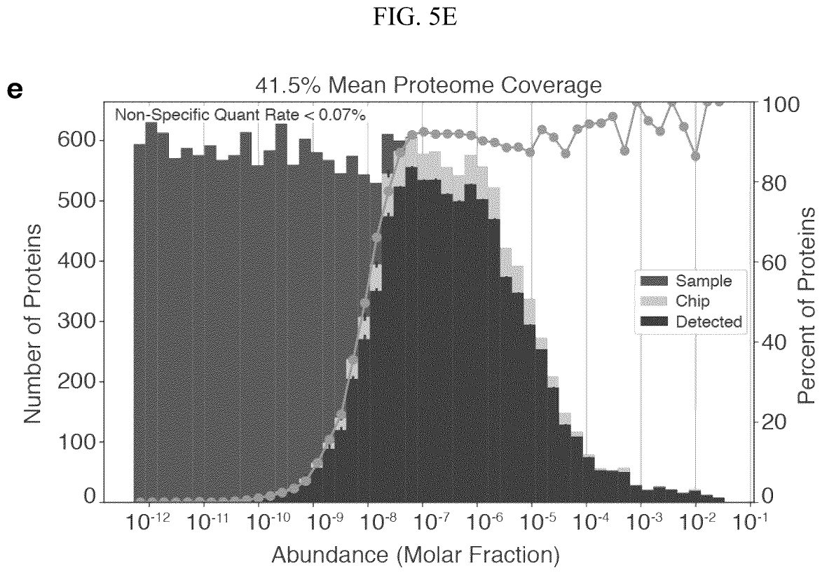

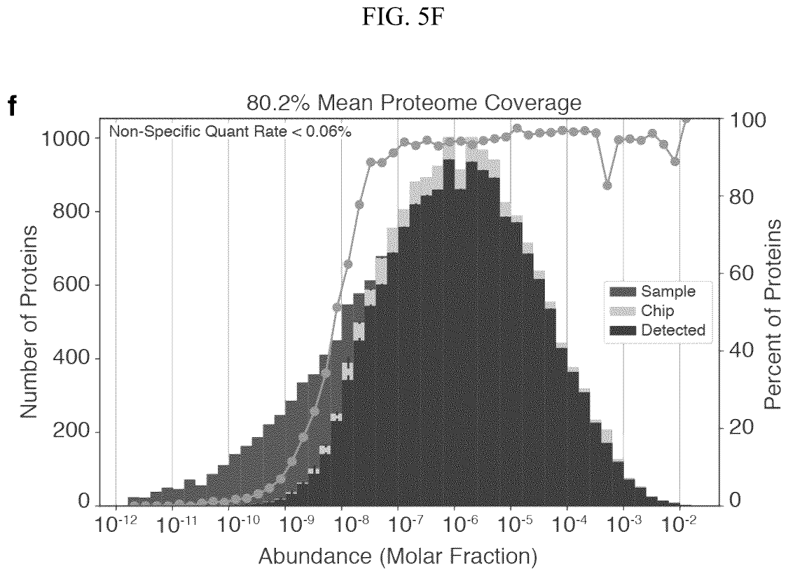

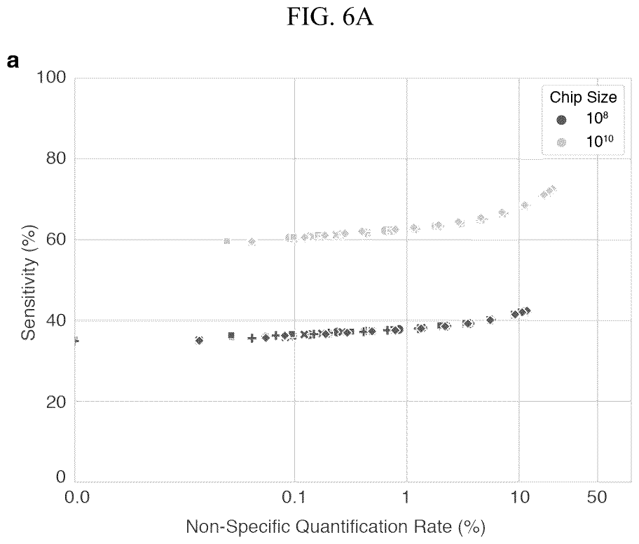

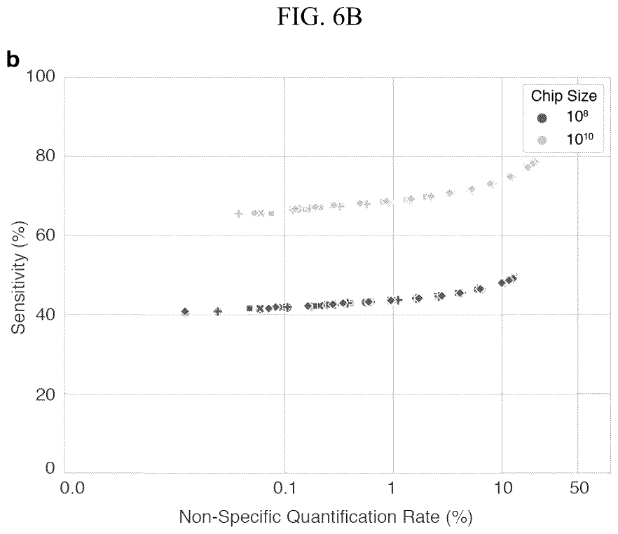

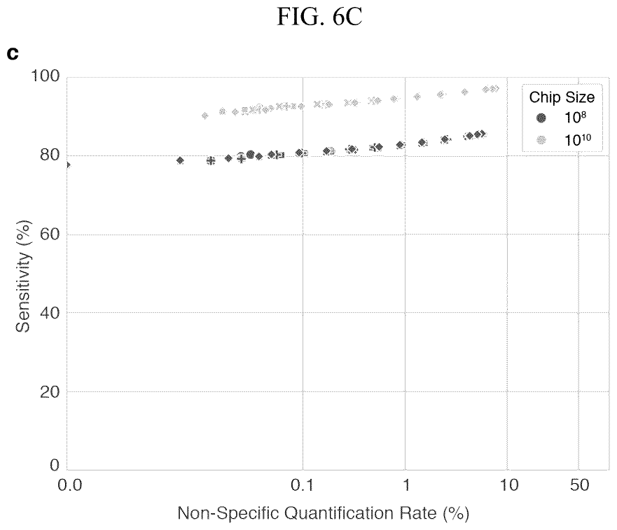

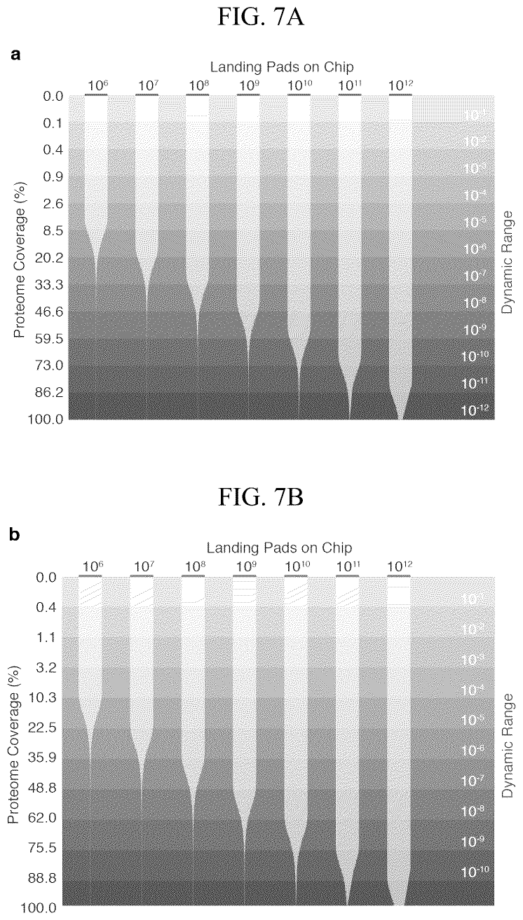

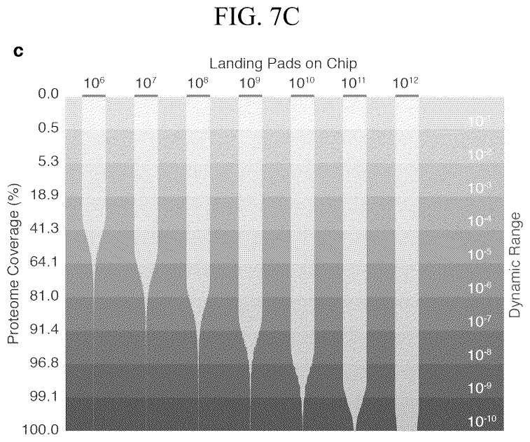

Blood plasma is a good example of one of the major challenges to proteomics, as plasma protein concentrations can vary by more than 12 orders of magnitude and typical mass spectrometry-based approaches typically only identify 8% of the proteome (see Anderson & Anderson, Mol Cell Proteomics 1, 845-867 (2002), which is incorporated herein by reference). To evaluate the theoretical performance of the protein decoding strategy, a simulation was run for assaying an un-depleted blood plasma sample with 300 affinity reagents on an array with 10⁶, 10⁸ and 10¹⁰ addresses. The simulation modeled running the same sample across five technical replicates. Some random noise in affinity reagent to trimer binding probability simulated variability in affinity reagent binding across replicates. On average, simulations executing the decoding process with a 10¹⁰ address array demonstrated a detection dynamic range spanning >11.5 orders of magnitude ranging from the most abundant to the least abundant protein detected (FIG. 3A, FIGS. 5A-5F). The decoding method was able to quantify 59.4% of the 20,235 proteins in the modeled plasma sample. Almost all proteins were quantified with high specificity (FIGS. 6A-6C). More than 99.6% of the measured proteins had quantitative specificity >90% (i.e., >90% of identifications of the protein were true positives). Proteins within the top 9 orders of magnitude dynamic range were detected with 90% consistency. Bias in identifiability that correlated with protein concentration was not observed. Overall, 90% of proteins deposited on the array were detected, indicating that the ability to deposit low concentration proteins on the array, rather than the ability to decode proteins, is the primary limiter of dynamic range. Modeling suggests that increasing the number of addresses to 10¹¹ or 10¹² would increase identification of proteins deposited on the array from 66% to 79% and 92%, respectively (FIGS. 7A-7C)

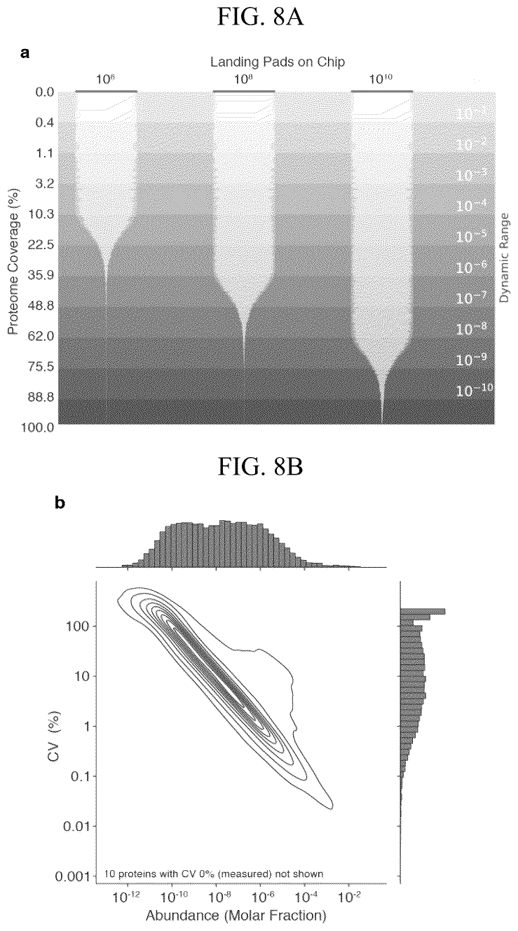

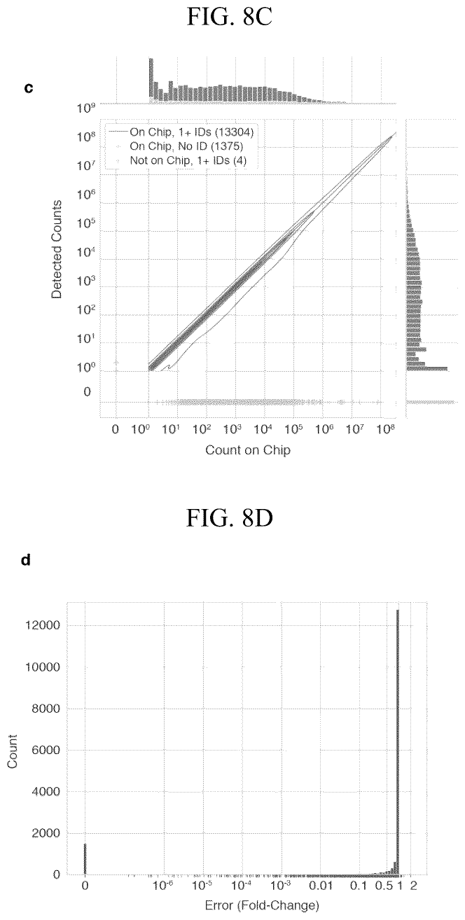

Experimentally, the dynamic range could be compressed by depleting the most abundant proteins in a plasma sample, for example, using an affinity column. A plasma sample modeled with 99% depletion of the top 20 proteins had 65.7% proteome coverage on average (FIGS. 8A-8D). Coverage was substantially higher (92.6%) when modeling a HeLa cell-line sample, which has a lower dynamic range (detection spanned 9.5 orders of magnitude) (FIG. 3B).

In all samples, some proteins with relatively high abundance were not detected because detectability is not just a factor of abundance but also sequence similarity. If the sequence of a protein is very similar to another protein in the database, it can be difficult for the decoding process to generate confident identifications for these proteins. More selective affinity reagents can be used to detect these more difficult targets.

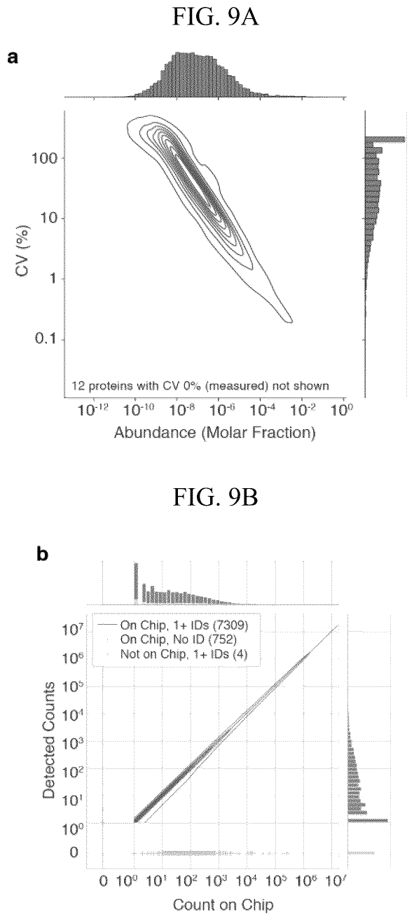

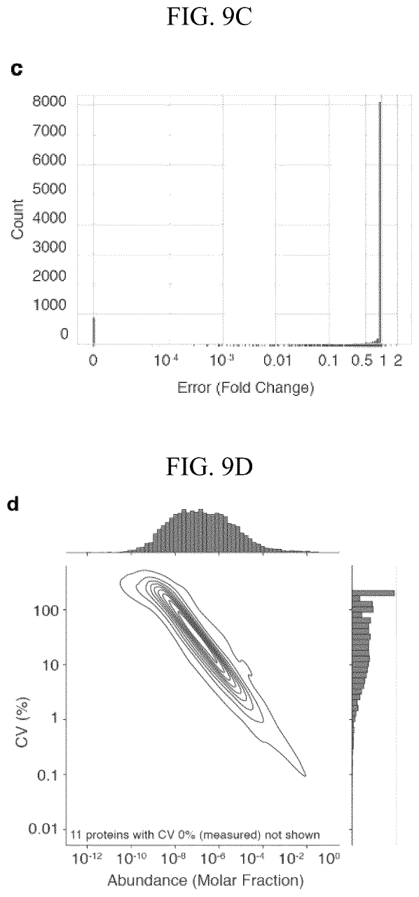

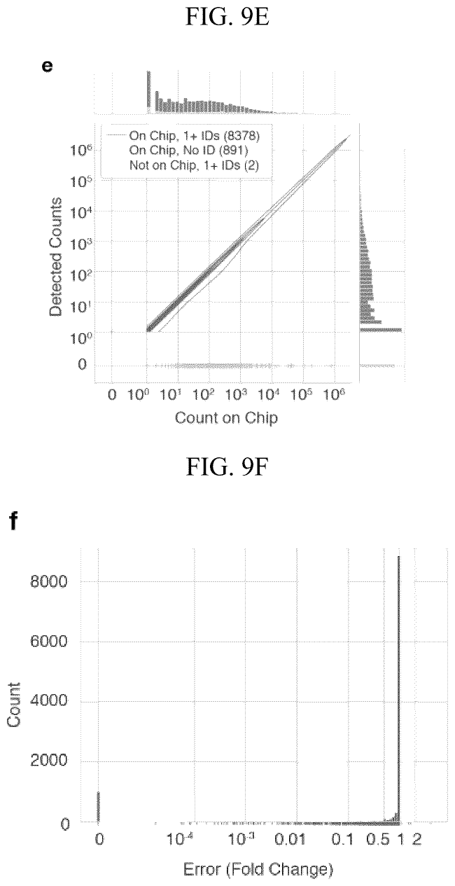

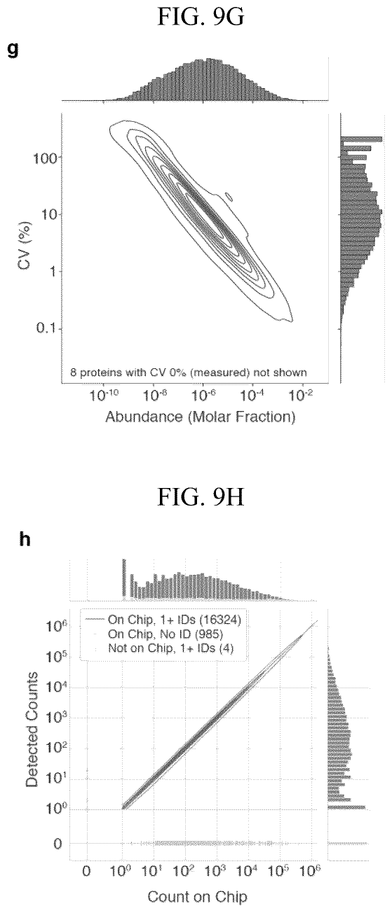

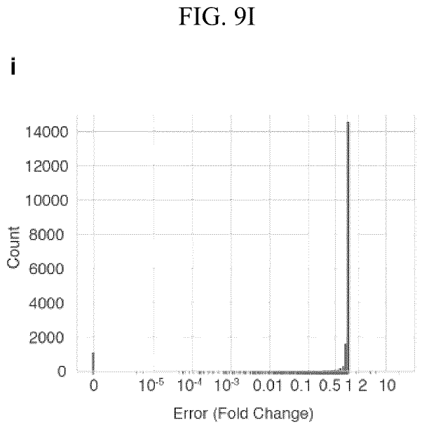

A strategy to increase throughput would be to use an array of 10⁸ protein addresses for each proteome sample (e.g., multiplexing multiple proteome samples on an array or running multiple smaller arrays in parallel). In this situation, the low abundance proteins became undetectable resulting in a compressed dynamic range spanning 7.5 orders of magnitude (for proteins detected consistently) in plasma but with high coverage within that range (FIGS. 9A-9I).

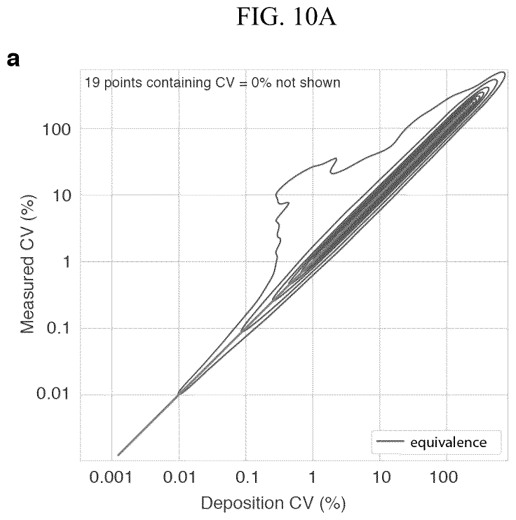

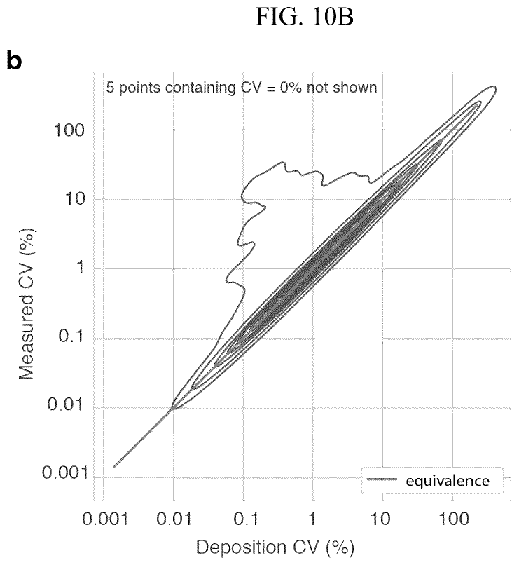

Measurement reproducibility was assessed across the five technical replicates of the modeled blood plasma and HeLa samples (FIGS. 3C & 3D). The coefficient of variation (CV) was <10% for medium to high abundance proteins. Proteins within the top 5 orders of magnitude in terms of abundance in the plasma sample generally had CV <1%. As modeled, the contributors to irreproducibility were stochastic variation in affinity reagent binding and protein deposition as well as variation in affinity reagent binding characteristics. While these estimates do not consider many factors of experimental variability such as sample preparation and biological variability, they demonstrate the potential of the analytic platform and decoding process to contribute minimal variation relative to more common sources of variation. In fact, the CV observed in measurement counts was not much different from the CV of the actual counts, indicating that reproducibility of measurements can be improved by increasing throughput (FIGS. 10A & 10B).

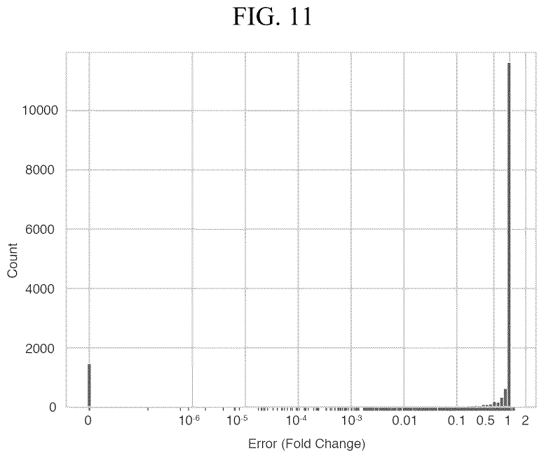

Detected protein counts correlated with the number of proteins modeled on the array (FIGS. 3E & 3F). 76% of plasma proteins had a fold change error in detected counts relative to counts on array within +/−10% (FIG. 11). In some cases, proteins with only a single copy on a chip were detected. Some proteins were substantially under-counted due to sequence similarity to other proteins in the sequence database. The linear nature of detection count vs. counts on array indicated that dynamic range can be extended further by expanding the array to 10¹¹ addresses or evaluating a sample across multiple arrays.

In conclusion, the results presented in this example provide a theoretical foundation for a single-molecule protein identification method that is proteome invariant and can be used to analyze the entire human proteome in a single experiment. It may take a non-destructive affinity reagent approach, rather than a chemically intensive or cleavage-based sequencing approach. It is robust to false negatives (i.e. failure of an affinity reagent to bind its epitope) and is optimized for non-specific affinity reagents. The decoding method is scalable to full proteome quantification and, unlike mass spectrometry, is capable of quantification over a wide dynamic range. By using intact proteins, the decoding method avoids the loss of information (such as proteoforms) that limits approaches that are based on detecting peptide fragments of proteins, and partially mitigates the dynamic range challenge, as sample complexity is decreased by approximately two orders of magnitude.

As the dynamic range of the exemplified decoding method is directly related to the number of intact protein molecules measured, a particularly useful detection system will have rapid imaging and cycle speed. Preliminary estimates suggest that, with 300 affinity reagents and cycle times of approximately 10 minutes, it will be possible to profile ten-billion protein molecules within about a day.

### Methods

### Protein Sequence Databases

Protein sequence databases were downloaded from Uniprot (www.uniprot.org). For each species, the “reference” proteome was selected by including “reference:yes” in the search query string for proteomes. The reference proteome was then filtered to only include Reviewed (Swiss-prot) sequences (query string “reviewed:yes”). The sequence data was then downloaded in uncompressed .fasta format (canonical sequences only). Specific proteomes and filter strings used were:
E. coli (strain K12): reviewed:yes AND organism: “Escherichia coli (strain K12) [83333]” AND proteome:up000000625 (downloaded 6/30/2021)
S. cerevisiae (5288c): reviewed:yes AND organism: “Saccharomyces cerevisiae (strain ATCC 204508/S288c) (Baker's yeast) [559292]” AND proteome:up000002311 (downloaded 6/30/2021)
M. musculus (c57b1): reviewed:yes AND organism: “Mus musculus (Mouse) [10090]” AND proteome:up000000589 (downloaded 6/30/2021)
H. sapiens: reviewed:yes AND organism: “Homo sapiens (Human) [9606]” AND proteome:up000005640 (downloaded 7/6/2021)

The proteomes were further processed to remove any duplicated sequences and any sequences not entirely composed of the 20 canonical amino acids. Further, sequences of length 30 or less were removed from each FASTA.

### Modeling Affinity Reagent to Protein Binding

An affinity reagent targeting epitopes of length k (e.g. for a trimer, k=3) was modeled by assigning a binding probability θ to each unique target epitope j of length k recognized by the reagent. Further, a protein non-specific binding rate was assigned p_{nsbepitope} representing the probability of the affinity reagent binding to any epitope in a protein non-specifically. Given the primary sequence for a protein of length M, the probability of an affinity reagent binding to the protein was computed as follows:

First the probability of a specific binding event happening was computed:

$${p}_{specific} = 1 - {\prod }_{j = 1}^{8000} {( 1 - {θ}_{j} )}^{{x}_{j}}$$

with:
X: the count of each epitope j in the protein sequence
   X={x₁, x₂, x₃ . . . } with xⱼ∈ [symbol] *
θ: the binding model parameters. A vector of probabilities of the affinity reagent binding to each recognized epitope
   θ={θ₁, θ₂, θ₃, . . . } with 0≤θⱼ≤1.

Next, the probability of a non-specific protein binding event happening was computed:

[symbol]

=1−(1− [symbol] )^{M-k+1}

with:
p_{nsbepitope}: the probability of the affinity reagent non-specifically binding to any epitope in the protein

0≤ [symbol] ≤1
M: the length of the protein sequence
k: the length of the linear epitope(s) recognized by the affinity reagent.

The probability of the affinity reagent binding to the protein and generating a detectable signal was the probability of 1 or more specific or non-specific binding events occurring:

[symbol]

=1−(1− [symbol] )*(1− [symbol] )

When noted, the probability of binding to each protein was adjusted to account for additional random surface non-specific binding (NSB). That is, binding of an affinity reagent to the array close enough to the protein address to generate a false-positive binding event. The prevalence of surface NSB is defined as a probability 0≤p_{surfacensb}<1 of such a surface NSB event occurring during the acquisition of a single affinity reagent measurement at a single protein location on the array. The adjusted probability of a protein binding event taking into account surface NSB was:

[symbol]

=1−(1− [symbol] )*(1− [symbol] )

### Biosimilar Affinity Reagent Model

Unless specifically noted, affinity reagents were modeled using a “biosimilar” model. In this model, an affinity reagent targets a specific epitope which it binds with probability 0.5. The affinity reagent also binds nine additional primary off-target epitopes with probability 0.5 that are biosimilar to the targeted epitope. Biosimilar targets were selected by computing a pairwise similarity score of the target epitope to every other possible epitope of the same length. The similarity score was computed by summing up the BLOSUM62 similarity between the pair of residues at each sequence location. For example, if computing the similarity of a trimer SLL with trimer YLH, the score would be BLOSUM62(S,Y)+BLOSUM62(L,L)+BLOSUM62(L,H). With all pairwise similarity scores computed, the top nine most similar epitopes to the target were selected as the primary off-target epitopes. In the case of a tie where multiple potential off-target epitopes have the same score, a random epitope was selected. In addition to the target epitope and four off-target epitopes, up to 20 additional secondary biosimilar off-target epitopes of lower binding probability were added to the affinity reagent. The 20 secondary off-target epitopes bind to the next 20 most biosimilar epitopes beyond the ones already included in the affinity reagent model. These 20 additional epitopes have a probability computed as:

b*(1.5^{ot-ss})

with:
b=binding probability of the affinity reagent to its target,
ot=BLOSUM62 similarity score between affinity reagent target and this off-target epitope,
ss=BLOSUM62 similarity score between affinity reagent target and itself
If any of these additional off-target epitopes had binding probability that was less than the affinity reagent epitope non-specific binding rate, it was not included. The epitope non-specific binding probability was set at 2.45×10⁻⁸.

### Simulation of Stochastic Affinity Reagent Binding

To simulate binding of a series of affinity reagents to a single protein, the binding probability θᵢ of each affinity reagent i to the protein was first determined using the methods described in the Modeling Affinity Reagent to Protein Binding section above. To simulate the outcome of the binding for each affinity reagent, a single random draw was taken from the Bernoulli distribution parameterized by θᵢ. An outcome of 1 is binding, an outcome of 0 is no binding.

### Protein Decoding

### Overview

The protein decoding process analyzed a series of affinity reagent binding measurements acquired on an extant protein and determined the most likely identity of that protein among a set of candidates. The most likely protein identity was the one most compatible with the observed binding measurements. This compatibility was determined based on a binding model for each affinity reagent in the experiment which were used to estimate how likely each affinity reagent was to bind each potential protein. A strong candidate protein was one where most of the observed binding events were consistent with affinity reagents likely to bind that protein. A weak candidate protein will have many instances where binding is observed for affinity reagents that are not expected to bind the candidate. The strongest candidate protein was deemed the most likely identity for the extant protein and confidence in this identification was computed as a relative measure of the compatibility of the most likely protein compared to all the other candidates.

### Inputs

The inputs to the decoding process were:
Binding data: D=[d₁, d₂, d₃ . . . d_{N}] with d∈{0 [symbol] , 1 [symbol] }. A sequence of binding measurements, one for each affinity reagent to an extant protein.
A sequence database of length M containing the primary sequence and name of each potential protein that may be present in the sample (e.g., the human protein sequence database described in section Protein Sequence Databases above)
A parameterized binding model for each of the N affinity reagents used in the experiment (see section Modeling Affinity Reagent to Protein Binding above).
An optional surface non-specific binding rate (r) describing the probability of a surface non-specific binding event happening at any one address in any given cycle.

### Binding Probability Calculations

An M×N binding probability matrix B was computed describing the probability of each affinity reagent binding to every possible candidate protein with an entry in the matrix b_{i,j} being the probability of affinity reagent j binding to candidate protein i. These probabilities were computed using the methods described in the Modeling Affinity Reagent to Protein Binding section above.

Next, the M×N matrix U with adjusted non-binding probabilities for each affinity reagent to each protein was computed as follows:
Compute S=[s₁, s₂, s₃, . . . s_{M}] where sᵢ= [symbol] −2.
Compute F=[f₁, f₂, f₃, . . . f₈₀₀₀] the relative frequency of every possible unique trimer among the set of all candidate protein sequences where:

$${f}_{p} = \frac{{\sum }_{q = 1}^{8000} {trimer}_{q} frequency}{}$$

Compute A=[a₁, a₂, a₃, . . . a_{N}] the vector of average trimer non-binding probabilities for the affinity reagents. A value aⱼ in A is the probability of the affinity reagent not binding to a trimer, averaged over all 8000 trimers and weighted by the relative frequency of each trimer in the candidate protein database aⱼ=Σₚ₌₁⁸⁰⁰⁰fₚ(1−_{p,j})(1−cⱼ) where t_{p,j} is the probability of affinity reagent j binding to trimer p and cⱼ is the probability of a non-specific protein binding event happening for affinity reagent j.
Compute U where u_{i,j}=aⱼ^{s}^{ᵢ}(1−r) is the adjusted probability of affinity reagent j not binding to protein i (r is the surface NSB rate).

Adjusted non-binding probabilities were computed in this manner (as opposed to U=1−B) to avoid any single non-binding event having an outsized impact on a protein. The rationale was that there are numerous difficult to predict reasons why an affinity reagent may not bind to a specific epitope (e.g., protein structure, post-translational modifications) and so the total number of non-binding events should be considered more than the specific identity of the observed non-binding events.

### Decoding

A vector of likelihoods for each protein in the candidate database was computed by multiplying the likelihoods of each observed binding event:

$$L = [ {ℒ}_{1} , {ℒ}_{2} , {ℒ}_{3} … {ℒ}_{M} ] where : \\ {ℒ}_{i} = {\prod }_{j = 1}^{j = N} ( {d}_{j} {b}_{i , j} + ( 1 - {d}_{j} ) {u}_{i , j} )$$

The protein of highest likelihood was selected (if there was a tie for top protein, one of the top proteins is selected randomly):

[symbol]

The probability of the ID being correct is the likelihood of the top protein divided by the sum of the likelihood of all other proteins:

$$= \frac{{ℒ}_{ID}}{{\sum }_{i = 1}^{i = M} {ℒ}_{i}}$$

The protein ID and probability are the outputs for the decoding process performed on a single extant protein.

### Calculation of Proteome Coverage

To compute proteome coverage, a set of affinity reagents was defined as in the Modeling Affinity Reagent to Protein Binding section above. Binding of the affinity reagents was simulated for each protein (see the Simulation of Stochastic Affinity Reagent Binding section above) in the human proteome as defined in the Protein Sequence Databases section above. The binding data was then passed to the decoding process along with the definition of the affinity reagents, and the FASTA sequence database. The output of the decoding process was a single protein identification for each simulated protein and an estimated probability of that identification being correct. To compute the fractional coverage, the number of proteins identified above a true/false discovery rate threshold of 1% (see the Computing and Thresholding on False Discovery Rate section below) was divided by the total number of proteins simulated. The percent coverage was computed by multiplying fractional coverage by 100. This method was applied for all analyses except for modeling of cell, plasma, and depleted plasma samples which use the method described in the Quantitative Statistics section below.

### Computing and Thresholding on False Discovery Rate

Given a list of decoded protein identities (protein identity and associated probability), the false discovery rate was computed by first annotating each protein identification as correct or incorrect based on its match to the true identity of that protein in the simulation. For each unique identification probability in the list, the false discovery rate (FDR) was computed as the fraction of proteins at that probability or lower that were incorrectly identified. To threshold on false discovery rate, the lowest probability score threshold with FDR less than the desired FDR was determined. Identifications at this probability score or higher satisfied the FDR criterion and were considered “identified” at the desired FDR threshold. FDR can also be computed as set forth in Example II, below.

### Demonstration of Stochastic Binding

Stochastic binding of a sequence of 10 affinity reagents to protein EGFR was simulated six times (FIG. 1C). Affinity reagents with binding sequence present in EGFR have a 0.5 probability of binding and those without a binding sequence in EGFR have 0 probability of binding. Binding was simulated as described in the Simulation of Stochastic Affinity Reagent Binding section above.

### Evaluation of Affinity Reagent Requirements for Efficient Decoding

Affinity reagents with various target epitope lengths (2, 3, or 4 i.e., dimer, trimer, tetramer, respectively) with varying numbers of primary off-target epitopes were modeled.

In each case, the target binding probability was 0.5. “Number of Epitopes per Affinity Reagent”=1 represents affinity reagents targeting a single epitope, with no primary off-target epitopes. Other scenarios were modeled with the affinity reagents having some number of primary biosimilar (see the Biosimilar Affinity Reagent Model section above) off-target epitopes. For example, an affinity reagent labeled as targeting ‘5’ epitopes has binding affinity for its target and four primary off-target sites. Affinity reagents did not have any secondary off-target epitopes (see Biosimilar Affinity Reagent Model section above). The targets of affinity reagents were selected randomly from targets present in the proteome. There was no requirement for off-target binding epitopes being present in the proteome.

To determine the number of affinity reagents required to achieve 90% coverage of the proteome, binding of an excess of affinity reagents (i.e., more than required for 90% coverage) was simulated to each protein in the proteome. For any number of affinity reagents N, the proteome coverage was computed using the first N affinity reagents in the set. The number of affinity reagents required to achieve 90% proteome coverage was the lowest N with coverage at or exceeding 90%. The values of N tested were in increments of 10.

With the number of affinity reagents (N) required for 90% coverage computed, the number of binding events observed for each simulated protein was recorded, and the mean of these values reported as the “Average Number of Binding Events per Protein”. Additionally, the percent of proteins generating a binding event for each affinity reagent was recorded, and the mean of these values was reported as the “Percent of Proteins Bound Per Affinity Reagent”.

### Selection and Evaluation of Optimal Affinity Reagent Trimer Targets

The standard biosimilar affinity reagent model (see Biosimilar Affinity Reagent Model section above) was used in this analysis with trimer-targeting affinity reagents. One set of ‘optimal’ affinity reagent targets was computed by using a greedy-selection process to estimate the optimal set of 300 targets to achieve high proteome coverage with as few affinity reagents as possible. Additionally, 20 sets of 300 targets were selected randomly among trimers present in the proteome (excluding any trimers containing a cysteine). Proteome coverage for each of the 21 affinity reagent sets was evaluated as described in the Calculation of Proteome Coverage section above. Proteome coverage was also evaluated for multiple first-N reagent subsets of each affinity reagent set to evaluate scaling of proteome coverage with number of affinity reagents used.

The optimal set of trimer targets was chosen as set forth below:
1. Initialize an empty list of selected affinity reagents (AR).
2. Initialize a set of candidate ARs (e.g., a collection of 6,859 ARs, each targeting a unique trimer without a cysteine in it).
3. Select a set of protein sequences to optimize against (e.g., all human proteins in the UniProt reference proteome).
4. Repeat the following until the desired number of ARs has been selected:
   a. For each candidate AR:
      i. Simulate binding of the candidate AR against the protein set.
      ii. Perform decoding for each protein using the simulated binding measurements from the candidate AR and the simulated binding measurements from all previously selected ARs.
      iii. Calculate a score for the candidate AR by summing up the probability of the correct protein identification for each protein determined by protein inference.
   b. Add the AR with the highest score to the set of selected ARs, and remove it from the candidate AR list.

### Evaluation of Proteome Coverage in Multiple Organisms

Proteome coverage was assessed for four different organisms using the 300 affinity reagents targeting the optimal trimer set (see Selection and Evaluation of Optimal Affinity Reagent Trimer Targets section above) designed against the human proteome. Sequence databases for each organism are described in the Protein Sequence Databases section above. For each organism, binding was simulated using an affinity reagent epitope binding affinity of 0.5 for each affinity reagent against each protein in the sequence database for that organism. The binding data were then decoded using the appropriate sequence database for the organism and proteome coverage assessed as described in the Calculation of Proteome Coverage section using various first-N subsets of the 300 affinity reagent set. For example, to compute coverage at 100 affinity reagents for a given organism, only data from the first 100 of the 300 affinity reagents total were considered when decoding.

### Application of Noise to Affinity Reagent Binding Probabilities

A method was devised to model random perturbations in affinity reagent binding characteristics. The method applied random “noise” to the trimer (or other short linear epitope) binding probabilities while maintaining probabilities bound between 0 and 1. Given a binding probability p a perturbed probability was determined by drawing a sample from the distribution:

Φ(Φ⁻¹)(p)+ [symbol] (0,σ²))

where:
[symbol] is the normal distribution,
σ² is a parameter used to tune the severity of the perturbation, and
Φ is the cumulative distribution function of the standard normal distribution.

The parameter σ² was set such that the mean absolute deviation (MAD) of the distribution divided by the trimer probability p was equal to a desired target. This tuning parameter will be referred to as the “fractional MAD”. The fractional MAD was used to tune the noise due to its conceptual similarity to the coefficient of variation (standard deviation divided by mean) often used to describe measurement noise or reproducibility for normally-distributed measurements.

A numerical approximation method was used to find the value of σ² for a probability p that results in the desired fractional MAD. First, given p and the desired fractional MAD, the target MAD was computed as fractional MAD*p. A function optim is defined which, given p the target MAD, and a proposed σ² value generates 10,000 random samples from the noise distribution parameterized by p and σ² and returns the absolute value of the difference between the MAD of the 10,000 random samples and the target MAD. The minimize_scalar function from the scipy Python package is used to estimate the value of σ² which minimizes this function. This process is repeated 50 times, and the median optimal σ\hat{ }² among the 50 trials is taken as the appropriate value to generate a noise distribution with the desired MAD.

### Modeling of Experimental Confounders

### Poor Binding Affinity

Proteome coverage (see Calculation of Proteome Coverage section above) was assessed using the 300 affinity reagents targeting the optimal trimer set (see Selection and Evaluation of Optimal Affinity Reagent Trimer Targets section above) binding to each unique protein in the human proteome (FIG. 2A). However, the affinity reagents were modeled with a variety of target epitope binding rates ranging from 0.01 to 0.99 to simulate varying affinity reagent binding affinity. Proteome coverage was assessed as described in the Calculation of Proteome Coverage section using various first-N subsets of the 300 affinity reagent set to model the relation between number of affinity reagents used and proteome coverage. Binding simulation and decoding were repeated five times to generate replicate analyses.

### Non-Specific Binding to Array Surface

Proteome coverage was assessed with varying combinations of affinity reagent binding affinity and non-specific binding rate. In every case, 300 affinity reagents targeting the optimal trimer set (see Selection and Evaluation of Optimal Affinity Reagent Trimer Targets section above) were used. However, the affinity reagents were modeled with a variety of target epitope binding rates ranging from 0.05 to 0.95 to simulate varying affinity reagent binding affinity and also varying surface non-specific binding ranging from 0 to 0.3. After modeling binding with surface NSB, proteome coverage was computed as described in the Calculation of Proteome Coverage section above.

### Missed Trimers During Affinity Reagent Characterization

Binding measurements for each of the set of optimal affinity reagents (see Simulation of Stochastic Affinity Reagent Binding section above) were generated against each of the proteins in the human FASTA database (see Protein Sequence Databases section above) with a surface NSB rate of 0.1% (see Non-Specific Binding to Array Surface section above). Prior to decoding the binding measurements to generate protein IDs, the affinity reagent models were corrupted by removing a fraction of primary epitopes. Such a corruption could occur in an experimental setting, for example, if the method used to determine the epitopes that an affinity reagent binds to missed some number of epitopes. The corrupted affinity reagent models were used when decoding the binding measurements to generate protein IDs and were expected to reduce decoding performance. The severity of the corruption was modulated by adjusting the percentage of primary epitopes that were missed. To model 20% of primary epitopes being missed, a random 20% of the primary epitopes (among all affinity reagents collectively) were selected for removal. Because the optimal affinity reagents have ten primary epitopes, this means that on average, two primary epitope was missed in each affinity reagent, although some may have more than one removed and others may have none removed due to random chance. In some analyses, a percentage of secondary epitopes were also removed in a similar manner.

### False Identification of Trimer Epitopes During Affinity Reagent Characterization

Similar to the Missed Trimers During Affinity Reagent Characterization section above, binding of affinity reagents to proteins in the proteome was simulated with surface NSB 0.1% and affinity reagent models were corrupted prior to decoding. For this analysis, false positive epitopes were added to the affinity reagents prior to decoding. This simulates a scenario where the method used to characterize the epitopes bound by each affinity reagent falsely identifies some number of trimer epitopes which the affinity reagent does not bind to. The severity of the corruption was modulated by adding false primary epitopes such that the complete set contained a specific percentage of false epitopes. For example, 20% false epitopes means that false primary epitopes were added until 20% of the primary epitopes among the affinity reagent set were false. The extra epitopes were randomly distributed among the affinity reagents. The trimer identities of the extra epitopes were selected randomly with replacement. In some analyses, secondary epitopes were also impacted by corruption. Any added secondary epitopes should not match an existing or added primary epitope. For example, an affinity reagent targeting the primary epitopes HNW, HDW, and HHW and secondary epitopes HRW, and HGW could have LWW added as either a corrupting primary or secondary epitope, but HGW could only be added as a corrupting primary epitope, in which case its binding probability would be updated to that of a primary epitope.

### Consistent Over- or Under-Estimation of Affinity Reagent Trimer Binding

Similar to the Missed Trimers During Affinity Reagent Characterization section above, binding of affinity reagents to proteins in the proteome was simulated with surface NSB 0.1% and affinity reagent models were corrupted prior to decoding. In this analysis, epitope binding probabilities were adjusted to be systematically higher or lower than the true values. This models a situation where the affinity reagent characterization method determines the correct trimer epitopes targeted by the affinity reagent, but systematically over or under-estimates the strength of binding (modeled by binding probability). The manipulation entailed applying some fold-change shift to the binding probability of the epitopes such that the primary epitopes of the affinity reagent are shifted by a desired amount. For example, to model a shift of +0.25 for an affinity reagent with true primary epitope binding probability of 0.25, the binding probability of every epitope of the affinity reagent was multiplied by 2. In this case, a primary epitope with true binding probability of 0.25 will be assumed to bind with a probability of 0.5 when performing decoding. Similarly, this same multiplicative shift may be applied to secondary binding epitopes. For example, a secondary epitope with binding probability 0.2 would then have binding probability 0.4. Similarly, adjustments may be made which adjust binding probabilities to be less. In some analyses, the severity of the corruption was modulated by only corrupting a fraction of the affinity reagents. For example, 50% of the affinity reagents may be impacted meaning half of the affinity reagents have a systematic error in their binding probabilities while the rest are not impacted.

### Noisy Affinity Reagent Characterization

Similar to the Missed Trimers During Affinity Reagent Characterization section above, binding of affinity reagents to proteins in the proteome was simulated with surface NSB 0.1% and affinity reagent models were corrupted prior to decoding. In this analysis, random noise was applied to the characterized epitope binding probabilities. The random noise was applied to a random fraction of the affinity reagents in the set. For any affinity reagent impacted by noise, all primary and secondary epitopes were subjected to some degree of noise as well as the affinity reagent non-specific binding rate. The binding probabilities were perturbed according to the method described in the Application of Noise to Affinity Reagent Binding Probabilities section above with the amount of noise ranging between fractional MAD 0 and 0.75.

### Simulation of Cell-Line and Plasma Experiments

### Protein Abundance Database Processing

The protein composition of each sample was modeled using protein abundances downloaded from PaxDb v4.1 (Wang et. al., Molecular Cellular Proteomics, 8:492-500 (2012). doi: 10.1074/mcp.O111.014704, which is incorporated herein by reference). Specifically, plasma protein abundances were from the “H. sapiens—Plasma (Integrated)” dataset (https://pax-db.org/downloads/4.1/datasets/9606/9606-PLASMA-integrated.txt downloaded September, 2021). Cell-line abundances were from the dataset “H. sapiens—Cell line, Hela, SC (Nagaraj, MSB, 2011)” (https://pax-db.org/downloads/4.1/datasets/9606/9606-hela_Nagaraj_2011.txt built from high resolution mass spectrometry analysis of HeLa cells (Nagaraj Molecular Systems Biology, 7:548 (2011). doi:10.1038/msb.2011.81, which is incorporated herein by reference). The identities of proteins in the PaxDb data were mapped to the identities of proteins in the Uniprot human protein sequence database (see the Protein Sequence Databases section above) using the PaxDb to Uniprot mapping available from the PaxDb maintainers available at https://pax-db.org/downloads/4.1/mapping_files/uniprot_mappings/full_uniprot_2_paxdb.04.2015.tsv.zip (downloaded September, 2021). Any proteins present in the PaxDb database that could not be mapped to the UniProt sequence database were removed from the sample. 4,342 of 4,492 entries (97%) in the plasma database were successfully mapped with no unmapped protein comprising more than 1% of the sample. 8,554 of 8,817 entries (97%) in the cell database were successfully mapped with no unmapped protein comprising more than 1% of the sample. In some cases, more than one entry in a PaxDb database mapped to a single UniProt identifier in the sequence database. In these cases, only the first entry was retained. In the plasma database, 99 database entries were dropped as a result of this operation (4,243 entries remained). In the cell-line database, 145 entries were dropped (8,409 entries remained). Neither of these operations dropped any entries comprising more than 1% of the corresponding sample. 25, and 97 proteins with abundance 0 were removed from the plasma and cell-line database, respectively. After filtering, the abundance databases were normalized to sum to 1.

### Imputation of Protein Abundances (Plasma)

Abundances were imputed for proteins in the human protein sequence database not represented in the modeled plasma sample (see Protein Abundance Database Processing section above). This process resulted in a ‘complete’ plasma sample containing 20,235 proteins with 12 orders of magnitude in dynamic range of abundance. The distribution of abundances in the complete plasma sample was modeled as a semi-Gaussian distribution (Eriksson, Nature Biotechnology, 25:651-655 (2007). doi:10.1038/nbt1315, which is incorporated herein by reference):

Let f(a|μ, σ) be the normal distribution probability density function with mean μ and standard deviation σ evaluated at x

$$f ( x \vert μ , σ ) = \frac{1}{\sqrt{2 π {σ}^{2}}} exp ( - \frac{{( x - μ )}^{2}}{2 {σ}^{2}} )$$

Let:
Aₘₐₓ be the highest protein abundance in the modeled plasma sample pre-imputation,
σₚ=1.2
μₚ= [symbol] (Aₘₐₓ)−5σₚ
[symbol] = [symbol] (Aₘₐₓ)−12
   Let g(a) be a function proportional to the probability density of the semi-Gaussian distribution at abundance a. g(a)=
f(log₁₀(a)|μ=μₚ, σ=σₚ) if log₁₀(a)≥μₚ
f(μₚ|μ=μₚ, σ=σₚ) if [symbol] ≤log₁₀(a)<μₚ
0 if log₁₀(a)<

[symbol]

Next, a probability density function for abundances of proteins needing to be imputed was estimated. A threshold was set for ‘high-abundance’ proteins t=Aₘₐₓ−4 on the reasoning that any protein with log₁₀(abundance)>t present in the ‘complete’ plasma sample would be accurately represented in PaxDb (i.e., not impacted by detection bias). The probability density of the PaxDb proteins was estimated by computing a histogram (50 bins) on their log−10 transformed abundances and normalizing the values at each bin such that the total area of the histogram is 1.

A scaling factor α was computed to adjust the high-abundance tail of the complete sample abundance distribution g(x) to match the probability density of protein abundances >t in PaxDb:

$$α = \frac{{\sum }_{j} g ( {10}^{{α}_{j}} ) {d}_{j}}{{\sum }_{j} {g ( {10}^{{α}_{j}} )}^{2}}$$

with
{a₀, a₁, a₂, . . . a₁}: the j bin centers of the histogram of log−10 PaxDb abundances with a>t, and
{d₀, d₁, d₂, . . . dⱼ}: the density corresponding to those bin centers.

A kernel density estimate K was fit to the log 10-transformed plasma abundance values using a Gaussian kernel with σ=0.2 and was subtracted from the scaled semi-Gaussian distribution to estimate a function proportional to the density of the probability distribution on abundances for imputed proteins: h(x)=αg(x)−K(x). The function h(x) was evaluated at 500 abundance values spread equally in base-10 log space between log₁₀(Aₘₐₓ)−12 and log 10 abundance log₁₀(Aₘₐₓ). Any points where h(x) evaluated to less than zero were set to zero. A continuous probability distribution was fit to this lattice of sample points using linear interpolation and then normalized such that the total probability of the distribution was 1. The abundances of the 16,017 proteins in the UniProt database not represented in the processed PaxDb dataset were set to random samples from the aforementioned distribution. The resulting abundances are converted to molar fraction estimates by dividing each abundance by the sum of all abundances.

### Imputation of Protein Abundances (Cell-Line)

Abundances were imputed for proteins in the human protein sequence database not represented in the modeled cell-line sample (see Protein Abundance Database Processing section above). This process resulted in a ‘complete’ cell-line sample containing 20,235 proteins with 10 orders of magnitude in dynamic range of abundance. The “complete” cell-line sample was modeled as an adjusted skewed-normal distribution on log 10-transformed abundances:
g(x)=2.45* [symbol] (x|a=−2.12, μ=4.5, σ=2.55)
where skewnorm.pdf is the probability density function of the skewed normal distribution.

A kernel density estimate K (Gaussian kernel, σ=0.2) was fit to the log 10-transformed abundances of all entries in the processed PaxDb database for the cell-line sample. The function h(x) was evaluated at 500 abundance values spread equally in base-10 log space between log 10 abundance log₁₀(Aₘₐₓ)−10 and log 10 abundance log₁₀(Aₘₐₓ). Any points where h(x) evaluated to less than zero were set to zero. A continuous probability distribution was fit to this lattice of sample points using linear interpolation and then normalized such that the total probability of the distribution was 1. The abundances of the 11,923 proteins in the Uniport database not represented in the processed PaxDb dataset were set to random samples from the aforementioned distribution. The resulting abundances are converted to molar fraction estimates by dividing each abundance by the sum of all abundances.

### Depleted Plasma Sample

To model a plasma sample where the most abundant proteins were depleted from the sample (e.g., using a commercially-available affinity column), the abundances of the top-20 most abundant proteins in the imputed plasma sample (see Imputation of Protein Abundances (Plasma) section above) were reduced by 99% and the abundances renormalized to sum to 1 to serve as an estimate of molar fraction.

### Simulating Protein Deposition

Deposition of a sample containing n proteins of abundances {a₁, a₂, a₃, . . . aₙ} on an array was modeled as a multinomial distribution. The protein abundances were normalized to probabilities summing to 1

$${p}_{i} = \frac{{a}_{i}}{{\sum }_{j = 1}^{j = n} {a}_{j}} .$$

To determine the counts of each protein deposited on an array with N addresses, a random sample is made from the multinomial distribution parameterized with the probabilities {p₁, p₂, p₃, . . . pₙ} and N trials.

### Simulation of Binding Data

For each sample type (cell, plasma, depleted plasma), binding was simulated for 5 technical replicate protein arrays. The 300 affinity reagents used for binding targeted the first 300 optimal targets (see Selection and Evaluation of Optimal Affinity Reagent Trimer Targets section above) and used the binding model described in the Biosimilar Affinity Reagent Model section above with a surface non-specific binding rate of 0.001. To simulate random variation in binding replicate-to-replicate, the binding probabilities of affinity reagents were perturbed for each replicate using the method described in the Application of Noise to Affinity Reagent Binding Probabilities section above with fractional mean absolute deviation 0.1. Binding for each flow cell was then simulated as described in the Simulation of Stochastic Affinity Reagent Binding section above.

### Decoding of Binding Data

Protein decoding was performed individually for each replicate as described in the Protein Decoding section above. The human FASTA sequence database (see Protein Sequence Databases section above) was used to define protein candidate sequences. The affinity reagent model used for decoding of all replicates was the original affinity reagent set referenced in the Simulation of Binding Data section above prior to application of random noise. The decoding method assumed a surface non-specific binding rate of 0.001.

### Determining a Probability Threshold for Protein Quantification

At a given identification probability threshold Pt, proteins in samples may be quantified by counting the number of identifications for that protein in the decoding output with probability p>pₜt. However, if the probability threshold is set too low, many false positive identifications may occur resulting in low quantitative specificity. If the probability threshold is set too high, false negative identifications may occur, resulting in low quantitative sensitivity. For each replicate flow cell analyzed, decoding results were processed with probability thresholds: log(p)=0, −1×10\hat{ }(−20), −1×10\hat{ }(−16), −1×10″−14, −1×10″−12, −1×10″−11, −1×10\hat{ }−10, −1×10″−9, −1×10\hat{ }−8, −1×10\hat{ }−7, −1×10\hat{ }−6, −1×10\hat{ }−5, −1×10\hat{ }−4, −1×10\hat{ }−3, −1×10\hat{ }−2, −0.1, −0.2, and −0.3.

For each threshold evaluated:
For every unique protein identified at least once in the dataset:
   Compute the number of reported identifications for the protein that were true positive (i.e., correct identifications) and false positives (i.e., spots incorrectly identified as the protein)
   Compute the specificity of quantification for this protein:

$$\frac{\#}{\#}$$

If the protein has specificity <0.9, label it as non-specific identification
Compute the ‘non-specific detection rate’: the fraction of proteins that fall into the “non-specific detection” class

The lowest threshold value resulting in a non-specific detection rate <0.1% for every replicate analyzed was used for downstream quantification analyses.

### Quantitative Statistics

After thresholding by identification probability, the following statistics were computed for each analysis:
Specificity of protein identification was computed as described in the Determining a Probability Threshold for Protein Quantification section above.
Proteins with at least 1 identification in a given replicate were deemed ‘identified’ in that replicate.
Proteome coverage for a replicate was the percentage of proteins identified at least once in the replicate among all proteins present in the sample.
Reproducibility of quantification (CV %) for a protein across replicates was computed using the number of counts for that protein in each replicate:

$$100 \times \frac{}{} .$$

Proteins not identified in a replicate were assigned count 0.

### Example II

### Evaluating False Discovery Statistics for a Protein Identification Assay Using Pseudo Outcome Profiles

This example describes a method to estimate the false discovery statistics for binding assay measurements in-silico. The method can be performed in the absence of perfect confidence in calculated probabilities for binding of affinity reagents to proteins. The strategy presented here allows for estimation of false identification rate (FIR) and can further be used to determine the distribution of false identifications for a data set including a variety of different proteins and affinity reagents. The strategy can also be used to determine false detection rate (FDR) for the binding assay.

### Overview

A multiplex binding assay is performed in which multiple different affinity reagents are evaluated with respect to binding a plurality of different unknown proteins (i.e. proteins being referred to as ‘unknown’ because it is not known if they are present in the assay even though their sequences would be recognizable based on known or suspected protein content of the sample from which they are derived). The unknown proteins can be identified by a decoding process. Exemplary assays and decoding processes are set forth in Example I.

For a database having 20,000 candidate proteins, a pseudo outcome profile is generated for each of the candidate proteins. Each of the pseudo outcome profiles can be thought of as representing a pseudo protein that is paired with a respective candidate protein, the latter being referred to as the target protein for the paired pseudo protein. The paired pseudo protein is contrived and is not present in the sample from which the candidate proteins are derived. Pseudo proteins are not only contrived, but the amino acid sequences for the pseudo proteins need not be known nor do any amino acid sequence or any other biochemical characteristics of the pseudo proteins necessarily need to be derivable from the pseudo outcome profiles. A paired pseudo protein can be considered to be as similar to its target protein as it is to the next nearest candidate protein in terms binding to affinity reagents, and this can be the case independent of any knowledge of the structure of the pseudo protein.

Two approaches for generating pseudo outcome profiles are set forth in this example. The approaches can be understood at least conceptually in the context of evaluating two candidate proteins in a family (i.e. the proteins having somewhat similar sequences) JARRETT and JAMIE for an extant protein that is identified as JARRETT, for example, using the decoding method of Example I. In the first approach the PSEUDOJARRETT pseudoprotein is derived from the JARRETT candidate protein, wherein PSEUDOJARRETT is the same distance away from JARRETT as the JAMIE protein is from JARRETT. The similarity matrix between these candidate proteins and the pseudo proteins can be represented in the matrix of sequence distances below, wherein lower number indicates closer distance:

| | JARRETT | JAMIE |
|---|---|---|
| JARRETT | | |
| JAMIE | 2 | |
| PSEUDOJARRETT | 2 | 4 |

In the second approach, the PSEUDOJARRETT pseudoprotein is derived from the JAMIE candidate protein, wherein the PSEUDOJARRETT pseudoprotein is the same distance away from JAMIE as the JAMIE protein is from JARRETT. The similarity matrix would be:

| | JARRETT | JAMIE |
|---|---|---|
| JARRETT | | |
| JAMIE | 2 | |
| PSEUDOJARRETT | 4 | 2 |

For either approach, if a significant number of PSEUDOJARRETT identifications are made, then it can be concluded that there is a significant chance of the extant protein being misidentified.

### Generation of Pseudo Outcome Profiles (Approach I:)

$$E G F R {\to }^{Δ} N E X T$$

For each candidate protein in the database, a probability for binding each affinity reagent (AR) is computed. The candidate outcome profile for EGFR binding to nine different ARs is exemplified by the vector below. The values indicate the probability that the respective AR will bind to the EGFR candidate protein in the assay.

| | AR1 | AR2 | AR3 | AR4 | AR5 | AR6 | AR7 | AR8 | AR9 |
|---|---|---|---|---|---|---|---|---|---|
| EGFR | 0.25 | 0.001 | 0.001 | 0.2 | 0.7 | 0.0001 | 0.0001 | 0.002 | 0.3 |

Next, digital vectors are created for all candidate proteins in the database by converting probabilities into binary values using an arbitrary threshold for positive binding outcome vs. negative binding outcome. For example, the threshold for binding can be set at 0.2 such that elements of the first vector having probabilities 0.2 are converted to 1 in the digital vector and elements having probabilities <0.2 are converted to 0 in the digital vector. Other binary symbols can be used in the digital vector. If desired, the elements of the first vector can be converted into more than two categories, such as ternary categories indicating ‘strong binding,’ ‘weak binding,’ or ‘no binding.’ Thus, the radix for the elements of the digital vector can be ternary, quaternary or higher. Moreover, the digital vector can be created based on categorizations other than probability of binding or strength of binding, in accordance with characteristics of measurements obtained by a given assay.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |

The digital vector that is closest the digital vector for EGFR is identified. Closeness is determined based on the Hamming distance between binarized probability vectors in this example. Accordingly, a comparison of amino acid sequences is not necessary for the determination. Rather, vectors representing binding of the proteins to affinity reagents are compared. In this example, the digital vector for the NEXT protein is found to have the closest Hamming distance to the digital vector for the EGFR protein. Accordingly, the NEXT protein is identified as the next closest protein (NCP) to the EGFR protein.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| NEXT | 1 | 1 | 0 | 1 | 0 | 0 | 1 | 0 | 0 |

The difference between the binary probability values for the above EGFR digital vector and the NEXT digital vector is calculated for each AR column to produce a difference vector (DIFF), as exemplified below.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| DIFF | 0 | +1 | 0 | 0 | −1 | 0 | +1 | 0 | −1 |
| NEXT | 1 | 1 | 0 | 1 | 0 | 0 | 1 | 0 | 0 |

The difference vector is rearranged. In the example below, the binary probability values in the DIFF vector are shuffled to produce the SHUFFDIFF vector. The binary values of the SHUFFDIFF vector are added to the values the EGFR vector to produce a pseudo vector paired with the EGFR vector (identified as the PSEUDOEGFR vector below). The shuffle operation can be modified with particular rules. For example, elements of the DIFF vector having a +1 value are only allowed to match up with elements of the EGFR vector having a 0 value, and elements of the DIFF vector having a −1 value are only allowed to match up with elements of the EGFR vector having a 1 value. As a further option, differences in the SHUFFDIFF vector are not allowed to line up with any difference in the DIFF vector. It will be understood that edge cases may arise where the nearest protein to a given protein has three −1 DIFF values, and EGFR only has three 1 values, in which case a pseudo vector will not be generated.

Other rearrangements can be used instead of shuffling individual AR probability values. For example, the linear order of the binary probability values in the DIFF vector can be reversed, or the DIFF vector can be fragmented into strings, each string including two or more binary probability values, and one or more of the strings can be shuffled or reversed.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| DIFF | 0 | +1 | 0 | 0 | −1 | 0 | +1 | 0 | −1 |
| NEXT | 1 | 1 | 0 | 1 | 0 | 0 | 1 | 0 | 0 |
| SHUFFDIFF | 0 | 0 | 0 | −1 | −1 | +1 | 0 | +1 | 0 |
| PSEUDOEGFR | 1 | 0 | 0 | 0 | 0 | 1 | 0 | 1 | 1 |

Binding probabilities for the PSEUDOEGFR vector (PSEUDOEGFR(probs)) are determined by a two-step shuffle: (1) shuffling the probabilities from the EGFR vector (EGFR(probs)) among the AR columns that were classed as “1” in the binarized EGFR vector (EGFR) and (2) shuffling the probabilities from the EGFR vector (EGFR(probs)) among the AR columns that were classed as “0” in the binarized EGFR vector (EGFR). Exemplary results are shown below.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| EGFR (probs) | 0.25 | 0.001 | 0.001 | 0.2 | 0.7 | 0.0001 | 0.0001 | 0.002 | 0.3 |
| PSEUDOEGFR | 1 | 0 | 0 | 0 | 0 | 1 | 0 | 1 | 1 |
| PSEUDOEGFR | 0.7 | 0.002 | 0.0001 | 0.001 | 0.0001 | 0.25 | 0.001 | 0.3 | 0.2 |
| (probs) | | | | | | | | | |

### Generation of Pseudo Outcome Profiles (Approach II:)

$$NEXT {\to }^{Δ} EGFR$$

For each candidate protein in the database, a vector of probabilities for binding each affinity reagent (AR) is computed, the vector is converted to binary values based on a threshold of 0.2, and the NEXT protein is identified as the closest candidate protein in a database search as set forth above for Approach I.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| NEXT | 1 | 1 | 0 | 1 | 0 | 0 | 1 | 0 | 0 |

The changes in binding events to convert the NEXT digital vector to the EGFR digital vector is computed to generate the NEXT>EGFR vector.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| NEXT | 1 | 1 | 0 | 1 | 0 | 0 | 1 | 0 | 0 |
| NEXT > EGFR | 0 | −1 | 0 | 0 | +1 | 0 | −1 | 0 | +1 |

The NEXT>EGFR vector is rearranged to produce the SHUFFDIFF vector and the SHUFFDIFF vector is applied to the NEXT vector to generate the PSEUDOEGFR vector as shown below:

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| NEXT | 1 | 1 | 0 | 1 | 0 | 0 | 1 | 0 | 0 |
| NEXT > EGFR | 0 | −1 | 0 | 0 | +1 | 0 | −1 | 0 | +1 |
| SHUFFDIFF | −1 | 0 | +1 | −1 | 0 | +1 | 0 | 0 | 0 |
| PSEUDOEGFR | 0 | 1 | 1 | 0 | 0 | 1 | 1 | 0 | 0 |

As with Approach I, other rearrangements can be used instead of shuffling individual AR probability values. For example, the linear order of the binary probability values in the DIFF vector can be reversed, or the DIFF vector can be fragmented into strings, each string including two or more binary probability values, and one or more of the strings can be shuffled or reversed.

Binding probabilities for the PSEUDOEGFR vector (PSEUDOEGFR(probs)) are determined by a two-step shuffle to randomly distribute probabilities from EGFR classed as “1” (binding) and those classed as “0” (non-binding). Exemplary results are shown below.

| | AR1 | AR2 | AR3 | AR4 | AR5 | AR6 | AR7 | AR8 | AR9 |
|---|---|---|---|---|---|---|---|---|---|
| EGFR | 1 | 0 | 0 | 1 | 1 | 0 | 0 | 0 | 1 |
| EGFR (probs) | 0.25 | 0.001 | 0.001 | 0.2 | 0.7 | 0.0001 | 0.0001 | 0.002 | 0.3 |
| PSEUDOEGFR | 0 | 1 | 1 | 0 | 0 | 1 | 1 | 0 | 0 |
| PSEUDOEGFR | 0.002 | 0.7 | 0.25 | 0.001 | 0.001 | 0.3 | 0.2 | 0.001 | 0.001 |
| (probs) | | | | | | | | | |

The approaches exemplified above can be used to generate a pseudo outcome profile for any candidate protein. An advantage of the method is that pseudo outcome profiles can be generated for candidate proteins without employing amino acid sequence comparisons. Rather, comparisons are based upon assay results such as binding affinities between proteins and affinity reagents.

Searching with Pseudo Outcome Profiles

In a first option, a pseudo outcome profile is generated for individual candidate proteins in a database. Either Approach I or Approach II can be used as exemplified above. For example, a database including 20,000 candidate proteins that included 20,000 candidate outcome profiles would be expanded to include an additional 20,000 pseudo outcome profiles bringing the total number of binding profiles to 40,000. By way of analogy, a database having 20,000 candidate proteins could be considered to include 40,000 proteins including the 20,000 candidate proteins and 20,000 pseudo proteins (i.e. a paired pseudo protein for each candidate protein). Decoding is carried out using the 40,000 binding profiles (i.e. candidate outcome profiles and pseudo outcome profiles are used together in the decoding process), for example, as set forth in Example I. The number of extant proteins that are identified as a pseudo protein is determined. For example, an extant protein having a binding profile that is more compatible with a pseudo outcome profile than any candidate outcome profile is indicative of a false identification. The fraction or percent of pseudo proteins identified as a function of all proteins identified provides an aggregate estimate of the overall false discovery rate for the assay. Alternatively or additionally, the distribution of pseudo proteins identified is indicative of skew or bias in the identifications obtained from the assay.

In a second option, again using pseudo outcome profiles generated by either Approach I or Approach II, decoding of extant proteins is carried out using the 20,000 candidate outcome profiles (without including the pseudo outcome profiles). The candidate protein having the top identification and probability is identified for each extant protein. Decoding of the extant proteins is also carried out separately using the pseudo outcome profiles (without including the candidate outcome profiles). The pseudo protein having the top identification and probability is identified for each extant protein. The number and/or distribution of extant proteins that have a higher probability of being a pseudo protein compared to the probability of being a candidate protein is determined. The fraction or percent of pseudo proteins identified as a function of all proteins identified can be used to estimate the overall false identification rate for the assay or the overall false detection rate for the assay. Optionally, a confidence or probability threshold can be used. For example, a confidence threshold can be applied such that one can determine a false identification rate for the subset of addresses in an array for which protein identifications are above the threshold. Conversely, one can determine an appropriate confidence threshold to yield protein identifications for a set of addresses such that the false identification rate is below a particular false identification rate. The distribution of pseudo proteins identified can be used to identify any skew or bias in the identifications obtained from the assay. The second option is useful for preventing pseudo outcome profiles from competing with candidate outcome profiles during decoding.

### Evaluating Cross-Talk

Mis-identification of one protein as another can have the pernicious effect of leading to the corruption of quantitative signal for one protein arising from misidentification of some other protein. Pseudo outcome profiles can be used to estimate tendency for failure modes, such as those failure modes that are most likely to occur. Taking the example of the EGFR and NEXT proteins, the PSEUDOEGFR protein can be considered as an estimate of the NEXT protein being falsely identified. Accordingly, the number of extant proteins that are identified as being the PSEUDOEGFR protein can be compared to the number of extant proteins that are identified as being the NEXT protein. The fraction or percent of PSEUDOEGFR identifications provides an estimate of the fraction or percent of NEXT protein identifications that are incorrect. This provides an estimate of the specificity of the assay and decoding process for the NEXT protein.

Pseudo outcome profiles can be generated for any or all pairs of candidate proteins that are suspected of being prone to mis-identification due to signal corruption under the assay conditions or decoding parameters employed. The pseudo outcome profiles can be used to evaluate or determine appropriate pairing of proteins. Protein pairs can be identified based on a variety of characteristics or thresholds. For example, protein pairs for which less than 10 affinity reagents are expected to have a different binding outcome can be selected and pseudo outcome profiles can be created for those pairs. The threshold of 10 expected differences is exemplary and a different threshold can be determined, for example, based on the number and complexity of affinity reagents used. For example, a lower threshold can be selected when using fewer affinity reagents and a larger threshold can be selected when using more affinity reagents. Conversely, a higher threshold can be selected when using fewer affinity reagents and a lower threshold can be selected when using more affinity reagents. Accordingly, the threshold can be set to be at least 2, 3, 5, 10, 15, 20 or more expected differences. Alternatively or additionally, the threshold can be set to be at most 20, 15, 10, 5, 3, 2 or 1 expected differences. Expected differences can also be identified using known methods for comparing protein sequences or based on simulations of binding between protein sequences and affinity reagents.

### Example III

### Evaluation of Decoding Results

The decoding process set forth in Example I is configured to provide comprehensive proteome quantification at single-molecule sensitivity. Computational models that demonstrate the feasibility of decoding are presented in Examples I and II above, and are also presented in this example. Briefly, decoding can be configured to acquire sequential affinity reagent binding measurements on single, full-length, protein molecules. Counterintuitively, using affinity reagents with “poor” specificity (e.g. cross-reacting to many proteins) enables identification of tens of thousands of proteins with only hundreds of affinity reagents. In the simulations set forth in Example I, decoding is able to identify more than 98% of proteins in various species using 300 affinity reagents targeting short trimer epitopes. The decoding process is robust to experimental confounders including non-specific binding and poor binding affinity. Simulations of the approach with an array containing 10 billion protein molecules measured in parallel show a dynamic range of detection of up to 9.5 and 11.5 orders of magnitude for HeLa cells and plasma, respectively.

As demonstrated in Example I, the protein decoding process tolerates stochasticity by asking: “are the positive binding measurements overwhelmingly consistent with just one protein?” The process does not need to rely on any one binding pattern. Moreover, the decoding process is capable of greater than 95% proteome coverage in the presence of experimental confounders. For example, low specificity affinity reagents are useful as demonstrated by FIG. 1D. Over 95% of several proteomes can be decoded using as few as 170 low specificity affinity reagents as demonstrated by FIG. 1F. Furthermore, FIG. 2A demonstrates that over 95% of proteome coverage is attainable even with an 85% false negative binding rate. As demonstrated by FIG. 2B, non-specific binding events are well tolerated by the decoding process.

The results of Example I, indicate a potential for unprecedented depth of proteome coverage using an array having 10 billion addresses. As demonstrated by FIGS. 3A and 3B, 93% of proteins present in a HeLa sample are detectable with a 10-billion address array. This constitutes near-comprehensive coverage of all proteins in the top 9 orders of magnitude dynamic range in plasma. As demonstrated by FIGS. 3D and 3F, a majority of proteins have a coefficient of variation (CV) less than 10%, and 85% of quantified proteins have less than 10% error in counted quantity.

Informational entropy was computed for individual addresses as binding information accumulated in simulated data decoding HeLa lysate with affinity reagents having a 50% epitope binding rate. Entropy was computed as

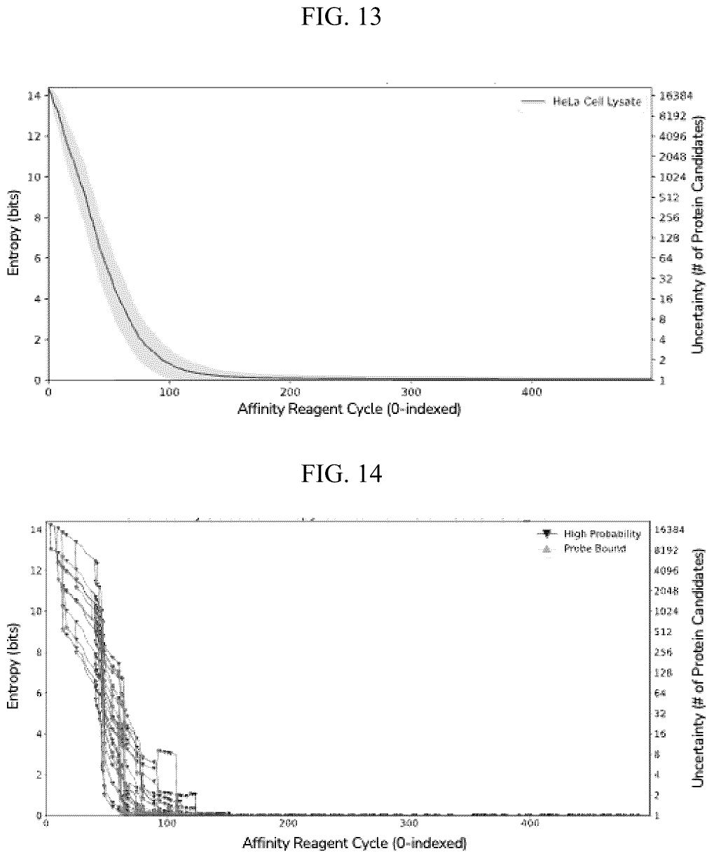

−Σ_{x∈X}p(x)log₂p(x)

where X is all candidate proteins in the FASTA database and p(x) is the computed probability of protein x given the observed binding data. The median entropy decode across all decoded proteins was plotted as shown in FIG. 13. Decode trajectories were plotted for 25 individual addresses each resulting in an identification of 40S ribosomal protein S13, as shown in FIG. 14. High probability binding events (>0.04) and observed binding events are indicated by down-pointing and up-pointing triangles, respectively.

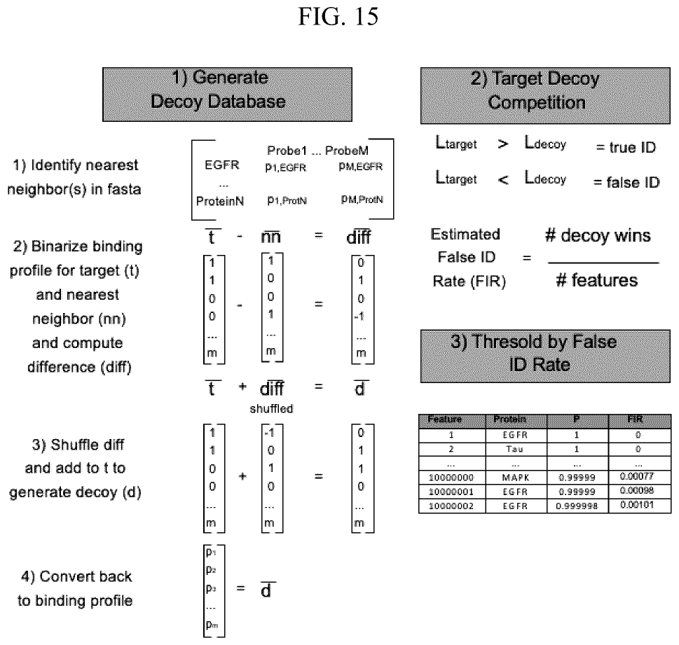

In simulated data, the true identity of each protein is known, and the false identification rate (“FIR”, % of proteins that are incorrectly identified) is trivially computed. When processing data from real experiments, a method to estimate the FIR for a set of protein identifications is beneficial. A useful estimation approach was devised using pseudo binding profiles as “decoys” during decoding. The pseudo binding profiles were not generated from any actual protein but were generated to have similar identification to the target proteins such that the rate of decoy identification is related to the rate of false target protein identification. FIG. 15 provides a diagram of a three step process for generating pseudo binding profiles (“decoys”) and using the pseudo binding profiles to determine false identification rates (“False ID Rate”) at addresses of an array (“features”) detected by a series of for affinity reagents (“probes”). In step 1) a decoy database was generated by identifying the nearest neighbor(s) in the fasta file based on protein sequence and probe binding probabilities. For each nearest neighbor, binding profiles were binarized for the target (t) and nearest neighbor (nn) and the difference (diff) between the two vectors was computed. The difference was then shuffled and added back to the t binding profile to mimic the binding profile of nn. Lastly, binding probabilities were assigned to each probe in the decoy based on the profile of the nearest neighbor. In step 2), for all features, databases for targets and decoys were separately searched. If the top decoy had a higher likelihood match to the binding data than the top target constituted a “decoy win”. At step 3), features were then ranked by the identification quality score (P) and the false identification rate was calculated as the number of decoy wins divided by the number of features. Decoy wins were then removed from the list and all identifications above a user-defined threshold (e.g. 0.001) were accepted.

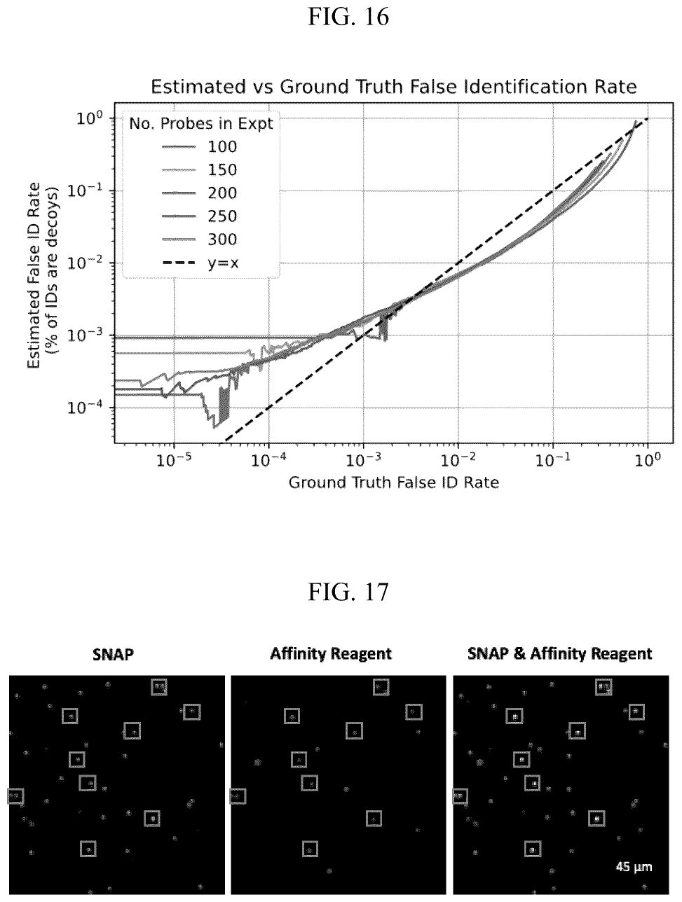

FIG. 16 shows a comparison of estimated FIR to true FIR in simulated cell lysate data. The ground-truth FIR (known from the simulation) was compared to the target-decoy estimated FIR at various identification quality score thresholds. The FIR was slightly over-estimated when the ground-truth FIR was less than 0.001 (0.1%) which means that the process was relatively conservative in making calls on identifications/detections below this rate.

The results presented in this example taken together with Example I indicate that the methods set forth herein can deliver single-molecule proteomics utilizing (1) a hyper-dense single-molecule protein array, (2) highly-parallel measurement of binding to single protein molecules by hundreds of short-epitope affinity reagents via fluorescent imaging, and (3) a protein decoding process built for noisy, stochastic, single-molecule binding data. Moreover, experimental modeling indicated that the methods can deliver near-comprehensive proteomics across nine or more orders of magnitude dynamic range.

### Example IV

### Identification of a Model Protein by Decoding

This example demonstrates identification of a model protein using the decoding method set forth in Example I.

Protein detection data was acquired as set forth in Example I, with the following modifications. Structured nucleic acid particles containing a model protein (WNKKFRYFRRFRFWDDSDFHHHTGRTWTVPFHRHRETYRLTATDRGRWFH) (SEQ ID NO: 1) were deposited onto an array surface in a first lane of a flow cell and structured nucleic acid particles lacking proteins were deposited onto an array surface in a second lane of the flow cell. The structured nucleic acid particles were annealed to addresses on the array as set forth in Aksel et al., BioRxiv High-density and scalable protein arrays for single-molecule proteomic studies (2022) doi.org/10.1101/2022.05.02.490328, which is incorporated herein by reference. The flow cell lanes were then subjected to 30 to 75 protein detection cycles, each cycle including steps of (a) incubation with 5-10 nM fluorescently-labeled affinity reagents, (b) rinsing to remove unbound affinity reagents, (c) fluorescence imaging in a 1500×1500 center pixel region to detect bound affinity reagents, (d) stripping the array with 10% sodium dodecyl sulfate to remove bound affinity reagents and (e) a second fluorescence image was acquired from the 1500×1500 center pixel region. Fluorescence images were acquired in two channels, a first channel that detected fluorophores attached to the structured nucleic acid particles and a second channel that detected the fluorescently-labeled affinity reagents. An aptamer with a scrambled DNA sequence was delivered in one cycle as a negative control.

FIG. 17 shows images acquired from a region of the array in the first lane of the flow cell. The left panel shows images obtained from the array region using a fluorescent channel tuned to detect fluorescent labels on the SNAPs. The images demonstrate that the SNAPs were individually resolvable on the array. The middle panel shows images obtained from the same region using a fluorescent channel tuned to detect fluorescently labeled affinity reagents that bound to the model protein attached to the SNAPs. The results demonstrated that affinity reagents, when bound to a single protein molecule at a given SNAP, were detectable. The right panel shows a composite of the images shown in the left and middle panels, and blue boxes indicate co-localized SNAPs and bound affinity reagents.

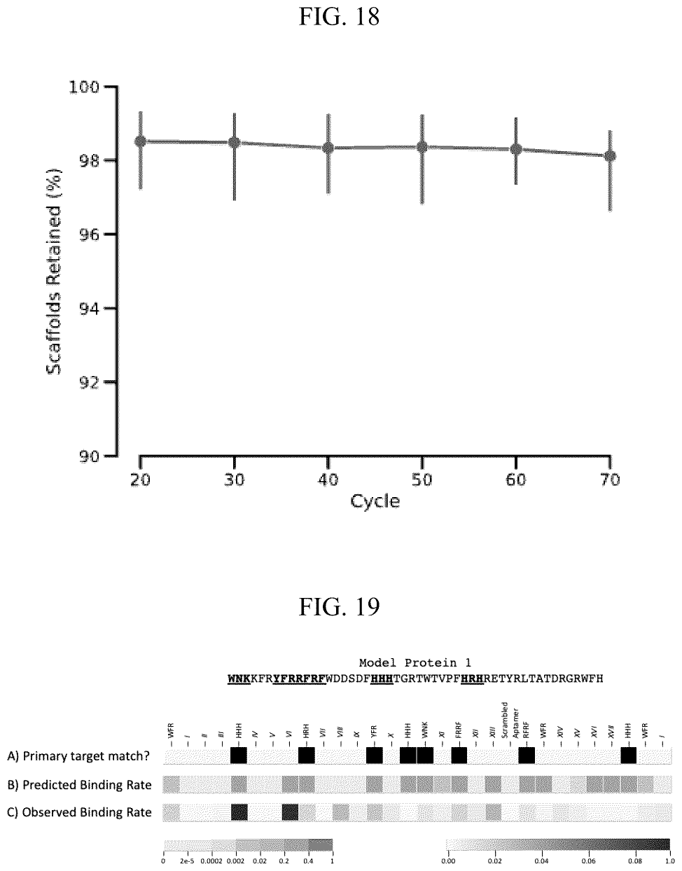

Retention of SNAPs was measured at each cycle and plotted in FIG. 18 as the percent of SNAPs remaining relative to the amount present at the tenth cycle. The median retention rate and inner 95^{th} percentile retention rate are shown in the figure.

The amino acid sequence for model protein 1 with epitope targets underlined is shown at the top of FIG. 19. Rows A) through C) include boxes that are shaded to indicate characteristics for each affinity reagent as follows. The affinity reagents corresponding to each box are identified above Row A) by their primary epitopes or by roman numerals. Row A) indicates the presence (black) or absence (grey) of a primary epitope for each of the thirty affinity reagents that led to successful decoding of Model Protein 1. Row B) indicates predicted binding rates for each of the thirty affinity reagents. Affinity reagents that are predicted to bind at a high rate (e.g. HRH) are indicated by darker shading, while others having low rates (e.g. XV) are indicated by lighter shading. The shading gradient for Row B) is shown at the lower left of FIG. 19. Each affinity reagent is predicted to bind many biosimilar target epitopes. Row C) indicates the observed difference in binding rates between the model protein 1 in lane 1 of the flow cell minus the negative controls in lane 2 of the flow cell (empty SNAPS) across the thirty affinity reagents. Darker shading reflects elevated binding rates of the affinity reagents in the model protein 1 sample, whereas lighter shading indicates lower binding rates. The shading gradient for Row C) is shown at the lower right of FIG. 19. Binding rate is defined as the overall count of SNAPS observed to have a positive binding event in a given lane divided by the total number of SNAPS present in the lane.

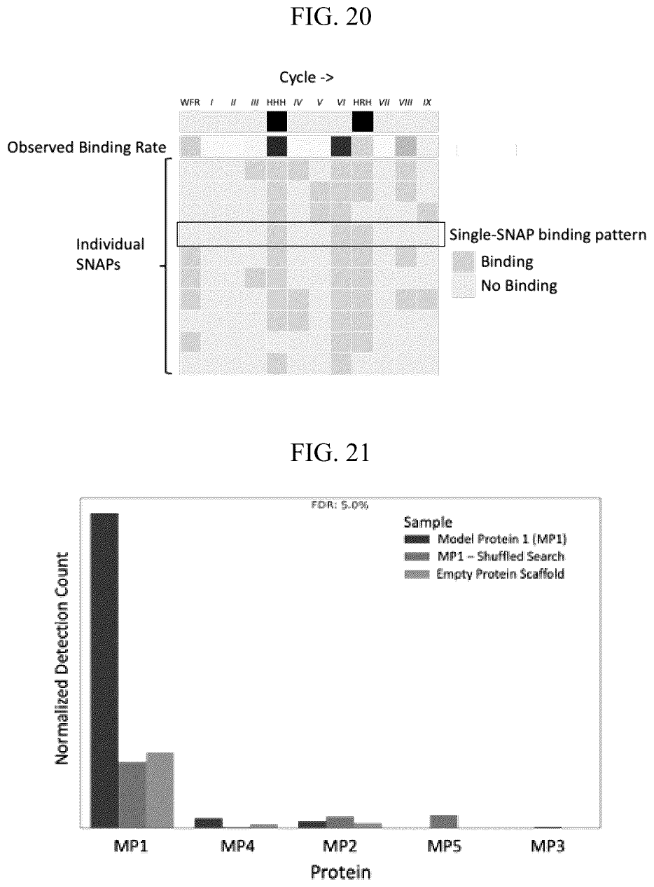

Binding patterns for a subset of individual SNAPs in the array were correctly identified as model protein 1. FIG. 20 depicts individual binding events for ten individual SNAPs in lane 1 that were identified as presenting model protein 1. For clarity, only the first twelve cycles are shown. The rows labeled as “primary target match?” and “Observed Binding Rate” are configured as described above for FIG. 19. The rows labeled as “individual SNAPs” use a binary representation of observed binding events (dark) or observed non-binding events (light) in each cycle. Decoding was performed as described in Example I, however using a candidate protein database that included model protein 1 (MP1) along with four other candidate proteins (MP2, MP3, MP4 and MP5). FIG. 21 shows a bar chart of normalized detection counts for the proteins. The relative count of model protein 1 (MP1) identifications generated by decoding is shown for (1) a sample containing model protein 1, (2) the same sample decoded with the order of affinity reagents being shuffled in-silico prior to decoding (See Example III), and (3) the “empty protein scaffold” negative control (i.e. SNAPs in lane 2 with no attached protein). Model protein 1 was the most frequently identified protein compared to controls (2) and (3). The false discovery rate (FDR), calculated as the percentage of proteins above a given decode score threshold that were identified as proteins other than model protein 1 in lane 1, was 5.0%. The same threshold was applied to all analyses. The results indicated that 90% of the identifications matched model protein 1. The shuffled data verified that identifications were not an artifact of spurious binding.

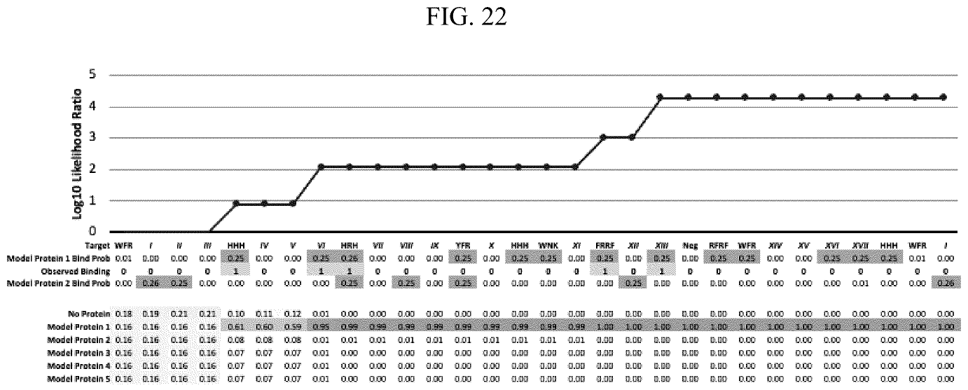

FIG. 22 plots the log 10 likelihood ratio for the SNAPs in lane 1 of the flow cell being identified as model protein 1 for each decoded cycle. The points on the plotted line correlate with the affinity reagent delivered in respective cycles (identified on the x axis by primary epitope targets or roman numerals). Listed in the columns below the respective cycles are the a priori binding probabilities for the respective affinity reagent binding to model protein 1 (“Model Protein 1 Bind Prob”); the observed binding indicated by binary values with “1” indicating binding observed above a threshold value, and “0” indicating absence of binding observed above the threshold value; and the a priori binding probabilities for the respective affinity reagent binding to model protein 2 (“Model Protein 2 Bind Prob”). Model protein 2 was selected as a decoding control because it was not present in the array and because it has the closest a priori binding profile compared to the a priori binding profile for model protein 1. The likelihoods for each protein in the test database being identified as the protein attached to the SNAPs in lane 1 of the flow cell are listed in the lower 5 columns of the figure. The results obtained from all cycles indicated that MP1 was 100,000 times more likely to be the correct identification compared to the MP2 negative control.

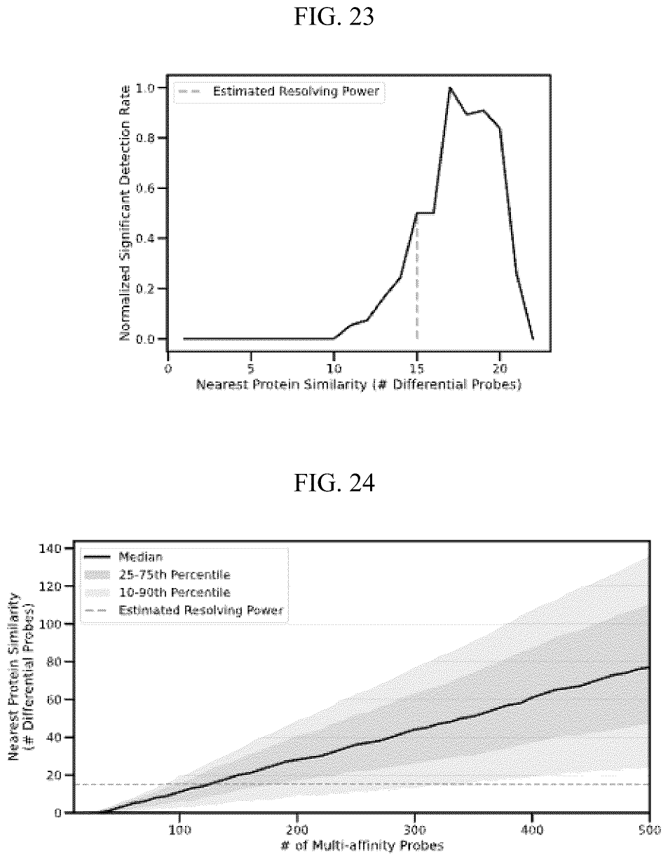

An observed binding pattern that resolves exclusively to a single protein in a database of candidate proteins can be identified as the target protein according to the decoding method set forth in this example. With 24 unique probes measured, model protein 1 (MP1) cannot yet be resolved from all proteins in the human proteome because certain proteins will be too similar to MP1 to resolve. Two similar proteins will have few “differential” probes predicted to bind to one protein (binding rate >0.2) but not the other. The MP1 binding data was searched against a full human proteome sequence database (Swiss-Prot; 20,261 sequences) with MP1 added. The resolving power of the decoding approach was estimated by iteratively removing proteins similar to MP1 from the database until MP1 detections were observed. A plot of the normalized significant detection rate vs. the nearest protein similarity is shown in FIG. 23. Significant detection rate was defined as detections in the MP1 sample minus detections in MP1 shuffled sample. The results indicated that MP1 was not detected unless all proteins in the database with 10 or fewer differential probes were filtered out (14,039 proteins pass filter). Detection further improved if only proteins with at least 17 differential probes were kept (261 proteins pass filter). Above this similarity threshold, the significant detection rate dropped due to an overly-constrained protein database yielding false detections. Based on these results, the resolving power of the decoding method was estimated at about 15 differential affinity reagents.

FIG. 24 shows how similar proteins in the human proteome are to their most-similar “nearest-neighbor” protein. The solid dark line shows the median similarity of a protein to its nearest-neighbor among the 20,261 proteins in the human proteome database. As more probes are measured, the number of differential probes between any two proteins increases. With 150 probes and 300 probes measured, 66% and 89% of human proteins are estimated to be detectable based on the current resolving power of the decoding method (nearest protein >15 differential probes).

While preferred embodiments of the present invention have been shown and described herein, it will be apparent to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of assaying proteins, comprising:
(a) contacting an array of different extant proteins with a plurality of different affinity reagents, wherein individual addresses of the array are each attached to an extant protein;
(b) acquiring empirical binding profiles from the individual addresses, the empirical binding profiles each comprising a plurality of binding outcomes for binding of an extant protein at one of the individual addresses to the plurality of different affinity reagents;
(c) providing a plurality of candidate outcome profiles, individual candidate outcome profiles of the plurality of candidate outcome profiles each comprising a plurality of statistical measures for a candidate protein, wherein the candidate proteins are known or suspected of being present in the sample;
(d) providing a plurality of pseudo outcome profiles, individual pseudo outcome profiles of the plurality of pseudo outcome profiles each including a plurality of statistical measures that is known to not occur for any of the candidate proteins;
(e) identifying extant proteins of the array based on the empirical binding profiles of the extant proteins and the plurality of candidate outcome profiles; and
(f) determining a false discovery statistic for the extant proteins based on the empirical binding profiles of the extant proteins and the plurality of pseudo outcome profiles.

2. The method of claim 1, wherein the pseudo outcome profiles are generated by modifying amino acid sequences of the candidate proteins and calculating statistical measures for the modified amino acid sequences.

3. The method of claim 1, wherein the plurality of pseudo outcome profiles comprises at least the same number of pseudo outcome profiles as the number of candidate outcome profiles in the plurality of candidate outcome profiles.

4. The method of claim 1, wherein the plurality of pseudo outcome profiles comprises a greater number of pseudo outcome profiles than the number of candidate outcome profiles in the plurality of candidate outcome profiles.

5. The method of claim 1, wherein the individual empirical binding profiles each comprise positive binding outcomes and negative binding outcomes.

6. The method of claim 5, wherein the individual candidate outcome profiles each comprise probabilities for positive binding outcomes and probabilities for negative binding outcomes, and wherein the individual pseudo outcome profiles each comprise probabilities for positive binding outcomes and probabilities for negative binding outcomes.

7. The method of claim 1, wherein the pseudo outcome profiles are generated from the candidate outcome profiles using a sequence agnostic approach.

8. The method of claim 7, wherein individual pseudo outcome profiles of the plurality of pseudo outcome profiles each comprise a rearrangement of a candidate outcome profile of the plurality of candidate outcome profiles.

9. The method of claim 8, wherein the individual empirical binding profiles each comprise a vector of empirical binding outcomes for the plurality of different affinity reagents with an individual extant protein.

10. The method of claim 9, wherein the individual candidate outcome profiles each comprise a vector of binding probabilities for the plurality of different affinity reagents with an individual candidate protein.

11. The method of claim 10, wherein the rearrangement comprises a shuffled order of the probabilities with respect to the different affinity reagents.

12. The method of claim 11, wherein the rearrangement comprises a reversed order of the probabilities with respect to the different affinity reagents.

13. The method of claim 1, wherein the identifying of step (e) comprises performing a process in a computer processor to identify extant proteins of the plurality of different extant proteins based on the probability of candidate outcome profiles being compatible with the empirical binding profiles of the extant proteins.

14. The method of claim 13, wherein step (e) further comprises outputting the identity of a given extant protein as the candidate protein having a candidate outcome profile with the most probable identity to the empirical binding profile of the given extant protein.

15. The method of claim 1, wherein the determining of step (f) comprises performing a process in a computer processor to determine a false discovery statistic based on the fraction of empirical binding profiles being more compatible with the pseudo outcome profiles than with the candidate outcome profiles.

16. The method of claim 15, wherein step (f) further comprises outputting a false identification rate for the identities of the plurality of different extant proteins.

17. The method of claim 15, wherein step (f) further comprises outputting a distribution of false identifications for the identities of the plurality of different extant proteins.

18. The method of claim 1, wherein the statistical measures comprise binary values.

19. The method of claim 1, wherein the statistical measures comprise analog values or non-binary values.

20. The method of claim 1, wherein the extant proteins are attached as single-molecules to the individual addresses, and wherein the binding data is acquired from the extant proteins at single-molecule resolution.

21. The method of claim 1, wherein the plurality of different affinity reagents comprises at least 100 different affinity reagents.

22. The method of claim 1, wherein the array comprises at least 1×10⁴ different extant proteins.

23. A method of assaying proteins, comprising:
(a) contacting an array of different extant proteins with a plurality of different affinity reagents, wherein individual addresses of the array are each attached to a single extant protein of the different extant proteins, and wherein the different affinity reagents recognize different extant proteins in the array;
(b) determining a binding outcome for each of the different affinity reagents at each of the individual addresses of the array;
(c) providing a database comprising a plurality of candidate proteins;
(d) providing a binding model for each of the different affinity reagents; and
(e) for an individual address in the array:
(i) adding a binding outcome of step (b) to a binding profile of the individual address;
(ii) evaluating the binding model to determine a collection of probabilities for each of the candidate proteins in the database producing the binding profile;
(iii) determining information entropy for the collection of probabilities; and
(iv) repeating steps (i) through (iii).

24. A detection system, comprising:
(a) a detector configured to detect binding outcomes for binding of a plurality of affinity reagents to an array of addresses, each of the addresses comprising an extant protein of a plurality of different extant proteins;
(b) a database comprising a plurality of candidate proteins;
(c) a binding model for each of the different affinity reagents; and
(d) a computer processor configured to:
(i) add a binding outcome of (a) to a binding profile of an individual address of the array;
(ii) evaluate the binding model to determine a collection of probabilities for each of the candidate proteins in the database producing the binding profile;
(iii) determine information entropy for the collection of probabilities; and
(iv) repeat (i) through (iii).


SEQUENCE LISTING (1 sequence)

SEQ ID NO: 1 - protein, 50 aa, synthetic construct
WNKKFRYFRRFRFWDDSDFHHHTGRTWTVPFHRHRETYRLTATDRGRWFH